SEQUENCE LISTING (2 sequences)

SEQ ID NO: 1 - DNA, 27 bp, Artificial Sequence
  Synthetic construct
catatggaccaggactcgctcagcagc

SEQ ID NO: 2 - DNA, 27 bp, Artificial Sequence
  Synthetic construct
ggatcctcaattctgcgtccggatggt

(19) United States
(12) Patent Application Publication
(10) Pub. No.: US 2012/0022024 A1
(43) Pub. Date: Jan. 26, 2012

(54) ANTI-BACTERIAL COMPOSITIONS AND METHODS INCLUDING TARGETING VIRULENCE FACTORS OF STAPHYLOCOCCUS AUREUS

(75) Inventors: Eric Oldfield, Champaign, IL (US); Yongcheng Song, Pearland, TX (US)

(73) Assignee: The Board of Trustees of the University of Illinois, Urbana, IL (US)

(21) Appl. No.: 13/188,218

(22) Filed: Jul. 21, 2011

Related U.S. Application Data
(63) Continuation of application No. PCT/US10/21800, filed on Jan. 22, 2010.
(60) Provisional application No. 61/146,864, filed on Jan. 23, 2009.

(51) Int. Cl. A61K 31/664; C07C 309/24; C07C 229/36; A61P 31/04; A61K 31/663; A61K 31/662; C07F 9/38; A61K 31/197; C07F 9/44; A61K 31/185

(52) U.S. Cl. 514/107; 562/15; 562/11; 562/44; 562/13; 562/444; 514/119; 514/117; 514/576; 514/120; 562/24; 514/563

(57) ABSTRACT

This disclosure relates to compositions and methods including for the inhibition, prevention, and/or treatment of microbial infections, including infections from such pathogens as Staphylococcus aureus.

### CROSS REFERENCE TO RELATED APPLICATIONS

This application is a continuation of International Application No. PCT/US2010/021800, filed Jan. 22, 2010, which claims the benefit of U.S. Provisional Patent Application No. 61/146,864 filed Jan. 23, 2009, both of which are hereby incorporated by reference in their entireties to the extent not inconsistent herewith.

### STATEMENT REGARDING FEDERALLY SPONSORED RESEARCH OR DEVELOPMENT

This invention was made, at least in part, with United States governmental support awarded by the National Institutes of Health grant numbers GM073216, GM65307, and AI074233. The United States Government has certain rights in this invention.

Each of the following applications and/or publications is also incorporated by reference in their entirety. U.S. Provisional Application Ser. No. 60/672,359 filed Apr. 18, 2005 entitled Novel Antimicrobial Therapy for Staphylococcus Aureus Infections by Nizet et al. and related International Application number PCT/US2006/014486 entitled Antimicrobial Therapy for Bacterial Infections filed Apr. 17, 2006 and published as International Application Publication number WO/2007/075186 on May 7, 2007. U.S. Provisional Application Ser. No. 60/800,654 filed May 12, 2006 entitled Antimicrobial Therapy for Bacterial Infections by Nizet et al. and related International Application number PCT/US2007/011466 filed May 11, 2007 entitled Antimicrobial Therapy for Bacterial Infections, published as International Application Publication number WO/2007/133712 on Nov. 22, 2007. U.S. patent application Ser. No. 11/918,584 filed Oct. 15, 2007 by Nizet et al. for Antimicrobial therapy for bacterial infections; published as US Patent Application Pub. No. 20090042976 on Feb. 12, 2009. U.S. patent application Ser. No. 12/299,934 filed Feb. 10, 2009 by Nizet et al. for Antimicrobial therapy for bacterial infections; published as US Patent Application Pub. No. 20090306021 on Dec. 10, 2009.

### BACKGROUND

Over the past 20 years, there has been an explosion in the prevalence of antibiotic resistant bacterial infections, both in the hospital and in the general community. In the United States, more deaths are now attributable to methicillin-resistant Staphylococcus aureus (MRSA) infections than to HIV/AIDS. Unfortunately, over the same time, there has been a decrease in the rate of discovery of new antibiotics, creating a pressing need for the development of novel infectious disease therapies. One approach involves the specific neutralization of bacterial virulence factors to render pathogenic bacteria susceptible to innate immune system clearance. However, conventional screening campaigns are not well suited for selecting such inhibitors, because virulence factors typically do not affect bacterial cell growth but rather exert their activity in vivo. In S. aureus, an important virulence factor is the carotenoid pigment staphyloxanthin. This pigment acts as an antioxidant, with its numerous conjugated double bonds enabling the detoxification of host immune system—generated reactive oxygen species (ROS) such as O₂⁻, H₂O₂, and HOCl. Bacteria that lack the carotenoid pigment grow normally, but they are rapidly killed by ROS from host neutrophils and are deficient in skin abscess formation. Blocking staphyloxanthin biosynthesis is therefore a potentially attractive therapeutic target, and the bright golden coloration of the virulence factor facilitates inhibitor screening.

Recently a novel approach to the prevention and treatment of drug resistant staphylococcal infections was developed, targeting an important virulence factor of the bacterium, called staphyloxanthin (STX), which is used by S. aureus to resist the human immune system (neutrophils). See International Application Publication number WO/2007/133712. What was found is that phosphonosulfonates, a class of compounds (human squalene synthase (SQS) inhibitors) previously advanced to clinical trials to lower cholesterol level in humans, are able to inhibit staphyloxanthin biosynthesis in S. aureus. These compounds were found to be potent inhibitors of dehydrosqualene synthase (CrtM), the first enzyme committed in the staphyloxanthin biosynthesis. In an animal model of systemic staphylococcal infection, the compound, BPH-652 (FX24B-04-652), was found to inhibit the bacterial growth by 98%. Embodiments of the present invention including compounds and methods are useful to meet significant needs in connection with anti-infective technology.

### SUMMARY OF THE INVENTION

Embodiments of the invention generally relate to the treatment of infectious agents by disruption of certain biosynthetic or biochemical pathways. In an aspect, novel compounds of the present invention may be used to selectively inhibit one or more biosynthetic or biochemical pathways of an infectious agent over one or more biosynthetic or biochemical pathways of a host. In another aspect, novel compounds of the invention include phosphonoacetohydroxamates and phosphonoacetamides which, as a class, have been found to disrupt biochemical and biosynthetic pathways of infectious agents, including Staphylococcus aureus. In a further aspect, novel compounds of the present invention may be used with methods of the present invention to selectively inhibit biosynthetic or biochemical pathways of an infectious agent over biosynthetic or biochemical pathways of a host which may be infected by such agent.

In embodiments, the invention provides novel compounds of the formula

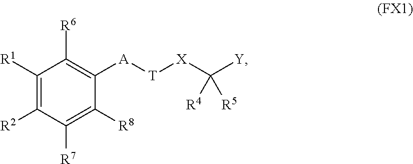

wherein:
n is 0, 1, 2, 3, 4, 5, 6, 7, 8, 9, or 10;
Y is selected from the group consisting of: —P(O)(O⁻M¹)(O⁻M²), —P(O)(OH)₂, —P(O)(OH)(O⁻M³), —SO₃H, —SO₃⁻M⁴, —C(O)O⁻M⁵ and —COOH;
X is selected from the group consisting of: —C(O)—, —S(O₂)—, —P(O)(O⁻M⁶)-, and —CH₂—;
T is selected from the group consisting of: —O—, —CH₂—, and —NR³—;
A is a bridging diradical selected from the group consisting of: —(CH₂)ₙ—, —(C₆H₄)ₙ—, —(CF₂)ₙ—, —(CCl₂)ₙ—, —(CBr₂)ₙ—, alkylene, substituted alkylene, arylene, substituted arylene, alkylenearylene, substituted alkylenearylene, arylenealkylene, substituted arylenealkylene, alkylenearylenealkylene, substituted alkylenearylenealkylene, oxyalkylene, substituted oxyalkylene, oxyalkylenearylene, substituted oxyalkylenearylene, oxyarylene, substituted oxyarylene, oxyarylenealkylene, and substituted oxyarylenealkylene.
M¹, M², M³, M⁴, M⁵, M⁶, M⁷ are each independently a pharmaceutically acceptable cation;
R³ is selected from the group consisting of: —H, —OH, —O⁻M⁷, aryl, substituted aryl, alkyl, substituted alkyl, —COOH, —COO⁻, —CO—NH₂, —(CH₂)ₙ—O—CO—, and halo;
R⁴ and R⁵ are each independently selected from the group consisting of —H, —OH, —O⁻M⁷, aryl, substituted aryl, alkyl, substituted alkyl, —COOH, —COO⁻, —CO—NH₂, —(CH₂)ₙ—COOH, —(CH₂)ₙ—COO⁻, and halo;
R¹, R², R³, R⁶, R⁷, R⁸ are each independently selected from the group consisting of: aryl, substituted aryl, alkyl, substituted alkyl, —COOH, —COO⁻, —CO—NH₂, —(CH₂)ₙ—O—CO—, halo, and the following substituents:

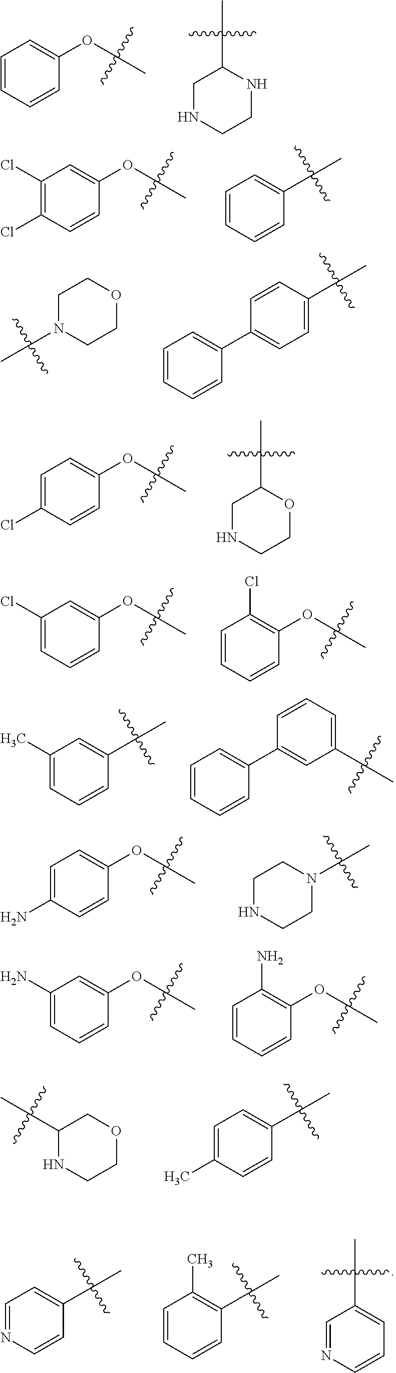

In further embodiments, the invention provides the following specific compounds:

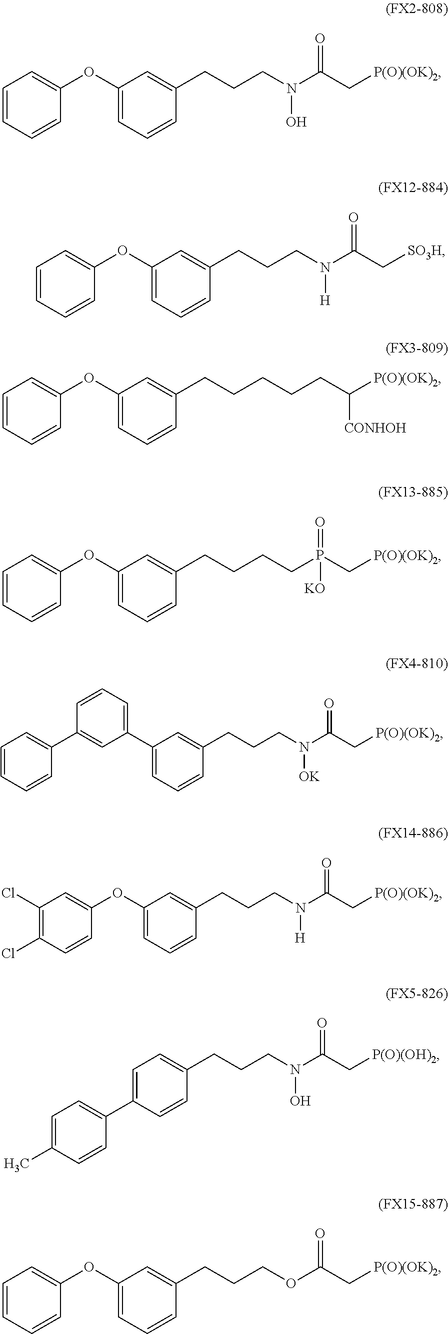

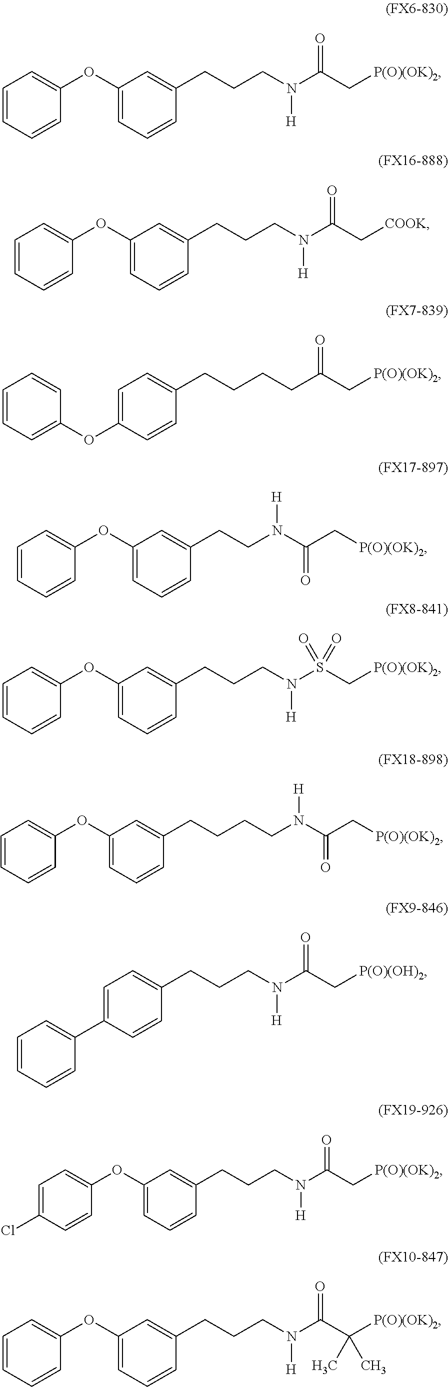

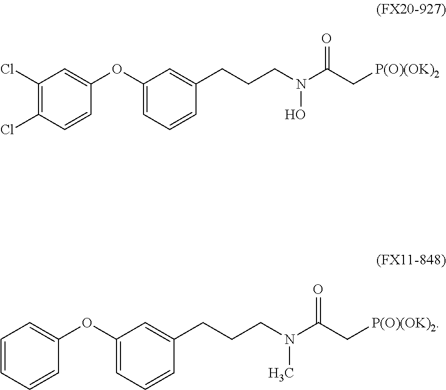

In further embodiments, the invention provides a method of inhibiting an infection comprising contacting an infectious agent with a compound of the invention. In an embodiment of this method of the present invention the infection is a microbial infection. In a further embodiment the infectious agent is a Staphylococcus species including Staphylococcus aureus. In another embodiment the compounds of the present invention are capable of inhibiting dehydrosqualene synthase (CrtM) or production of staphyloxanthin (STX).

In a further alternative embodiment of the present invention, the invention provides a method of inhibiting an infection comprising contacting an infectious agent with a compound of the invention in combination with at least one antibiotic. In an aspect of this embodiment the antibiotic is or belongs to a class selected from the group consisting of aminoglycosides, penicillins, cephalosporins, carbapenems, monobactams, quinolones, tetracyclines, glycopeptides, chloramphenicol, clindamycin, trimethoprim, sulfamethoxazole, nitrofurantoin, rifampin and mupirocin. In another aspect of this embodiment the antibiotic is selected from the group consisting of amikacin, gentamicin, kanamycin, netilmicin, tobramycin, streptomycin, azithromycin, clarithromycin, erythromycin, erythromycin estolate, erythromycin ethylsuccinate, erythromycin gluceptatellactobionate, erythromycin stearate, penicillin G, penicillin V, methicillin, nafcillin, oxacillin, cloxacillin, dicloxacillin, ampicillin, amoxicillin, ticarcillin, carbenicillin, mezlocillin, azlocillin, piperacillin, cephalothin, cefazolin, cefaclor, cefamandole, cefoxitin, cefuroxime, cefonicid, cefmetazole, cefotetan, cefprozil, loracarbef, cefetamet, cefoperazone, cefotaxime, ceftizoxime, ceftriaxone, ceftazidime, cefepime, cefixime, cefpodoxime, cefsulodin, imipenem, aztreonam, fleroxacin, nalidixic acid, norfloxacin, ciprofloxacin, ofloxacin. enoxacin, lomefloxacin, cinoxacin, doxycycline, minocycline, tetracycline, vancomycin, and teicoplanin.

In an alternative embodiment a method of inhibiting growth of a microbe is provided comprising contacting the microbe with a compound of the invention. In an aspect of this embodiment the microbe is a Staphylococcus species. In an alternative embodiment of this aspect a method of selectively inhibiting microbial activity is provided comprising contacting a microbe with a compound of the invention wherein the compound is capable of inhibiting CrtM activity or STX biosynthesis and has a limited capability for inhibiting or substantially inhibiting human cholesterol biosynthesis or human squalene synthase (hSQS).

In a further alternative embodiment a method of inhibiting growth of a microbe is provided comprising contacting the microbe with a compound of the invention in combination with at least one antibiotic. In an aspect of this embodiment the antibiotic is or belongs to a class selected from the group consisting of aminoglycosides, penicillins, cephalosporins, carbapenems, monobactams, quinolones, tetracyclines, glycopeptides, chloramphenicol, clindamycin, trimethoprim, sulfamethoxazole, nitrofurantoin, rifampin and mupirocin. In another aspect of this embodiment the antibiotic is selected from the group consisting of amikacin, gentamicin, kanamycin, netilmicin, tobramycin, streptomycin, azithromycin, clarithromycin, erythromycin, erythromycin estolate, erythromycin ethylsuccinate, erythromycin gluceptatellactobionate, erythromycin stearate, penicillin G, penicillin V, methicillin, nafcillin, oxacillin, cloxacillin, dicloxacillin, ampicillin, amoxicillin, ticarcillin, carbenicillin, mezlocillin, azlocillin, piperacillin, cephalothin, cefazolin, cefaclor, cefamandole, cefoxitin, cefuroxime, cefonicid, cefmetazole, cefotetan, cefprozil, loracarbef, cefetamet, cefoperazone, cefotaxime, ceftizoxime, ceftriaxone, ceftazidime, cefepime, cefixime, cefpodoxime, cefsulodin, imipenem, aztreonam, fleroxacin, nalidixic acid, norfloxacin, ciprofloxacin, ofloxacin. enoxacin, lomefloxacin, cinoxacin, doxycycline, minocycline, tetracycline, vancomycin, and teicoplanin.

In a further alternative embodiment a method is provided of contacting a microbe with a compound of the present invention which is capable of inhibiting STX biosynthesis with an IC₅₀ level of less than or equal to 50 μM or is capable of inhibiting CrtM activity with an IC₅₀ level of less than or equal to 500 μM. In a further aspect of this embodiment a compound of the present invention has an IC₅₀ level for STX of less than or equal to 10 μM, less than or equal to 1 μM, less than or equal to 100 nM or less than or equal to 50 nM. In an alternative further aspect of this embodiment a compound of the present invention has an IC₅₀ level for CrtM less than or equal to 100 μM.

In a further alternative embodiment a method is provided of contacting a microbe with a compound of the present invention which is capable of inhibiting STX biosynthesis in combination with at least one antibiotic. In an aspect of this embodiment the antibiotic is or belongs to a class selected from the group consisting of aminoglycosides, penicillins, cephalosporins, carbapenems, monobactams, quinolones, tetracyclines, glycopeptides, chloramphenicol, clindamycin, trimethoprim, sulfamethoxazole, nitrofurantoin, rifampin and mupirocin. In another aspect of this embodiment the antibiotic is selected from the group consisting of amikacin, gentamicin, kanamycin, netilmicin, tobramycin, streptomycin, azithromycin, clarithromycin, erythromycin, erythromycin estolate, erythromycin ethylsuccinate, erythromycin gluceptatellactobionate, erythromycin stearate, penicillin G, penicillin V, methicillin, nafcillin, oxacillin, cloxacillin, dicloxacillin, ampicillin, amoxicillin, ticarcillin, carbenicillin, mezlocillin, azlocillin, piperacillin, cephalothin, cefazolin, cefaclor, cefamandole, cefoxitin, cefuroxime, cefonicid, cefmetazole, cefotetan, cefprozil, loracarbef, cefetamet, cefoperazone, cefotaxime, ceftizoxime, ceftriaxone, ceftazidime, cefepime, cefixime, cefpodoxime, cefsulodin, imipenem, aztreonam, fleroxacin, nalidixic acid, norfloxacin, ciprofloxacin, ofloxacin. enoxacin, lomefloxacin, cinoxacin, doxycycline, minocycline, tetracycline, vancomycin, and teicoplanin.

In a further embodiment of the present invention, the limited capability of a compound of the present invention for inhibiting or substantially inhibiting human cholesterol biosynthesis or human squalene synthase is capable of being reflected by a relative selectivity of the compound for inhibiting CrtM activity or inhibiting STX biosynthesis in comparison to inhibiting human squalene synthase (hSQS), wherein the compound is capable of demonstrating said relative selectivity in the form of a selectivity ratio of [IC₅₀(hSQS)/IC₅₀(CrtM)] for the compound with respect to that of a reference compound BPH-652 (FX24B-04-652), and wherein said relative selectivity value is greater than 1, 10, 100, or 200; or wherein said limited capability is reflected by the compound being capable of demonstrating an absolute ratio of [IC₅₀(hSQS)/IC₅₀(CrtM)] wherein such absolute ratio is greater than 0.005, 0.05, 0.2, or 0.5.

In alternative embodiments the invention provides compounds of the formula FX21-I or FX22-II which may be used in any method of the present invention:

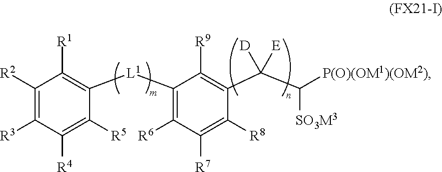

wherein:
m is 0, 1, 2 or 3;
n is 1, 2, 3, 4, 5, 6, 7, 8, 9, or 10;
D and E are each independently selected from the group consisting of: H, aryl, substituted aryl, alkyl, substituted alkyl, carboxyl, aminocarbonyl, alkylsulfonylaminocarboxyl, alkoxycarbonyl, and halo;
M¹, M², and M³ are each independently a pharmaceutically acceptable cation;
R¹ is selected from the group consisting of: H, aryl, substituted aryl, alkyl, substituted alkyl, carboxyl, aminocarbonyl, alkylsulfonylaminocarboxyl, alkoxycarbonyl, and halo, or R¹ and R², together with the carbons to which they are bound, can be joined to form a 4 to 7 membered ring or a substituted 4 to 7 membered ring;
R² is selected from the group consisting of: H, aryl, substituted aryl, alkyl, substituted alkyl, carboxyl, aminocarbonyl, alkylsulfonylaminocarboxyl, alkoxycarbonyl, and halo, or R² and R¹, together with the carbons to which they are bound, can be joined to form a 4 to 7 membered ring or a substituted 4 to 7 membered ring, or R² and R³, together with the carbons to which they are bound, can be joined to form a 4 to 7 membered ring or a substituted 4 to 7 membered ring;
R³ is selected from the group consisting of: H, aryl, substituted aryl, alkyl, substituted alkyl, carboxyl, aminocarbonyl, alkylsulfonylaminocarboxyl, alkoxycarbonyl, and halo, or R³ and R², together with the carbons to which they are bound, can be joined to form a 4 to 7 membered ring or a substituted 4 to 7 membered ring, or R³ and R⁴, together with the carbons to which they are bound, can be joined to form a 4 to 7 membered ring or a substituted 4 to 7 membered ring;
R⁴ is selected from the group consisting of: H, aryl, substituted aryl, alkyl, substituted alkyl, carboxyl, aminocarbonyl, alkylsulfonylaminocarboxyl, alkoxycarbonyl, and halo, or R⁴ and R³, together with the carbons to which they are bound, can be joined to form a 4 to 7 membered ring or a substituted 4 to 7 membered ring;
R⁵, R⁶, R⁷, R⁸, and R⁹ are each independently selected from the group consisting of: H, aryl, substituted aryl, alkyl, substituted alkyl, carboxyl, aminocarbonyl, alkylsulfonylaminocarboxyl, alkoxycarbonyl, and halo;
L¹ is —S—, —SO—, —SO₂—, —O—, —N(R¹⁹)—, or —C(R²⁰)(R²¹)—; wherein R¹⁹, R²⁰ and R²¹ are each independently selected from the group consisting of: H, aryl, substituted aryl, alkyl, substituted alkyl, carboxyl, aminocarbonyl, alkylsulfonylaminocarboxyl, alkoxycarbonyl, and halo;

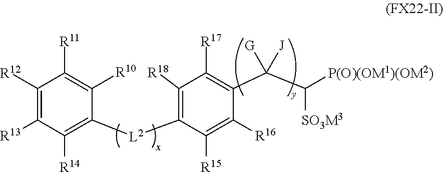

wherein:
x is 0, 1, 2, or 3;
y is 1, 2, 3, 4, 5, 6, 7, 8, 9, or 10;
G and J are independently selected from the group consisting of: H, aryl, substituted aryl, alkyl, substituted alkyl, carboxyl, aminocarbonyl, alkylsulfonylaminocarboxyl, alkoxycarbonyl, and halo;
M⁴, M⁵, and M⁶ are each independently a pharmaceutically acceptable cation;
R¹⁰ is selected from the group consisting of: H, aryl, substituted aryl, alkyl, substituted alkyl, carboxyl, aminocarbonyl, alkylsulfonylaminocarboxyl, alkoxycarbonyl, and halo, or R¹⁰ and R¹¹, together with the carbons to which they are bound, can be joined to form a 4 to 7 membered ring or a substituted 4 to 7 membered ring;
R¹¹ is selected from the group consisting of: H, aryl, substituted aryl, alkyl, substituted alkyl, carboxyl, aminocarbonyl, alkylsulfonylaminocarboxyl, alkoxycarbonyl, and halo, or R¹¹ and R¹⁰, together with the carbons to which they are bound, can be joined to form a 4 to 7 membered ring or a substituted 4 to 7 membered ring, or R¹¹ and R¹², together with the carbons to which they are bound, can be joined to form a 4 to 7 membered ring or a substituted 4 to 7 membered ring;
R¹² is selected from the group consisting of: H, aryl, substituted aryl, alkyl, substituted alkyl, carboxyl, aminocarbonyl, alkylsulfonylaminocarboxyl, alkoxycarbonyl, and halo, or R¹² and R¹¹, together with the carbons to which they are bound, can be joined to form a 4 to 7 membered ring or a substituted 4 to 7 membered ring, or R¹² and R¹³, together with the carbons to which they are bound, can be joined to form a 4 to 7 membered ring or a substituted 4 to 7 membered ring;
R¹³ is selected from the group consisting of: H, aryl, substituted aryl, alkyl, substituted alkyl, carboxyl, aminocarbonyl, alkylsulfonylaminocarboxyl, alkoxycarbonyl, and halo, or R¹³ and R¹², together with the carbons to which they are bound, can be joined to form a 4 to 7 membered ring or a substituted 4 to 7 membered ring;
R¹⁴, R¹⁵, R¹⁶, R¹⁷, and R¹⁸ are each independently selected from the group consisting of: H, aryl, substituted aryl, alkyl, substituted alkyl, carboxyl, aminocarbonyl, alkylsulfonylaminocarboxyl, alkoxycarbonyl, and halo;
L² is —S—, —SO—, —SO₂—, —O—, —N(R²²)—, or —C(R²³)(R²⁴)—; wherein R²², R²³ and R²⁴ are each independently selected from the group consisting of: H, aryl, substituted aryl, alkyl, substituted alkyl, carboxyl, aminocarbonyl, alkylsulfonylaminocarboxyl, alkoxycarbonyl, and halo.

In further embodiments, the invention provides compounds of the general formulae FX21-I and FX22-II which may be used in any method of the present invention and are not of the specific formulae:

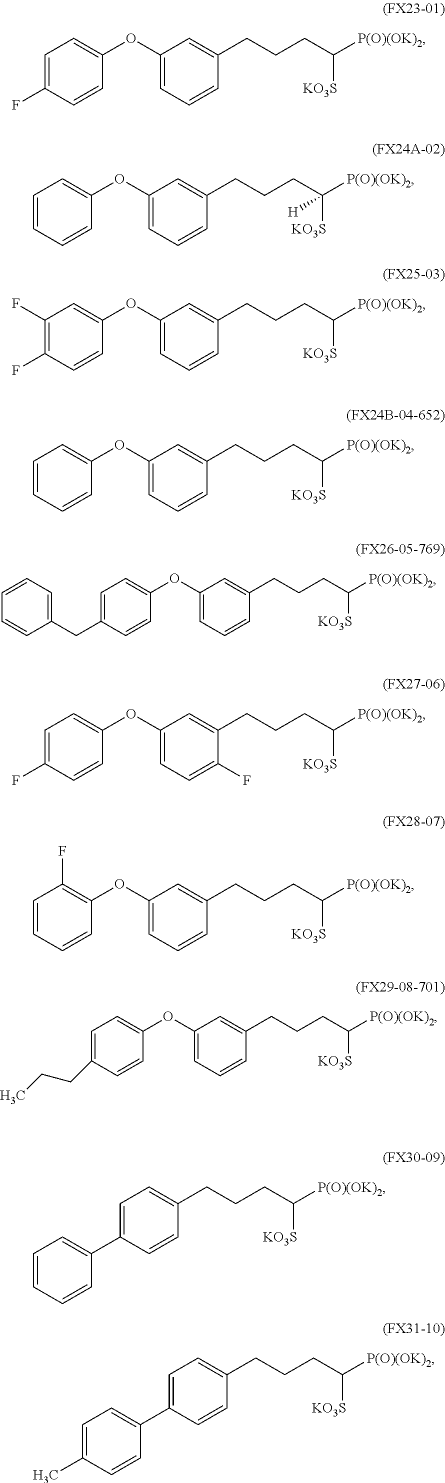

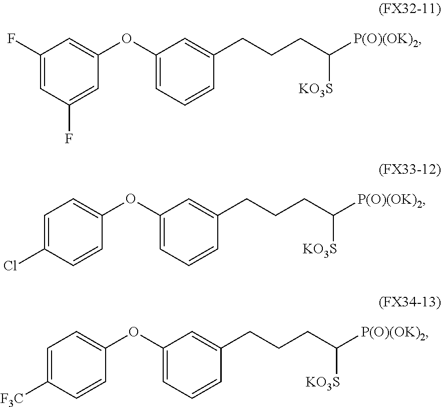

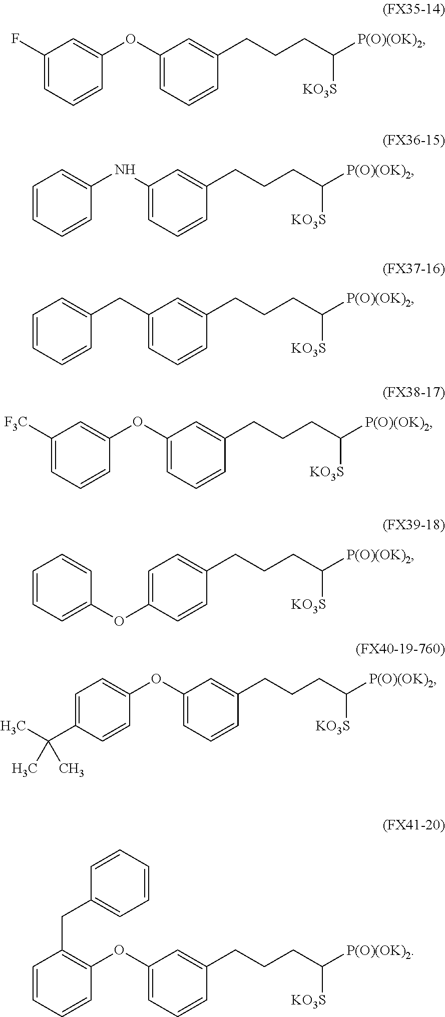

In an embodiment, the invention provides a method of preventing or treating a microbial infection comprising administering to a subject in need thereof a compound of the invention.

In a further alternative embodiment, the invention provides a method of preventing or treating a microbial infection comprising administering to a subject in need thereof a compound of the invention in combination with at least one antibiotic. In an aspect of this embodiment the antibiotic is or belongs to a class selected from the group consisting of aminoglycosides, penicillins, cephalosporins, carbapenems, monobactams, quinolones, tetracyclines, glycopeptides, chloramphenicol, clindamycin, trimethoprim, sulfamethoxazole, nitrofurantoin, rifampin and mupirocin. In another aspect of this embodiment the antibiotic is selected from the group consisting of amikacin, gentamicin, kanamycin, netilmicin, tobramycin, streptomycin, azithromycin, clarithromycin, erythromycin, erythromycin estolate, erythromycin ethylsuccinate, erythromycin gluceptatellactobionate, erythromycin stearate, penicillin G, penicillin V, methicillin, nafcillin, oxacillin, cloxacillin, dicloxacillin, ampicillin, amoxicillin, ticarcillin, carbenicillin, mezlocillin, azlocillin, piperacillin, cephalothin, cefazolin, cefaclor, cefamandole, cefoxitin, cefuroxime, cefonicid, cefmetazole, cefotetan, cefprozil, loracarbef, cefetamet, cefoperazone, cefotaxime, ceftizoxime, ceftriaxone, ceftazidime, cefepime, cefixime, cefpodoxime, cefsulodin, imipenem, aztreonam, fleroxacin, nalidixic acid, norfloxacin, ciprofloxacin, ofloxacin. enoxacin, lomefloxacin, cinoxacin, doxycycline, minocycline, tetracycline, vancomycin, and teicoplanin.

In an embodiment, the invention provides the use of a compound in the manufacture of a medicament. In an embodiment, the invention provides the use of a compound for the prevention or treatment of an infection. In an embodiment, the invention provides the use of a compound in the manufacture of a medicament for the prevention or treatment of an infection. In an embodiment, the invention provides the use of a medicament. In an embodiment, the invention provides the use of a compound in the manufacture of a disinfectant.

In an embodiment, the invention provides a method of disinfecting a surface, substance or object comprising administering to the surface, substance or object a compound of the invention.

### BRIEF DESCRIPTION OF THE DRAWINGS

FIG. 1 shows several biosynthetic pathways which are relevant to aspects of the present invention. FIG. 1 shows staphyloxanthin biosynthesis in S. aureus. FIG. 1 shows cholesterol biosynthesis in humans and ergosterol biosynthesis in, e.g., yeasts and some parasitic protozoa.

FIG. 2 shows representative dose-response curves of the staphyloxanthin inhibition in S. aureus for selected phosphonosulfonate compounds.

FIG. 3 shows representative dose-response curves of pigment inhibition in S. aureus for selected phosphonosulfonate compounds.

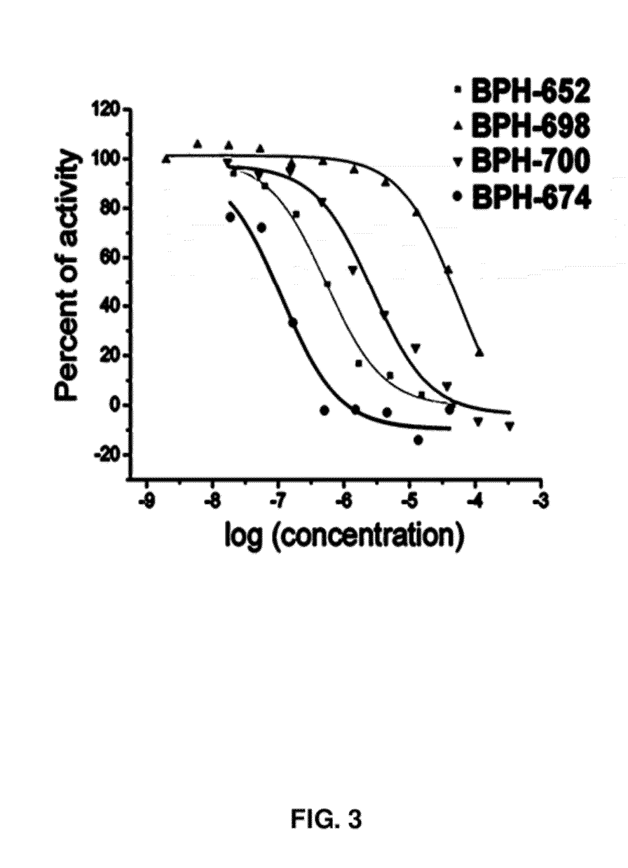

### DETAILED DESCRIPTION OF THE INVENTION

As used herein, the term “BPH-652” refers to the phosphonosulfonate compound having the structure

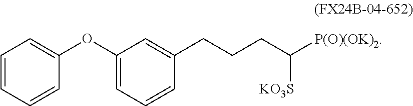

As used herein, the term “Infection” refers to the detrimental colonization of a host organism by a foreign species. The foreign species is also referred to herein as an “infectious agent.” Examples of infectious agents include, but are not limited to, bacteria such as Mycobacterium tuberculosis and Pseudomonas, and viruses such as Adenoviridae and Picornaviridae.

As used herein, the term “Microbe” refers to an organism that is too small to be seen by the naked eye. Examples of microbes include, but are not limited to, bacteria, fungi, archaea, protists, viruses, prions, some plankton, planarian and amoeba. The detrimental colonization of a host organism by a microbe is also referred to herein as a “Microbial Infection.”

As used herein, the term “IC₅₀ Level”, as would be understood in the art, generally refers to a measure of the effectiveness of a compound in inhibiting biological or biochemical function and is a quantitative measure which indicates how much of a particular drug or other substance is needed to inhibit a given activity or process (or component of a process, i.e. an enzyme, cell, cell receptor or microorganism) at half of a reference level. The related term “IC₅₀(Compound)” as used herein refers to the IC₅₀ level of the specific compound indicated.

As used herein, the terms “Selectivity Ratio” and “Absolute Selectivity Ratio” refer to a ratio of the IC₅₀ inhibition level of a compound for a biological or biochemical function to the IC₅₀ inhibition level of the same compound for a different biological or biochemical function.

As used herein, the term “Relative Selectivity Ratio” refers to a Selectivity Ratio which is normalized to the Selectivity Ratio of a reference compound for the same biological or biochemical functions. For example, the selectivity ratio of a compound for the inhibition of CrtM with respect to hSQS may be normalized to the selectivity ratio of BPH-652 (FX24B-04-652) for the inhibition of CrtM with respect to hSQS.

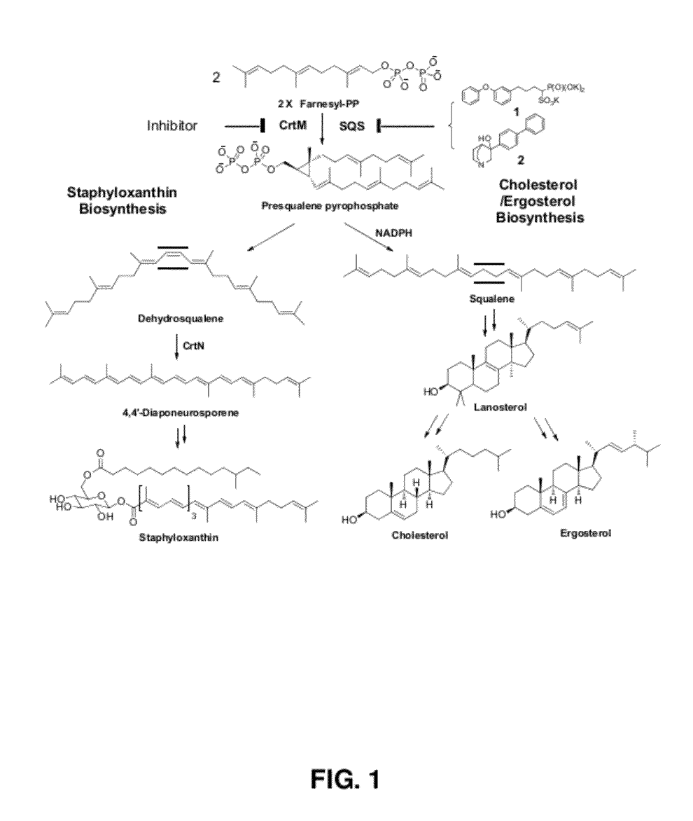

In an aspect of the invention, a method is provided for the selective inhibition of a biochemical or biosynthetic pathway in S. aureus over the inhibition of a biochemical or biosynthetic pathway in a human host. FIG. 1 shows several biosynthetic pathways which may be affected by exposure to compounds described herein. As can be seen in FIG. 1, each biosynthetic pathway involves initial formation of presqualene diphosphate, catalyzed by CrtM (S. aureus) or by squalene synthase (SQS). In S. aureus, the NADPH reduction step is absent, resulting in production of dehydrosqualene, not squalene.

As used herein, the term “alkyl” refers to a monoradical of a branched or unbranched (straight-chain or linear) saturated hydrocarbon and to cycloalkyl groups having one or more rings. Unless otherwise indicated alkyl groups have 1 to 30 carbon atoms, preferred alkyls have 1-22 carbon atoms. Shorter alkyl groups are those having 1 to 6 carbon atoms including methyl, ethyl, propyl, butyl, pentyl and hexyl groups, including all isomers thereof. Longer alkyl groups are those having 8-22 carbon atoms and preferably those having 12-22 carbon atoms, as well as those having 12-20 and those having 16-18 carbon atoms. The term “cycloalkyl” refers to cyclic alkyl groups having preferably 3 to 30 carbon atoms (preferably having 1-22 carbon atoms) having a single cyclic ring or multiple condensed rings. Cycloalkyl groups include among others those having 5, 6, 7, 8, 9 or 10 carbon ring members. Cycloalkyl groups include, by way of example, single ring structures such as cyclopropyl, cyclobutyl, cyclopentyl, cyclohexyl, cyclooctyl, and the like, or multiple ring structures such as adamantanyl, and the like. Unless otherwise indicated alkyl groups including cycloalkyl groups are optionally substituted as defined below. The term alkoxy (or alkoxide) refers to a —O-alkyl group, where alkyl groups are as defined above.

The term “aryl” refers to a monoradical containing at least one aromatic ring. The radical is formally derived by removing a hydrogen from an aromatic ring carbon. Aryl groups contain one or more rings at least one of which is aromatic. Rings of aryl groups may be linked by a single bond or a linker group or may be fused. Exemplary aryl groups include phenyl, biphenyl and naphthyl groups. Aryl groups include those having from 6 to 30 carbon atoms and those containing 6-12 carbon atoms. Unless otherwise noted aryl groups are optionally substituted as described herein.

The term “amino” refers generically to a —N(R″)₂ group wherein each R″, independently, is hydrogen, alkyl, alkenyl, alkynyl, aryl, heterocyclic, or heteroaryl radical as described above. Two of R″ may be linked to form a heterocyclic ring containing at least one nitrogen. An “alkyl amino” group refers to an amino group wherein at least one R″ is alkyl. An “aryl amino” group refers to an amino group wherein at least one R″ is aryl. Amino groups may contain aryl and alkyl groups.

The term “amido” refers generically to an —CO—N(R″)₂ group wherein R″ independently of other R″ is hydrogen, alkyl, alkenyl, alkynyl, aryl, heterocyclic, or heteroaryl radical as described above. Two of R″ may be linked to form a ring. An “alkyl amido” group refers to an amido group wherein at least one R″ is alkyl. An “aryl amido” group refers to an amido group wherein at least one R″ is aryl. Amido groups may contain aryl and alkyl groups.

The term “aminoacyl” group” refers generically to an —NR′—CO—R′ group wherein R′ independently of other R′ is hydrogen, alkyl, alkenyl, alkynyl, aryl, heterocyclyl, or heteroaryl radical as described above. Two of R′ may be linked to form a ring. An “alkyl aminoacyl” group refers to an aminoacyl group wherein at least one R′ is alkyl. An “aryl amido” group refers to an aminoacyl group wherein at least one R′ is aryl.

The term “alkylene” refers to a diradical of a branched or unbranched saturated hydrocarbon chain, which unless otherwise indicated can have 1 to 12 carbon atoms, or 1-6 carbon atoms, or 2-4 carbon atoms. This term is exemplified by groups such as methylene (—CH₂—), ethylene (—CH₂CH₂—), more generally —(CH₂)ₙ— where n is 1-12 or preferably 1-6 or n is 1, 2, 3 or 4. —(CH₂)ₙ—, where n is 0 indicates the absence of the indicated linker. Alkylene groups may be branched, e.g., by substitution with alkyl group substituents. Alkylene groups may be optionally substituted as described herein. Alkylene groups may have up to two non-hydrogen substituents per carbon atoms. Preferred substituted alkylene groups have 1, 2, 3 or 4 non-hydrogen substituents.

Alkyl and aryl groups may be substituted or unsubstituted. These groups may contain non-hydrogen substituents dependent upon the number of carbon atoms in the group and the degree of unsaturation of the group. Unless otherwise indicated substituted alkyl and aryl groups preferably contain 1-10, and more preferably 1-6, and more preferably 1, 2 or 3 non-hydrogen substituents.

Optional substitution refers to substitution with one or more of the following functional groups: Halogens (e.g., Br—, I—, Cl—, F—), nitro groups (NO₂—), cyano (NC—), isocyano (CN—), thiocyano (NCS—), isothiocyano (SCN—), sulfuryl (SO₂—), —N(R′)₂, —OR′, or —SR′ (where each R′, independently, is hydrogen, alkyl, alkenyl, alkynyl, or aryl), alkyl, alkenyl, alkynyl, aryl, heteroaryl, heterocyclic groups or optional substituents of groups described herein.

As to any of the above groups or linkers which contain one or more substituents, it is understood, that such groups or linkers do not contain any substitution or substitution patterns which are sterically impractical and/or synthetically non-feasible. In addition, the compounds of this invention include all stereochemical isomers arising from the substitution of these compounds.

Methods for synthesizing compounds described herein are well known in the art or may be identified by routine modification of art-known synthetic methods, such as those provided in Example 3, below. General methods for synthesis of organic compounds may be found in the following references: Advanced Organic Chemistry—Part B: Reactions and Synthesis (5th Edition), Carey, Francis A. and Sundberg, Richard J., Springer-Verlag; The Organic Chemistry of Drug Synthesis, Daniel Lednicer and Lester A. Mitscher, Wiley-Interscience (2008); and March's Advanced Organic Chemistry Reactions, Mechanisms, and Structure (6th Edition), Michael B. Smith and Jerry March, Wiley-Interscience.

The compounds of the present inventions form salts which are also within the scope of this invention. Reference to a compound formula herein is understood to include reference to salts thereof, unless otherwise indicated. The term “salt(s)”, as employed herein, denotes acidic and/or basic salts formed with inorganic and/or organic acids and bases. In addition, when a compound contains both a basic moiety, such as, but not limited to an amine or a pyridine ring, and an acidic moiety, such as, but not limited to, a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful, e.g., in isolation or purification steps which may be employed during preparation. Salts of the compounds disclosed herein may be formed, for example, by reacting a compound with an amount of acid or base, such as an equivalent amount, in a medium such as one in which the salt precipitates or in an aqueous medium followed by lyophilization. One of ordinary skill in the art and familiar with nuances of nomenclature will appreciate that the teachings herein, including as described and particularly exemplified, extend to include derivatives of compounds herein such as esters.

Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

Exemplary basic salts include salts formed from cations, such as ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines [formed with N,N-bis(dehydro-abietyl)ethylenediamine], N-methyl-D-glucamines, N-methyl-D-glucamides, t-butyl amines, and salts with amino acids such as arginine, lysine and the like. Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others. Pharmaceutically acceptable (i.e., non-toxic, physiologically acceptable) cations and salts thereof are preferred, although other salts are also useful, e.g., in isolation or purification steps which may be employed during preparation.

Compounds of the present invention, and salts thereof, may exist in their tautomeric form, in which hydrogen atoms are transposed to other parts of the molecules and the chemical bonds between the atoms of the molecules are consequently rearranged. It should be understood that all tautomeric forms, insofar as they may exist, are included within the invention. Additionally, inventive compounds may have trans and cis isomers and may contain one or more chiral centers, and therefore exist in enantiomeric and diastereomeric forms. The invention includes all such isomers, as well as mixtures of cis and trans isomers, mixtures of diastereomers and racemic mixtures of enantiomers (optical isomers). When no specific mention is made of the configuration (cis, trans or R or S) of a compound (or of an asymmetric carbon), then any one of the isomers or a mixture of more than one isomer is intended. The processes for preparation can use racemates, enantiomers, or diastereomers as starting materials. When enantiomeric or diastereomeric products are prepared, they can be separated by conventional methods, for example, by chromatographic or fractional crystallization. The inventive compounds may be in the free or hydrate form.

Compounds of the invention may have prodrug forms. Prodrugs of the compounds of the invention are useful in the methods of this invention. Any compound that will be converted in vivo to provide a biologically, pharmaceutically or therapeutically active form of a compound of the invention is a prodrug. Various examples and forms of prodrugs are well known in the art. Examples of prodrugs may be found in Design of Prodrugs, edited by H. Bundgaard, (Elsevier, 1985), Methods in Enzymology, Vol. 42, at pp. 309-396, edited by K. Widder, et. al. (Academic Press, 1985); A Textbook of Drug Design and Development, edited by Krosgaard-Larsen and H. Bundgaard, Chapter 5, “Design and Application of Prodrugs,” by H. Bundgaard, at pp. 113-191, 1991); H. Bundgaard, Advanced Drug Delivery Reviews, Vol. 8, p. 1-38 (1992); H. Bundgaard, et al., Journal of Pharmaceutical Sciences, Vol. 77, p. 285 (1988); and Nogrady (1985) Medicinal Chemistry A Biochemical Approach, Oxford University Press, New York, pages 388-392.

### Example 1

### Phosphonosulfonates as Selective Inhibitors of Dehydrosqualene Synthase and Staphyloxanthin Biosynthesis in Staphylococcus aureus

In this Example, we disclose potent and specific CrtM inhibitors, all of which are phosphonosulfonates. The rationale which we determined for finding selective inhibitors is that since BPH-652 (FX24B-04-652) is also a very potent human squalene synthase inhibitor, it causes the formation of the 1,10-dioic acid FPP metabolite. This is undesirable, so specific CrtM inhibitors (without SQS activity) are needed. We now have made a series of phosphonosulfonate compounds and tested their activities on CrtM and human SQS, as well as in staphyloxanthin (STX) biosynthesis inhibition in S. aureus (cell activity). We have found that several phosphonosulfonate compounds, shown in the table below, are potent, selective inhibitors of S. aureus CrtM and in S. aureus cells, with no or very weak off-target (human squalene synthase) activity. As such, they will not interfere with human sterol/steroid biosynthesis. These compounds and results are given in Table 1, below.

TABLE 1
Phophonosulfonate Compounds: STX and CrtM Inhibition and Selectivity with Respect to hSQS.

| | IC₅₀ | IC₅₀ | IC₅₀ | |
|---|---|---|---|---|
| | (STX) | (CrtM) | (hSQS) | Relative |
| Compound | (μM) | (μM) | (μM) | selectivity^{a} |
| 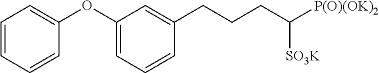 BPH-652 (FX24B-04-652) | 0.11 | 7.9 | 0.023 | 1 |
| 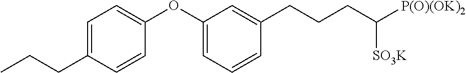 BPH-701 (FX29-08-701) | 0.10 | 2.2 | 1.9 | 295 |
| 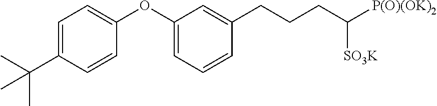 BPH-760 (FX40-19-760) | 0.021 | 10.5 | 4.0 | 132 |
| 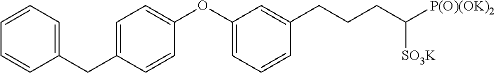 BPH-769 (FX26-05-769) | 0.049 | 13.5 | 0.72 | 19 |

^{a}Selectivity [IC₅₀(hSQS)/IC₅₀(CrtM)] with respect to that of BPH-652 (FX24B-04-652).

### Example 2

### Phosphonoacetohydroxamate and Phosphonoacetamide Inhibitors of Staphyloxanthin Biosynthesis in Staphylococcus aureus

In this Example, we disclose a new class of inhibitors of S. aureus CrtM, based on rational, structure-based drug design. The protein crystal structures of CrtM, together with a substrate analog farnesyl thiodiphosphate and BPH-652 (FX24B-04-652), show that both negatively charged headgroups, thiodiphosphate and phosphonosulfonate, bind/chelate two magnesium ions (Mg²⁺) at the active site of the protein. Similar results have also been observed for related bisphosphonate inhibitors in the active site of farnesyl and geranylgeranyl diphosphate synthases (FPPS and GGPPS). This suggests that the ability of the head group to bind/chelate Mg²⁺ is an important feature and can be exploited to design a new generation of CrtM inhibitors. We therefore designed a series of phosphonoacetohydroxamate and phosphonoacetamide compounds, such as BPH-808 (FX2-808) and BPH-830 (FX6-830). The rationale is that since hydroxamate and amide groups are known to be able to coordinate to Mg²⁺, phosphonoacetohydroxamate and phosphonoacetamide groups should be able to chelate/bind multiple Mg²⁺ ions strongly, and these compounds should represent novel CrtM inhibitors. We made these two compounds and tested their activities against CrtM (enzyme) as well as staphyloxanthin (STX) biosynthesis in S. aureus (cell activity). It turned out that these two compounds are very potent inhibitors in both assays. BPH-808 (FX2-808) had an IC₅₀ value of 67.5 μM against CrtM, about eight times less active than BPH-652 (FX24B-04-652). However, it showed the same cellular activity (IC₅₀(STX)=0.11 μM), due presumably to enhanced cell uptake. BPH-830 (FX6-830) is even more potent, with excellent CrtM activity (IC₅₀=8.9 μM). More importantly, its cell activity against STX was about fourteen times better than BPH-652 (FX24B-04-652). We now have made a series of analogous compounds, including BPH-808 (FX2-808) and BPH-830 (FX6-830), and their enzyme and cell activities are shown below (including BPH-652 (FX24B-04-652) as a reference). These compounds and their IC₅₀(STX) levels are given in Table 2, below.

TABLE 2
Phosphonoacetohydroxamate and Phosphonoacetamide Inhibition of STX.

| | | IC₅₀(CrtM) | IC₅₀(STX) |
|---|---|---|---|
| Compound No. | Structure | (μM) | (μM) |
| 652 (FX24B-04-652) | 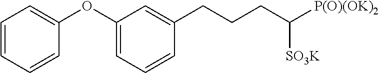 | 7.9 | 0.11 |
| 808 (FX2-808) | 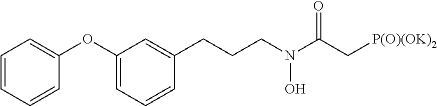 | 67.5 | 0.108 |
| 809 (FX3-809) | 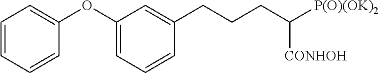 | 859 | 4.23 |
| 810 (FX4-810) | 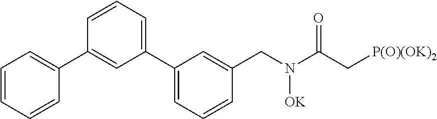 | 597 | 158 |
| 826 (FX5-826) | 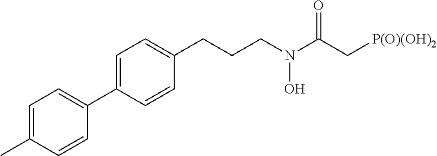 | >1500 | 4.11 |
| 830 (FX6-830) | 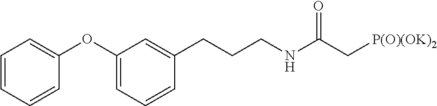 | 8.9 | 0.008 |
| 839 (FX7-839) | 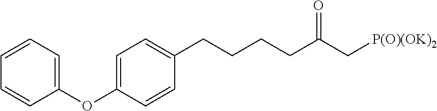 | 1520 | 28 |
| 841 (FX8-841) | 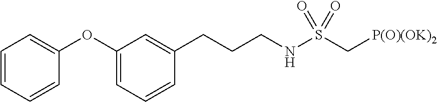 | 1640 | >500 |
| 846 (FX9-846) | 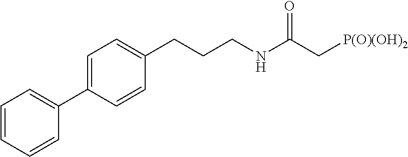 | 165 | 5.0 |
| 847 (FX10-847) | 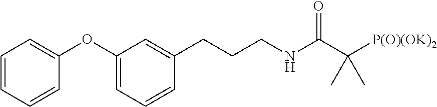 | 204 | >500 |
| 848 (FX11-848) | 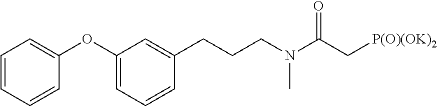 | 193 | 8.6 |
| 884 (FX12-884) | 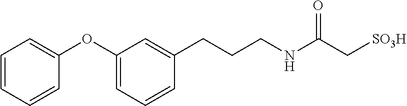 | 171 | 15 |
| 885 (FX13-885) | 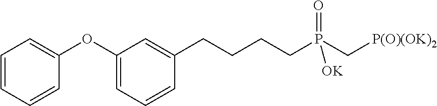 | 45 | 0.44 |
| 886 (FX14-886) | 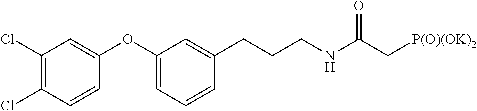 | 7.0 | 0.040 |
| 887 (FX15-887) | 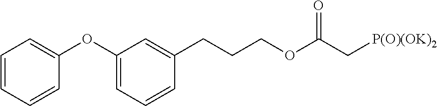 | 1190 | 1.5 |
| 888 (FX16-888) | 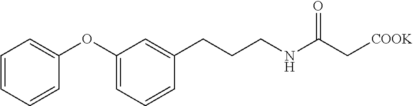 | >1500 | 200 |
| 897 (FX17-897) | 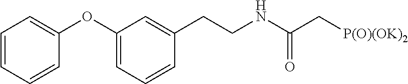 | 323 | 3.0 |
| 898 (FX18-898) | 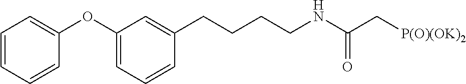 | 1080 | 35 |
| 926 (FX19-926) | 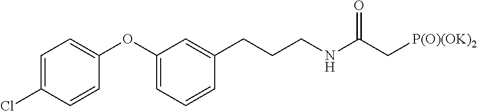 | 15.3 | 0.028 |
| 927 (FX20-927) | 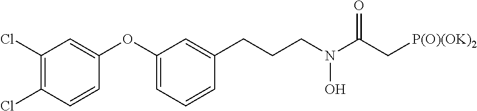 | 67.2 | 0.042 |

These new types of backbone structures are of particular interest since they may not have the plasma-binding drawback of the compound, BPH-652 (FX24B-04-652). These compounds may also have important activity in inhibiting cell membrane raft-associated activity of importance in viral replication (e.g. HCV/HIV), in cancer (many signaling proteins bind to cholesterol containing rafts), and potentially in Alzheimer's disease (prevent formation of amyloid (31-42).

### Example 3

### Phosphonosulfonates are Potent, Selective Inhibitors of Dehydrosqualene Synthase and Staphyloxanthin Biosynthesis in Staphylococcus aureus

Staphylococcus aureus is a major human pathogen, producing a wide spectrum of clinically significant hospital- and community-acquired infections. Methicillin-resistant strains of S. aureus (MRSA) have now reached epidemic proportions, and pose a significant challenge to the public health. A recent CDC study has shown that more people in the United States die from invasive MRSA each year than do from HIV/AIDS (1, 2). There is, therefore, an urgent need to find new therapies. One unconventional approach to anti-infective therapy involves blocking bacterial virulence factors (3), a potential benefit of this strategy being that, without the “life or death” selective pressure exerted by classical antibiotics, bacteria may be less prone to develop drug resistance.

An important virulence factor of S. aureus is the golden carotenoid pigment, staphyloxanthin (STX), whose numerous double bonds can react with, and thus deactivate, the reactive oxygen species (ROS) generated by neutrophils and macrophages, making S. aureus resistant to innate immune clearance (4, 5). STX has been shown to be essential for infectivity: bacteria that lack staphyloxanthin are nonpigmented, are susceptible to neutrophil killing, and fail to produce disease in a mouse skin and systemic infection models (4, 6). STX biosynthesis is thus a novel target for preventing or treating S. aureus infections. The first committed step in STX biosynthesis is catalyzed by the enzyme dehydrosqualene synthase, also called diapophytoene synthase or CrtM, and involves the head-to-head condensation of two molecules of farnesyl diphosphate (FPP) to produce the C30 species, presqualene diphosphate, which is then converted to dehydrosqualene (FIG. 1) (5). Since this condensation is remarkably similar to the first step in mammalian cholesterol biosynthesis (FIG. 1), we reasoned that known squalene synthase inhibitors, developed in the context of cholesterol-lowering therapy, might also inhibit dehydrosqualene synthase. This turns out to be the case and we recently reported that phosphonosulfonates such as 1 (BPH-652 (FX24B-04-652) or rac-BMS-187745), developed by Bristol-Myers Squibb and advanced through phase I/II human clinical trials (7, 8), potently inhibit S. aureus CrtM, as well as STX biosynthesis in the bacterium (6). Upon treatment with 1 (FX24B-04-652), the resulting non-pigmented S. aureus are much more susceptible to killing by hydrogen peroxide, and are less able to survive in freshly isolated human whole blood than are normally pigmented S. aureus. Moreover, in an in vivo systemic S. aureus infection model, the bacterial counts in kidneys of mice treated with 1 (FX24B-04-652) were reduced by 98%, compared to those of a control group. These results show that 1 (FX24B-04-652) represents a novel lead compound for virulence factor-based therapy of S. aureus infection. Here, we report the synthesis and testing of a library of 38 phosphonosulfonates and related bisphosphonates against CrtM, against STX biosynthesis in S. aureus and, as a counterscreen, against an expressed human squalene synthase. We report both qualitative as well as quantitative (QSAR, quantitative structure activity relationships) results for CrtM and STX biosynthesis inhibition, as well as CrtM/SQS selectivity. In addition, we investigate how cell activity can be predicted from enzyme (CrtM) inhibition results, opening the way to the further development of potent and selective inhibitors of S. aureus virulence.

### Qualitative Structure-Activity Relationships in CrtM Inhibition

The inhibitor class we investigate here originated in early research aimed at developing human squalene synthase inhibitors as cholesterol lowering drugs. Early SQS inhibitors were based on the structure of FPP, the substrate for SQS, with the labile diphosphate group being replaced by a bisphosphonate. However, isoprenyl sidechains were metabolized in vivo. This could be overcome by incorporation of e.g. a biphenyl isostere (e.g., 2 in Table 3) (9):

TABLE 3
Human Squalene Synthase Inhibitors 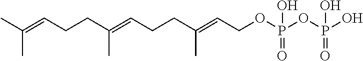
FPP 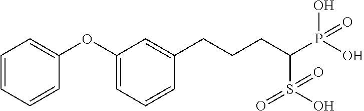
1 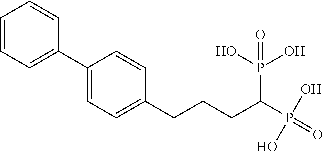
2

Referring to Table 3, these compounds potently bound to bone, in addition to causing elevation in liver enzymes. Phosphonosulfonates, on the other hand, bound only weakly to bone, and the diphenyl ether phosphonosulfonates (such as 1) were not broken down and did not cause elevated liver function (10). Plus, in our previous work we found that 1 had promising activity against CrtM as well as cellular and in vivo activity (6). In the present study, we sought to develop more potent CrtM inhibitors with improved activity in S. aureus cells that, at the same time, have poor activity against human SQS, reducing formation of the 1,10-dioic acid FPP metabolite that is formed as a result of SQS inhibition. We first synthesized a small library of five diphenyl ether phosphonosulfonates, five biphenyl phosphonosulfonates and three biphenyl bisphosphonates, based in part on the types of compound tested previously as SQS inhibitors, and examined them for their activity against CrtM.

The structures and IC₅₀ values (in parentheses) in CrtM inhibition of these compounds are shown in Table 6. Bisphosphonates 2 (FX65-02) and 3 (FX43-3) are the most potent CrtM inhibitors (IC₅₀=0.5 and 0.2 μM, or Ki=˜1 and 0.5 nM), being an order of magnitude more active than are any of the phosphonosulfonates. However, these compounds were found to have only modest activity in inhibition of STX biosynthesis in S. aureus (see below), due perhaps to poor cell uptake. Compound 4 (FX44-4, IC₅₀=5.4 μM), an analogous phosphinophosphonate compound, was ˜10× less active than was 2 (FX65-02), showing similar activity in CrtM inhibition as the phosphonosulfonates, which are likely to have the same formal charge. For the phosphonosulfonates with diphenylether sidechains, 5 (FX23-01) and 6 (FX29-08-701), which contain parasubstituted fluoro and n-propyl groups, respectively, were ˜4× as active (IC₅₀=2.3 and 2.2 μM, or Ki=˜5 nM) as 1 (FX24B-04-652). Compound 7 (FX34-13, IC₅₀=7.2 μM), containing a paratrifluoromethyl substituent, showed similar activity to that found with 1, while 8 (FX45-8), which contains an ortho-benzyl group, was ˜5× less active than 1 (FX24B-04-652, IC₅₀=37.2 μM). The phosphonosulfonates containing biphenyl sidechains, 9 (FX30-09), 10 (FX47-10), 11 (FX31-10), 12 (FX49-12), and 13 (FX50-13) had, in general, significantly reduced activities.

These results, together with the bacterial cell-based results discussed below, are of interest since they show that the biphenyl bisphosphonates are the most potent CrtM inhibitors, with IC₅₀ values of <1 μM. They do, however, have poor cell-based activities, with IC₅₀>1 μM and consequently, were not selected for further development. The biphenyl phosphonosulfonates, on the other hand, had generally poor activity against CrtM (average IC₅₀ for the five compounds investigated ˜50 μM), making them also less attractive candidates for development. However, the diphenyl ether phosphonosulfonates had, on average, an IC₅₀ value of −11 μM (or a Ki of ˜30 nM), plus, these compounds were very potent in cell based assays (i.e. in inhibiting STX biosynthesis by S. aureus), as discussed in detail below, and were thus selected for further development.

To see if major improvements in CrtM activity might be obtained by modifying the phosphonosulfonate sidechain, we next synthesized the eight analogs of 1 (FX24B-04-652), shown in Table 6, in which we modified the sidechain heteroatom (O→NH, CH₂ and a carbazole); the position of the heteroatom (meta→para); the length of the alkyl sidechain (N=1, 2 and 3 CH₂ groups), plus, we synthesized the R and S-enantomers of 1 (FX24B-04-652), since previously the S-form was found to be far more potent than the R-form, in inhibiting SQS (11). The optically active (S)-1 (FX24A-02, IC₅₀=1.4 μM) was ˜30× more active than its (R)-enantiomer (FX42-R1) in CrtM inhibition (Table 6). Shorter linkers between the aromatic ring and phosphonosulfonate headgroup, as found in 14 (FX51-14) and 15 (FX52-15), led to essentially no CrtM activity (IC₅₀>500 μM). As for compounds 16 (FX36-15, IC₅₀=43.7 μM) and 17 (FX37-16, IC₅₀=20.4 μM), replacing the bridging —O— atoms with —NH— and —CH2-, respectively, resulted in reduced CrtM inhibition activity. The 4-phenoxyphenyl analog 18 (FX39-18) was slightly more active than its 3-phenoxyphenyl counterpart 1 (FX24B-04-652) in CrtM inhibition but was less active in the cell based assay, while compound 19 (FX56-19), a fused tricyclic analog, had very poor activity against CrtM (IC₅₀=170 μM). These results, together with those discussed above, clearly indicated that investigating additional diphenyl ether phosphonosulfonates would be of interest.

We therefore next synthesized 17 more substituted phosphonosulfonates whose structures and activities in CrtM inhibition are shown in Table 4, together with the previously described results for 1 (FX24B-04-652), 5 (FX23-01), 6 (FX29-08-701), and 7 (FX34-13). The following structure-activity relationship features can be seen from these results: (i) Diphenyl ether phosphonosulfonates substituted with F— or —CF₃ had diminished activity when the substituent was in the 2′-position relative to the corresponding 3′- or 4′-substituted analogs; (ii) Halogen-containing groups, including F, CF₃ and Cl, generally enhance activity; (iii) Alkyl groups with various shapes and sizes located at the 4′-position result in modest activity changes when compared to 1 (FX24B-04-652), while oxygen-containing groups at the same position significantly decrease activity; (iv) Di-halogen substituted compounds are more active than the parent compound, 1 (FX24B-04-652); and (v) Substitution at the 6-position with F has little effect on activity but incorporation of an OMe group in this location abrogates most activity. Clearly, the above represents a complex set of empirical observations that require a more quantitative analysis.

TABLE 4
IC₅₀ values of Diphenylether Phosphonosulfonates in CrtM Inhibition

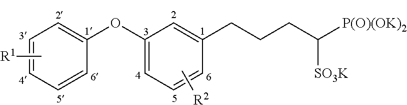

| | | | | CrtM IC₅₀ |
|---|---|---|---|---|
| Entry | Compound | R¹ | R² | values (μM) |
| 1 | 1 (FX24B-04-652, | H | H | 7.9 |
| | BPH-652) | | | |
| 2 | 5 (FX23-01, | 4′-F | H | 2.3 |
| | BPH-771) | | | |
| 3 | 20 (FX35-14, | 3′-F | H | 2.2 |
| | BPH-783) | | | |
| 4 | 21 (FX28-07, | 2′-F | H | 6.9 |
| | BPH-784) | | | |
| 5 | 7 (FX34-13, | 4′-CF₃ | H | 7.2 |
| | BPH-775) | | | |
| 6 | 22 (FX38-17, | 3′-CF₃ | H | 9.8 |
| | BPH-756) | | | |
| 7 | 23 (FX64-23, | 2′-CF₃ | H | 53.7 |
| | BPH-786) | | | |
| 8 | 24 (FX33-12, | 4′-Cl | H | 2.5 |
| | BPH-761) | | | |
| 9 | 6 (FX29-08-701, | 4′-n-propyl | H | 2.2 |
| | BPH-701) | | | |
| 10 | 25 (FX40-19-760, | 4′-tert-butyl | H | 10.5 |
| | BPH-760) | | | |
| 11 | 26 (FX26-05-769, | 4′-CH₂Ph | H | 13.5 |
| | BPH-769) | | | |
| 12 | 27 (FX57-27, | 4′-OH | H | 28.2 |
| | BPH-801) | | | |
| 13 | 28 (FX58-28, | 4′-OPh | H | 169.8 |
| | BPH-802) | | | |
| 14 | 29 (FX59-29, | 4′-(furan-2-yl) | H | 26.3 |
| | BPH-807) | | | |
| 15 | 30 (FX25-03, | 3′, 4′-2F | H | 1.7 |
| | BPH-785) | | | |
| 16 | 31 (FX60-31, | 3′, 4′-2Cl | H | 2.2 |
| | BPH-762) | | | |
| 17 | 32 (FX61-32, BPH-803) | 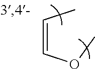 | H | 2.1 |
| 18 | 33 (FX32-11, | 3′, 5′-2F | H | 4.6 |
| | BPH-772) | | | |
| 19 | 34 (FX62-34, | 3′, 5′-2Cl | H | 6.3 |
| | BPH-753) | | | |
| 20 | 35 (FX27-06, | 4′-F | 6-F | 2.1 |
| | BPH-787) | | | |
| 21 | 36 (FX63-36, | 4′-F | 6-OCH₃ | 81.3 |
| | BPH-788) | | | |

### Quantitative Structure-Activity Relationships in CrtM Inhibition

To investigate the structure-activity relationships outlined above, we carried out a comparative molecular similarity indices analysis (CoMSIA) (12) of the CrtM inhibition activities of these phosphonosulfonate compounds. All molecules were constructed and minimized using the MMFF94x force field in the program MOE (13). Compounds were then aligned using the flexible alignment module (14) in MOE, which perceives common features within the molecules (e.g., similar partial charge, H-bond acceptor, aromaticity, hydrophobicity, etc.). We used fully deprotonated phosphonosulfonate head groups, based on the observation that 1 (FX24B-04-652) binds two Mg²⁺ in the CrtM active site (6), and our previous NMR and quantum chemical studies on bisphosphonates, which indicate deprotonation when bisphosphonates bind to Mg²⁺ (15). All compounds were constructed with the (S)-configuration, based on the observation that (S)-1 (FX24A-02) is far more active than is its (R)-enantiomer (FX42-R1) in CrtM inhibition (as also found in SQS inhibition (10)). The aligned compounds were exported into Sybyl (16), then we used a partial least-squares (PLS) method to regress the CrtM inhibitory activity and CoMSIA field data. The 3-D QSAR model yielded r2=0.98, q2 (no. of components)=0.72 (7), F-test=245.8, and a pIC₅₀ error of 0.12, as shown in Table 5. To further validate the model, we performed five leave-five-out training/test sets, obtaining on average a factor of 1.8× error between predicted and experimental IC₅₀ values (Table 4) There is a relatively large hydrophobic contribution (46.6%) to the CoMSIA model, followed by steric (25.2%), electrostatic (19.9%) and H-bond donor (8.3%) interactions. The CoMSIA results are in good agreement with the more qualitative experimental observations: steric and hydrophobic-favorable areas at the 3′- and 4′-positions correspond to generally increased activities of 1 (FX24B-04-652) analogs with para- and meta-substituents on the distal phenyl ring. Two large steric penalty areas at the 4- and 6-positions of the proximal phenyl ring account for the decreased activity of the biphenyl phosphonosulfonates 9 (FX30-09), 10 (FX47-10), 11 (FX31-10), 12 (FX49-12), and 13 (FX50-13) in Table 6 and 36 (FX63-36), entry 21 in Table 4. In addition, a positive-charge-favored region within the distal phenyl ring helps account for the enhanced activity of the (electron-withdrawing) halogen containing phosphonosulfonates, e.g., 5 (FX23-01), 24 (FX33-12) and 30 (FX25-03). The negative-charge favored area obviously correlates with the activity of bisphosphonates 2 (FX65-02) and 3 (FX43-3) in CrtM inhibition, since the sulfonate group is replaced by the more negatively-charged phosphonate group.

TABLE 5
CoMSIA Results for CrtM Inhibition

| | | CrtM enzyme | | | | | | | | |
|---|---|---|---|---|---|---|---|---|---|---|
| | | experimental | | | | | | | | |
| | | activity | | CoMSIA pIC₅₀ predictions | | | | | | |
| | | IC₅₀ | pIC₅₀ | training | | test sets^{a} | | | | |
| compound | | (μM) | (M) | set | residual | 1 | 2 | 3 | 4 | 5 |
| 3 | (FX43-3) | 0.18 | 6.74 | 6.61 | 0.13 | 6.54 | 6.53 | 6.53 | 6.49 | 6.58 |
| 2 | (FX64-02) | 0.48 | 6.32 | 6.55 | −0.23 | 6.35 | 6.41 | 6.19 | 6.29 | 6.49 |
| 30 | (FX25-03) | 1.7 | 5.76 | 5.62 | 0.14 | 5.61 | 5.60 | 5.56 | 5.55 | 5.70 |
| 35 | (FX27-06) | 2.1 | 5.68 | 5.69 | −0.01 | 5.69 | 5.41 | 5.51 | 5.58 | 5.76 |
| 32 | (FX61-32) | 2.1 | 5.67 | 5.69 | −0.02 | 5.65 | 5.46 | 5.44 | 5.54 | 5.49 |
| 6 | (FX29-08-701) | 2.2 | 5.66 | 5.64 | 0.02 | 5.82 | 5.12 | 5.60 | 5.63 | 5.61 |
| 20 | (FX35-14) | 2.2 | 5.66 | 5.34 | 0.32 | 5.33 | 5.32 | 5.29 | 5.25 | 5.41 |
| 31 | (FX60-31) | 2.2 | 5.65 | 5.62 | 0.03 | 5.77 | 5.90 | 5.97 | 5.88 | 5.69 |
| 5 | (FX23-01) | 2.3 | 5.63 | 5.44 | 0.19 | 5.45 | 5.34 | 5.32 | 5.37 | 5.48 |
| 24 | (FX33-12) | 2.5 | 5.6 | 5.46 | 0.14 | 5.61 | 5.49 | 5.54 | 5.52 | 5.51 |
| 33 | (FX32-11) | 4.6 | 5.34 | 5.37 | −0.03 | 5.35 | 5.38 | 5.36 | 5.28 | 5.44 |
| 4 | (FX44-4) | 5.4 | 5.27 | 5.21 | 0.06 | 5.09 | 5.13 | 4.87 | 5.06 | 5.23 |
| 18 | (FX39-18) | 5.8 | 5.24 | 5.34 | −0.10 | 5.28 | 5.10 | 5.20 | 5.22 | 5.32 |
| 34 | (FX62-34) | 6.3 | 5.2 | 5.21 | −0.01 | 5.24 | 5.47 | 5.54 | 5.40 | 5.23 |
| 21 | (FX28-07) | 6.9 | 5.16 | 5.18 | −0.02 | 5.16 | 5.01 | 5.03 | 5.04 | 5.25 |
| 7 | (FX34-13) | 7.2 | 5.14 | 5.30 | −0.16 | 5.59 | 5.33 | 5.51 | 5.47 | 5.32 |
| 1 | (FX24B-04-652) | 7.9 | 5.1 | 5.15 | −0.05 | 5.15 | 5.05 | 5.03 | 5.05 | 5.18 |
| 10 | (FX47-10) | 9.1 | 5.04 | 4.92 | 0.12 | 4.94 | 4.99 | 5.04 | 4.94 | 4.88 |
| 22 | (FX38-17) | 9.8 | 5.01 | 5.05 | −0.04 | 5.23 | 5.28 | 5.32 | 5.43 | 5.05 |
| 25 | (FX40-19-760) | 10.5 | 4.98 | 5.10 | −0.12 | 5.37 | 4.74 | 5.18 | 5.15 | 4.95 |
| 26 | (FX26-05-769) | 13.5 | 4.87 | 4.91 | −0.04 | 4.86 | 5.03 | 4.67 | 4.69 | 4.83 |
| 17 | (FX37-16) | 20.4 | 4.69 | 4.72 | −0.02 | 4.89 | 4.81 | 4.82 | 4.81 | 4.67 |
| 9 | (FX30-09) | 21.9 | 4.66 | 4.73 | −0.07 | 4.79 | 4.79 | 4.85 | 4.77 | 4.68 |
| 11 | (FX31-10) | 24.5 | 4.61 | 4.75 | −0.14 | 4.89 | 4.80 | 5.03 | 4.82 | 4.65 |
| 29 | (FX59-29) | 26.3 | 4.58 | 4.71 | −0.13 | 4.81 | 4.44 | 4.66 | 4.57 | 4.61 |
| 27 | (FX57-27) | 28.2 | 4.55 | 4.55 | 0.00 | 4.55 | 4.55 | 4.55 | 4.55 | 4.55 |
| 8 | (FX45-8) | 37.2 | 4.43 | 4.43 | 0.00 | 4.35 | 4.31 | 4.27 | 4.34 | 4.68 |
| 16 | (FX36-15) | 43.7 | 4.36 | 4.36 | 0.00 | 4.36 | 4.36 | 4.80 | 4.36 | 4.36 |
| 23 | (FX64-23) | 53.7 | 4.27 | 4.23 | 0.04 | 4.28 | 4.37 | 4.32 | 4.33 | 4.24 |
| 12 | (FX49-12) | 72.4 | 4.14 | 4.15 | −0.01 | 4.10 | 3.72 | 4.06 | 3.89 | 4.08 |
| 36 | (FX63-36) | 81.3 | 4.09 | 4.16 | −0.07 | 4.28 | 4.48 | 4.46 | 4.50 | 4.15 |
| 13 | (FX50-13) | 123 | 3.91 | 3.86 | 0.05 | 3.91 | 4.16 | 4.08 | 4.12 | 3.84 |
| 19 | (FX56-19) | 170 | 3.77 | 3.79 | −0.02 | 3.91 | 4.09 | 3.92 | 4.07 | 3.70 |
| 28 | (FX58-28) | 170 | 3.77 | 3.80 | −0.03 | 3.90 | 3.85 | 3.96 | 4.20 | 3.82 |
| 15 | (FX52-15) | 500 | 3.3 | 3.33 | −0.03 | 3.24 | 3.25 | 3.12 | 3.20 | 3.40 |
| 14 | (FX51-14) | 1700 | 2.77 | 2.65 | 0.12 | 2.53 | 2.65 | 2.50 | 2.56 | 2.71 |
| | | | r² | 0.98 | | 0.97 | 0.94 | 0.95 | 0.95 | 0.99 |
| | | | q² | 0.72 | | 0.69 | 0.71 | 0.72 | 0.67 | 0.70 |
| | | | F-test | 245.8 | | 151.6 | 82.4 | 113.9 | 97.5 | 280.1 |
| | | | N | 7 | | 6 | 5 | 4 | 5 | 7 |
| | | | n | 36 | | 31 | 31 | 31 | 31 | 31 |

^{a}Bold values epresent predicted activities of compounds that were not included in the training set.

TABLE 6
Phosphonosulphonate Compounds and Their CrtM IC₅₀ Levels 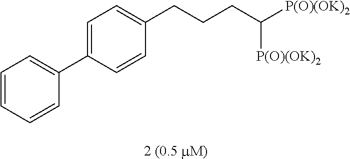
(FX65-02) 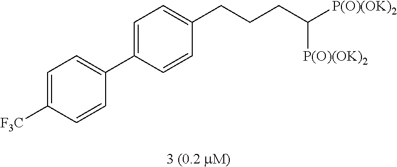
(FX43-3) 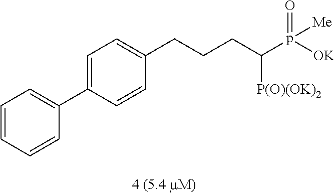
(FX44-4) 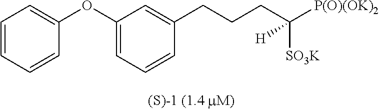
(FX24A-02) 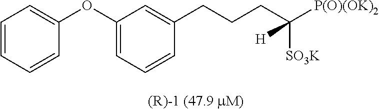
(FX42-R1) 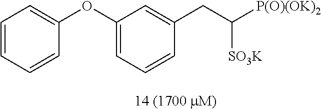
(FX51-14, BPH-755) 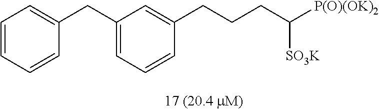
(FX37-16) 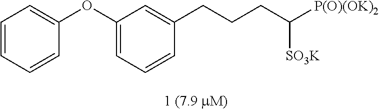
(FX24B-04-652) 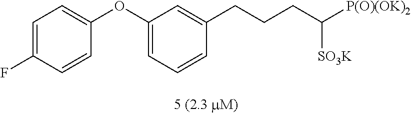
(FX23-01) 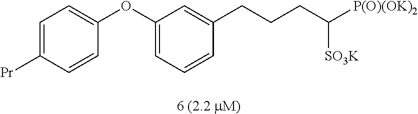
(FX29-08-701) 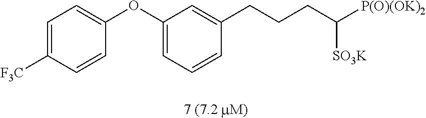
(FX34-13) 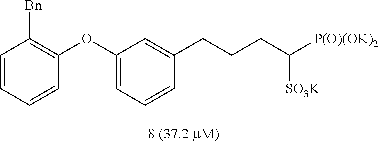
(FX45-8) 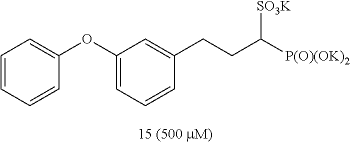
(FX52-15) 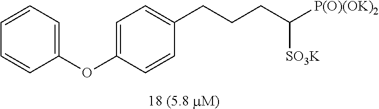
(FX39-18) 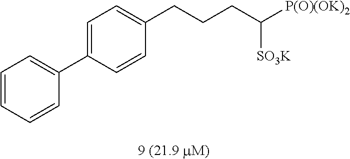
(FX30-09, BPH-700) 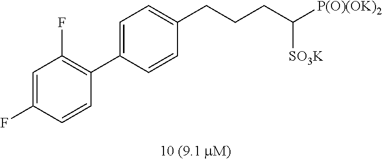
(FX47-10) 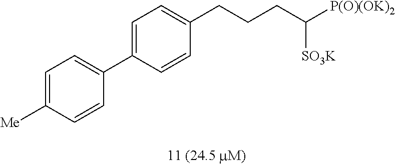
(FX31-10) 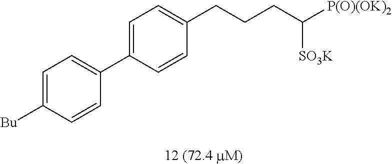
(FX49-12, BPH-698) 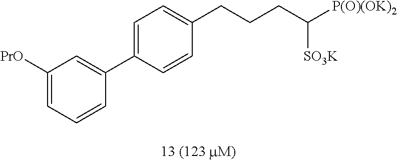
(FX50-13) 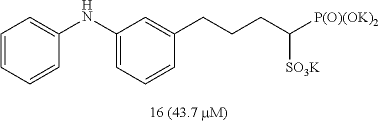
(FX36-15) 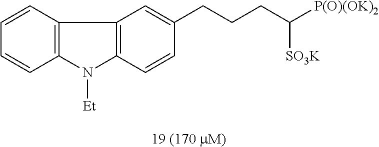
(FX56-19)

### Staphyloxanthin Biosynthesis Inhibition

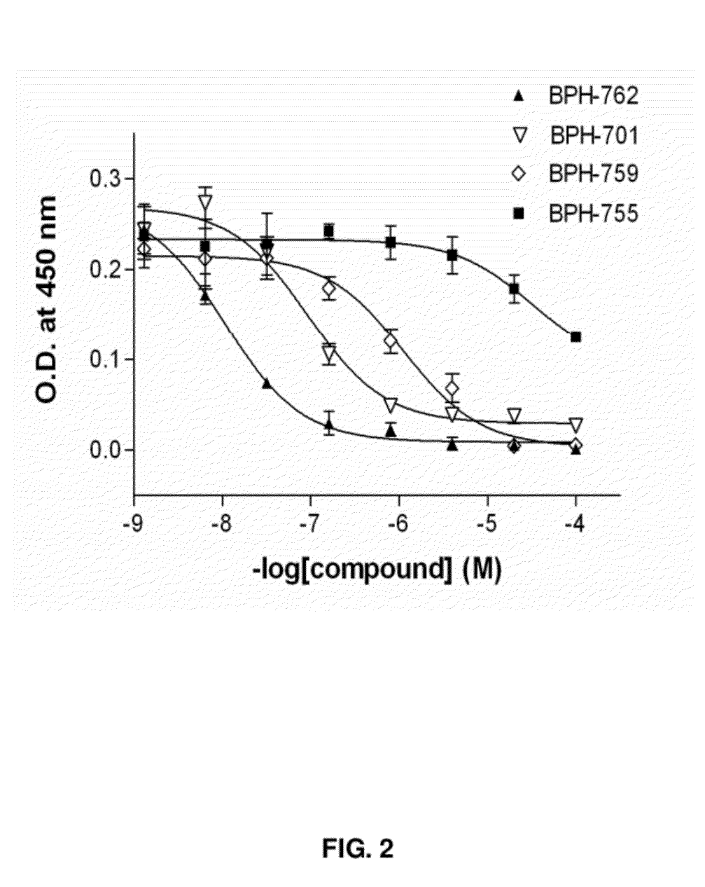

The results described above are of interest since they indicate that dehydrosqualene (CrtM) can be potently inhibited by phosphonosulfonates, and by bisphosphonates. The question next arises: how potent are these compounds in inhibiting STX biosynthesis in S. aureus? We thus treated S. aureus bacteria with serially diluted compounds at 37° C. for 3 days, after which the STX pigment was extracted with methanol. Optical densities were measured at 450 nm and IC₅₀ values for inhibition of pigment formation for each compound were calculated using a standard dose-response curve. The rank ordered IC₅₀ values of the 38 compounds investigated are shown in Table 7 and dose-response curves of representative compounds are shown in FIG. 2. Surprisingly, we found that STX biosynthesis inhibition in S. aureus was poorly correlated with CrtM (enzyme) inhibition, with an R²=0.27 for the CrtM/STX inhibition pIC₅₀ values.

TABLE 7
Staphyloxanthin inhibition in S. aureus

| | Staphyloxanthin |
|---|---|
| Compound | IC₅₀ (μM) |

| | | |
|---|---|---|
| 31 | (FX60-31) | 0.011 |
| 33 | (FX32-11) | 0.015 |
| 32 | (FX61-32) | 0.015 |
| 8 | (FX45-8) | 0.019 |
| 25 | (FX40-19-760) | 0.021 |
| 24 | (FX33-12) | 0.039 |
| 7 | (FX34-13) | 0.040 |
| 26 | (FX26-05-769) | 0.049 |
| (S)-1 | (FX24A-02) | 0.050 |
| 34 | (FX62-34) | 0.051 |
| 5 | (FX23-01) | 0.052 |
| 35 | (FX27-06) | 0.059 |
| 22 | (FX38-17) | 0.074 |
| 6 | (FX29-08-701) | 0.093 |
| 28 | (FX58-28) | 0.10 |
| 1 | (FX24B-04-652) | 0.11 |
| 30 | (FX25-03) | 0.11 |
| 12 | (FX49-12) | 0.16 |
| 17 | (FX37-16) | 0.17 |
| 11 | (FX31-10) | 0.18 |
| 10 | (FX47-10) | 0.18 |
| 18 | (FX39-18) | 0.19 |
| 20 | (FX35-14) | 0.22 |
| (R)-1 | (FX42-R1) | 0.31 |
| 9 | (FX30-09) | 0.35 |
| 23 | (FX64-23) | 0.46 |
| 13 | (FX50-13) | 0.49 |
| 16 | (FX36-15) | 0.50 |
| 21 | (FX28-07) | 0.51 |
| 3 | (FX43-3) | 1.1 |
| 29 | (FX59-29) | 1.1 |
| 4 | (FX44-4) | 1.4 |
| 2 | (FX65-02) | 2.2 |
| 27 | (FX57-27) | 6.8 |
| 36 | (FX63-36) | 13.8 |
| 15 | (FX52-15) | 20.4 |
| 14 | (FX51-14) | 33.9 |
| 19 | (FX56-19) | 263 |

Presumably, this poor correlation is due to the fact that no consideration of cell permeability or drug uptake is involved in the in vitro enzyme inhibition data. To try to take this into account, we therefore chose to use Slog P (the logarithm of the octanol/water partition coefficient (17)) to describe this effect by using the following equation:

pIC₅₀(STX,cell)=a·pIC₅₀(CrtM)+b·S log P+c (EQ 1)

where a, b and c are regression coefficients from a linear regression analysis. This yielded an R²=0.60 for the experimental-versus-predicted pIC₅₀ values or an R²=0.53 for a leave-two-out (L2O) prediction test set, to be compared with R²=0.16 for a L2O test set of predictions using solely the enzyme pIC₅₀ results. Of course, there is no a priori reason to use S log P as the extra descriptor, or to limit the method to use of a single extra descriptor. So we next used a three descriptor model:

pIC₅₀(STX,cell)=a·pIC₅₀(CrtM)+b·B+c·C+d

where B, C are all possible descriptor pairs available in MOE that have non-Boolean values (i.e. the properties do not contain 0's or 1's). The top 10 “enzyme plus two descriptor” search results are shown in Table S1, rank ordered by R² value.

TABLE S1
Top ten “enzyme plus 2-descriptor” combinations with their coefficients
and relative contributions for the S. aureus cell pIC₅₀ predictions.

| | | Relative | Relative | Relative |
|---|---|---|---|---|
| S. aureus (CrtM) | | Importance of | Importance of | Importance of |
| pIC₅₀ (cell) = | R² | pIC₅₀(enzyme) | Descriptor B^{a} | Descriptor C^{a} |
| 1.07935 | 0.71715 | 1.000000 | 0.498511 | 0.929362 |
| +1.07168 * crtm_new | | | | |
| +0.01232 * PEOE_VSA-1 | | | | |
| +0.38370 * E_stb | | | | |
| −0.51520 | 0.70532 | 1.000000 | 0.489720 | 0.773848 |
| +0.93203 * crtm_new | | | | |
| +0.01378 * vsa_hyd | | | | |
| +0.27786 * E_stb | | | | |
| 0.88476 | 0.70334 | 1.000000 | 0.533638 | 0.746758 |
| +0.86354 * crtm_new | | | | |
| −0.46499 * logS | | | | |
| +0.24843 * E_stb | | | | |
| −1.83200 | 0.70285 | 1.000000 | 0.486128 | 0.750599 |
| +0.93216 * crtm_new | | | | |
| +0.01654 * | | | | |
| PEOE_VSA_NEG | | | | |
| +0.26955 * E_stb | | | | |
| 1.32345 | 0.70124 | 1.000000 | 0.509204 | 0.701953 |
| +0.89953 * crtm_new | | | | |
| +0.52931 * logP(o/w) | | | | |
| +0.24326 * E_stb | | | | |
| 0.66970 | 0.69410 | 1.000000 | 0.734455 | 0.546206 |
| +0.94974 * crtm_new | | | | |
| +0.00704 * E_sol | | | | |
| +0.01894 * | | | | |
| PEOE_VSA_NEG | | | | |
| −2.44204 | 0.69302 | 1.000000 | 0.700314 | 0.762682 |
| +0.91088 * crtm_new | | | | |
| +0.02328 * | | | | |
| PEOE_VSA_NEG | | | | |
| −0.24100 * E_str | | | | |
| −2.28190 | 0.69268 | 1.000000 | 0.484585 | 0.731641 |
| +0.90675 * crtm_new | | | | |
| +0.00778 * ASA | | | | |
| +0.25558 * E_stb | | | | |
| −0.53109 | 0.69117 | 1.000000 | 0.706822 | 0.792306 |
| +0.90728 * crtm_new | | | | |
| +0.01936 * vsa_hyd | | | | |
| −0.24937 * E_str | | | | |
| −0.50061 | 0.68972 | 1.000000 | 0.429537 | 0.742886 |
| +1.01675 *crtm_new | | | | |
| +0.34050 * chi1_C | | | | |
| +0.29099 * E_stb | | | | |

Each of the top 10 results contains pIC₅₀ (CrtM) as the major descriptor, and the R² value obtained for the top solution, R²=0.72, is clearly an improvement over that obtained using solely CrtM or CrtM and S log P results. We then used a leave two out method to produce a test set result in which we recomputed all training sets minus the two compounds of interest, then used the coefficients and descriptors to predict the two omitted compounds. In this way, the activity of each compound was predicted 37 times. The R² in this leave two out test set was R²=0.62, a major improvement over the R²=0.16 using solely enzyme inhibition data (and the same leave two out test set approach). To verify that this predictivity did not occur by chance, we repeated the leave two out process, using scrambled cell activity data. This process was repeated 10 times, with the average R² values (leave two out, scrambled) being R²=0.10 (Table S1). Clearly, then, phosphonosulfonates are potent inhibitors of the CrtM enzyme, and their activity can be relatively well predicted by using the combinatorial descriptor search method, even when the cell/enzyme data is very poorly correlated. Moreover, as might be expected based on the enzyme inhibition results (Table 6), (S)-1 (FX24A-02) is far more active than is (R)-1 (FX42-R1), in cells (Table 7).

### Selectivity of CrtM Inhibition

Since phosphonosulfonates, such as 1 (FX24B-04-652), were originally developed as inhibitors of squalene synthase in the context of cholesterol-lowering therapy (7, 8, 10), we were also interested to see how they inhibit human SQS, since SQS inhibition results in formation of a 1,10-dioic acid metabolite (from unused FPP). We thus screened each of the compounds described above for their activity against an expressed human SQS enzyme. Results are shown in Table 8. We find that CrtM inhibition is moderately correlated with human SQS inhibition, R2=0.50, consistent with the modest sequence homology of S. aureus CrtM and human SQS (30% identity, 36% similarity) (6). This modest correlation reflects something that is potentially beneficial: that some good CrtM inhibitors are poor hSQS inhibitors. Consider for example the 4′-n-propyl species 6 (FX29-08-701) in Table 4. This compound has a 2.2 μM IC₅₀ versus CrtM and a 1.9 μM IC₅₀ versus hSQS, Table 8, and is therefore a much poorer hSQS inhibitor than is the parent compound, 1 (FX24B-04-652). For compound 1 (FX24B-04-652) (used previously in vivo (6)), the CrtM IC₅₀ is 7.9 μM but 1 (FX24B-04-652) is a very potent hSQS inhibitor with an IC₅₀ of 23 nM, Table 8. The results shown in Table 8 are rank ordered in terms of selectivity for CrtM over hSQS inhibition or IC₅₀(hSQS)/IC₅₀(CrtM), such that a larger number means more CrtM selectivity, and are also given in terms of selectivity relative to 1 (FX24B-04-652), that is selectivity of compound/selectivity of 1 (FX24B-04-652). The compound with the highest relative selectivity is thus 6 (FX29-08-701), which is (1.9/2.2)+(0.023/7.9) or ˜300× more selective a CrtM inhibitor than is 1 (FX24B-04-652). Remarkably, both 1 (FX24B-04-652) and 6 (FX29-08-701) have, however, very similar IC₅₀ values for STX biosynthesis (Table 7): 110 nM for 1 (FX24B-04-652) and 93 nM for 6 (FX29-08-701). These results strongly suggest that it is possible to make potent inhibitors of STX biosynthesis, such as 6 (FX29-08-701), that have much better relative selectivity in inhibiting SQS than does 1 (FX24B-04-652).

TABLE 8
CoMSIA Results for CrtM/hSQS Selectivity

| | | Experimental data | | | | | | | | | | |
|---|---|---|---|---|---|---|---|---|---|---|---|---|
| | | | | | pIC50 | | | | | | | |
| | | | | relative | (CrtM) − | | | | | | | |
| | | hSQS | CrtM/ | CrtM/ | pIC50 | CoMSIA predictions | | | | | | |
| | | IC₅₀ | hSQS | hSQS | (hSQS) | training | | test sets | | | | |
| compound | | (μM) | selectivity^{a} | selectivity | (M) | set | residual | 1 | 2 | 3 | 4 | 5 |
| 6 | (FX29-08-701) | 1.9 | 0.85 | 295 | −0.07 | −0.16 | 0.09 | −0.17 | −0.18 | −0.18 | −0.16 | −0.17 |
| 25 | (FX40-19-760) | 4.0 | 0.38 | 132 | −0.42 | −0.38 | −0.04 | −0.28 | −0.26 | −0.31 | −0.43 | −0.27 |
| 19 | (FX56-19) | 11.7 | 0.068 | 24.0 | −1.16 | −0.99 | −0.17 | −1.13 | −1.15 | −1.17 | −0.97 | −1.13 |
| 26 | (FX26-05-769) | 0.72 | 0.054 | 18.6 | −1.27 | −1.29 | 0.02 | −1.41 | −1.40 | −1.43 | −1.29 | −1.42 |
| 7 | (FX34-13) | 0.17 | 0.024 | 8.3 | −1.62 | −1.52 | −0.10 | −1.51 | −1.49 | −1.53 | −1.52 | −1.51 |
| 10 | (FX47-10) | 0.22 | 0.024 | 8.3 | −1.62 | −1.52 | −0.10 | −1.60 | −1.45 | −1.60 | −1.51 | −1.89 |
| 29 | (FX59-29) | 0.54 | 0.021 | 7.1 | −1.69 | −1.70 | 0.01 | −1.75 | −1.73 | −1.77 | −1.71 | −1.71 |
| 30 | (FX25-03) | 0.033 | 0.019 | 6.6 | −1.72 | −2.12 | 0.40 | −2.22 | −2.21 | −2.38 | −2.09 | −2.22 |
| 27 | (FX57-27) | 0.47 | 0.016 | 5.8 | −1.78 | −2.34 | 0.56 | −2.37 | −2.39 | −2.44 | −2.34 | −2.40 |
| 9 | (FX30-09) | 0.35 | 0.016 | 5.6 | −1.79 | −1.98 | 0.19 | −2.01 | −1.81 | −1.90 | −2.00 | −2.02 |
| 13 | (FX50-13) | 1.8 | 0.015 | 5.1 | −1.83 | −1.93 | 0.10 | −2.04 | −2.01 | −2.00 | −1.90 | −2.02 |
| 3 | (FX43-3) | 0.003 | 0.014 | 4.8 | −1.86 | −2.00 | 0.14 | −1.84 | −1.80 | −1.82 | −1.99 | −1.88 |
| 4 | (FX44-4) | 0.072 | 0.014 | 4.7 | −1.87 | −1.93 | 0.06 | −1.74 | −1.71 | −1.75 | −1.97 | −1.70 |
| 11 | (FX31-10) | 0.29 | 0.012 | 4.1 | −1.93 | −1.64 | −0.29 | −1.62 | −1.39 | −1.50 | −1.63 | −1.64 |
| 36 | (FX63-36) | 0.79 | 0.0098 | 3.4 | −2.01 | −1.88 | −0.13 | −1.97 | −1.99 | −2.00 | −1.86 | −1.95 |
| 24 | (FX33-12) | 0.020 | 0.0079 | 2.8 | −2.10 | −2.15 | 0.05 | −2.12 | −2.12 | −2.18 | −2.09 | −2.13 |
| 18 | (FX39-18) | 0.042 | 0.0072 | 2.5 | −2.14 | −2.23 | 0.09 | −2.30 | −2.32 | −2.30 | −2.27 | −2.38 |
| 5 | (FX23-01) | 0.017 | 0.0071 | 2.5 | −2.15 | −2.29 | 0.14 | −2.32 | −2.33 | −2.42 | −2.29 | −2.34 |
| 33 | (FX32-11) | 0.032 | 0.0071 | 2.5 | −2.15 | −2.20 | 0.05 | −2.33 | −2.28 | −2.34 | −2.19 | −2.30 |
| 22 | (FX38-17) | 0.069 | 0.0071 | 2.5 | −2.15 | −2.31 | 0.16 | −2.31 | −2.31 | −2.29 | −2.47 | −2.30 |
| 28 | (FX58-28) | 1.1 | 0.0065 | 2.2 | −2.19 | −2.12 | −0.07 | −2.15 | −2.16 | −2.16 | −2.12 | −2.14 |
| 31 | (FX60-31) | 0.014 | 0.0062 | 2.1 | −2.21 | −2.08 | −0.13 | −2.08 | −2.06 | −2.16 | −1.99 | −2.05 |
| 20 | (FX35-14) | 0.008 | 0.0036 | 1.3 | −2.44 | −2.31 | −0.13 | −2.38 | −2.35 | −2.45 | −2.31 | −2.34 |
| 17 | (FX37-16) | 0.069 | 0.0034 | 1.2 | −2.47 | −2.63 | 0.16 | −2.54 | −2.47 | −2.47 | −2.55 | −2.56 |
| 23 | (FX64-23) | 0.18 | 0.0033 | 1.1 | −2.48 | −2.48 | 0.00 | −2.47 | −2.49 | −2.47 | −2.53 | −2.87 |
| 34 | (FX62-34) | 0.020 | 0.0032 | 1.1 | −2.49 | −2.43 | −0.06 | −2.47 | −2.40 | −2.34 | −2.41 | −2.40 |
| 1 | (FX24B-04-652) | 0.023 | 0.0029 | 1.0 | −2.54 | −2.50 | −0.04 | −2.50 | −2.49 | −2.50 | −2.52 | −2.48 |
| 14 | (FX51-14) | 3.7 | 0.0022 | 0.8 | −2.66 | −2.50 | −0.16 | −2.56 | −2.58 | −2.63 | −2.51 | −2.52 |
| 2 | (FX65-02) | 0.001 | 0.0019 | 0.6 | −2.73 | −2.84 | 0.11 | −2.66 | −2.62 | −2.62 | −2.90 | −2.60 |
| 35 | (FX27-06) | 0.004 | 0.0018 | 0.6 | −2.74 | −2.65 | −0.09 | −2.62 | −2.62 | −2.67 | −2.66 | −2.61 |
| 16 | (FX36-15) | 0.079 | 0.0018 | 0.6 | −2.74 | −2.68 | −0.06 | −2.78 | −2.83 | −2.77 | −2.72 | −2.82 |
| 12 | (FX49-12) | 0.13 | 0.0018 | 0.6 | −2.74 | −2.70 | −0.04 | −2.74 | −2.72 | −2.76 | −2.68 | −2.74 |
| 32 | (FX61-32) | 0.002 | 0.0011 | 0.4 | −2.96 | −2.60 | −0.36 | −2.51 | −2.53 | −2.55 | −2.66 | −2.55 |
| 15 | (FX52-15) | 0.55 | 0.0011 | 0.4 | −2.96 | −2.92 | −0.04 | −2.91 | −2.96 | −2.96 | −2.93 | −2.89 |
| 21 | (FX28-07) | 0.005 | 0.00073 | 0.3 | −3.14 | −2.78 | −0.36 | −2.75 | −2.74 | −2.77 | −2.81 | −2.74 |
| 8 | (FX45-8) | 0.003 | 7.1E−05 | 0.02 | −4.15 | −4.16 | 0.01 | −4.17 | −4.16 | −4.16 | −4.18 | −4.18 |
| | | | | | r² | 0.94 | | 0.93 | 0.93 | 0.94 | 0.95 | 0.92 |
| | | | | | q² | 0.54 | | 0.50 | 0.52 | 0.51 | 0.55 | 0.51 |
| | | | | | F-test | 76.5 | | 71.6 | 75.6 | 82.5 | 75.0 | 64.5 |
| | | | | | N | 6 | | 5 | 5 | 5 | 6 | 5 |
| | | | | | n | 36 | | 33 | 33 | 33 | 33 | 33 |

^{a}Bold values represent predicted activities of compounds that were not included in the training set.

To put these observations on a more structural basis, we carried out a QSAR/CoMSIA analysis, using the relative selectivity results. Using the same compound alignment as above, we obtained r²=0.94, q2=0.54 (6), F-test=76.5, and a pIC₅₀ error of 0.20, as shown in Table 8. We also validated this model by using five leave-three-out training/test sets, finding on average a factor of 1.8× error between predicted and experimental IC₅₀ values (Table 8). There is a large hydrophobic contribution (54.8%) to the CoMSIA field results, followed by steric (29.5%) and electrostatic (15.7%) fields. The CoMSIA fields show that overlapping steric and hydrophobic field features near the 4′-position are favored for CrtM selectivity. Hydrophobic and a steric disfavored regions are located near the 2′- and 3′-positions, respectively, and are responsible for the poor selectivity of compounds such as 8 (FX45-8) and 21 (FX28-07).

### Conclusions

The results described above are of interest for a number of reasons. First, we made 38 phosphonosulfonate and bisphosphonate compounds and investigated their activity in inhibiting S. aureus dehydrosqualene synthase (CrtM), the enzyme involved in the first committed step in the biosynthesis of the virulence factor, staphyloxanthin, in S. aureus. The most active compounds were bisphosphonates, but they had poor activity in cells, while many phosphonosulfonates were active in both enzyme and cell assays. Second, we developed a QSAR model for CrtM inhibition that had good predicitivity. Third, we determined the activity of each compound against staphyloxanthin biosynthesis, in S. aureus. The pIC₅₀ results were poorly correlated (R²=0.27, training; R²=0.16, test set) with the enzyme inhibition pIC₅₀ values. However, using a combinatorial descriptor search, we obtained significant improvements in cell activity predictions with R²=0.72 (training) and R²=0.62 (test set) results. Fourth, we investigated the inhibition of human squalene synthase by these compounds, and used a QSAR method to enable good predictions of CrtM/SQS selectivity, relative to that exhibited by 1 (FX24B-04-652). Several CrtM inhibitors, e.g., 6 (FX29-08-701) and 25 (FX40-19-760), also showed very potent activity in bacterial cell-based assays. Overall, these results are of general interest since they demonstrate that diphenylether phosphonosulfonates are potent inhibitors of dehydrosqualene synthase and of the STX biosynthesis in S. aureus and as such, they have considerable potential for further development in a new approach to combating S. aureus infections.

### Chemistry: General Aspects

General Synthesis Methods

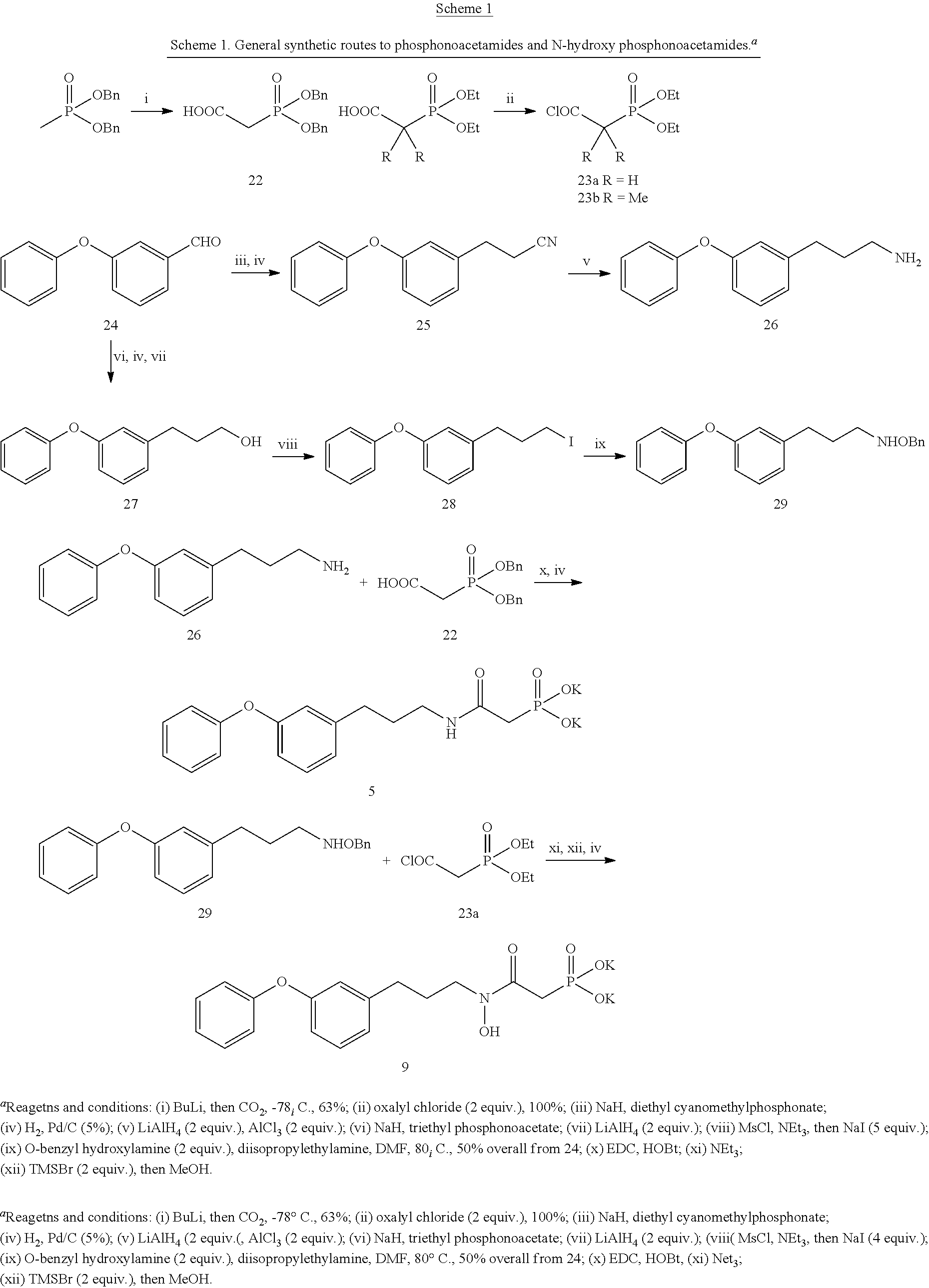

Referring to Scheme 1, the reaction of aldehyde 24 and sodium diethyl cyanomethylphosphonate in THF gave, after hydrogenation, compound 25 in almost quantitative yield, which after reduction with 2 equiv. of LiAlH4 and AlCl₃ afforded amine 26. Amine 26 was then reacted with dibenzyl phosphonoacetic acid 22 in the presence of the coupling reagent N-ethyl-N′-(3-dimethylaminopropyl)-carbodiimide (EDC) to give, after hydrogenation to remove the benzyl groups, a phosphonoacetamide (e.g., 5). N-hydroxyphosphonoacetamide compounds, such as 9, were prepared from substituted hydroxylamine 29 and diethylphosphonoacetyl chloride 23, after hydrolysis with TMSBr (to remove ethyl phosphono-esters) and hydrogenation (to remove O-benzyl protecting group), also shown in Scheme 1. Compounds 13, 11, 17, 21, 14 and 16 were made similarly, with a carbodiimide mediated amide/ester formation reaction as the main step, as shown in Scheme 2:

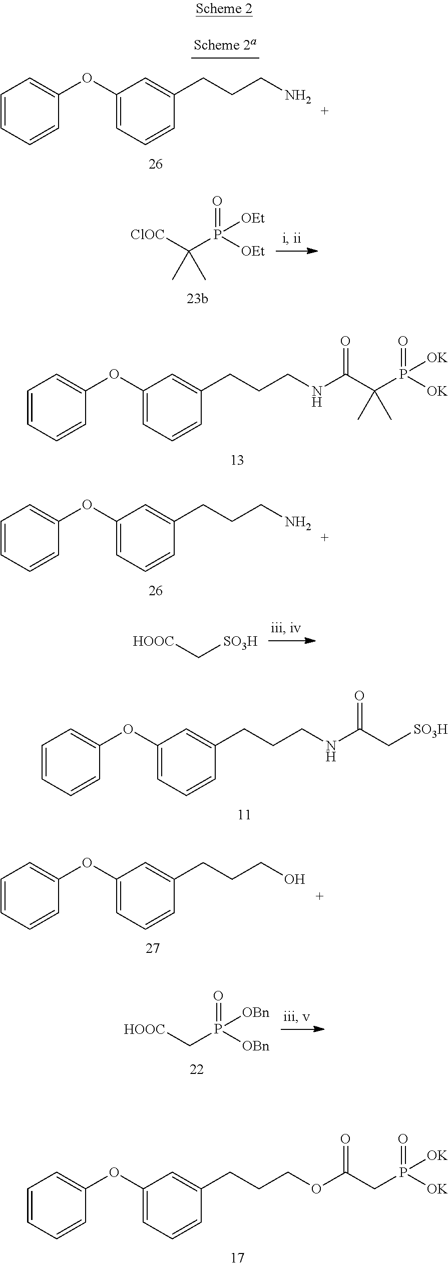

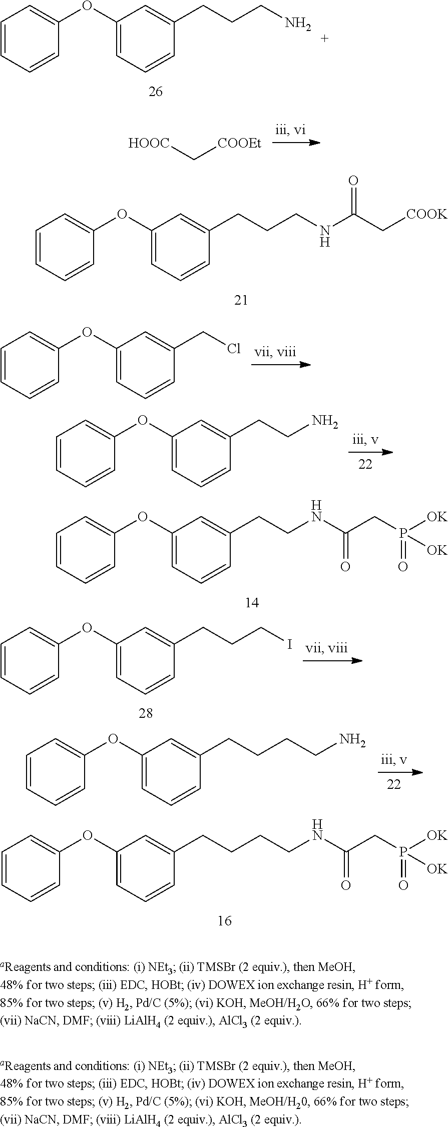

Other compounds were made as outlined in Scheme 3. Compound 15 is an analog of 5, but has a hydroxamate group in its side chain. Alkylation of ethyl dibenzylphosphonoacetate with iodide 28 gave compound 30, as shown in Scheme 3. The carboxylate ethyl ester was then selectively hydrolyzed (1M KOH) and the corresponding acid coupled with O-benzyl hydroxylamine under standard carbodiimide conditions, followed by hydrogenation to remove three benzyl protecting groups, to give 15. Compound 18 is a β-ketophosphonate analog of 5 and its synthesis began with a Suzuki coupling reaction of an ethyl 4-pentenoate derived boron compound and 4-bromodiphenylether (Scheme 3), affording carboxylate ester 31. Compound 31 was then reacted with 2 equivalent of lithium diethyl methylphosphonate at −78° C. to give, after TMSBr mediated hydrolysis, 18. The reaction of precursor amine 26 with methanesulfonyl chloride produced methylsulfamide 32 (Scheme 3). This was then treated with 2 equivalent of butyl lithium to give a dianion, which was reacted with diethyl chlorophosphate and hydrolyzed with TMSBr, resulting in the phosphono-sulfamide 19. Compound 7 was made by an alkylation reaction of the dianion of triethyl methylphosphinomethylphosphonate with iodide 28, followed by hydrolysis.

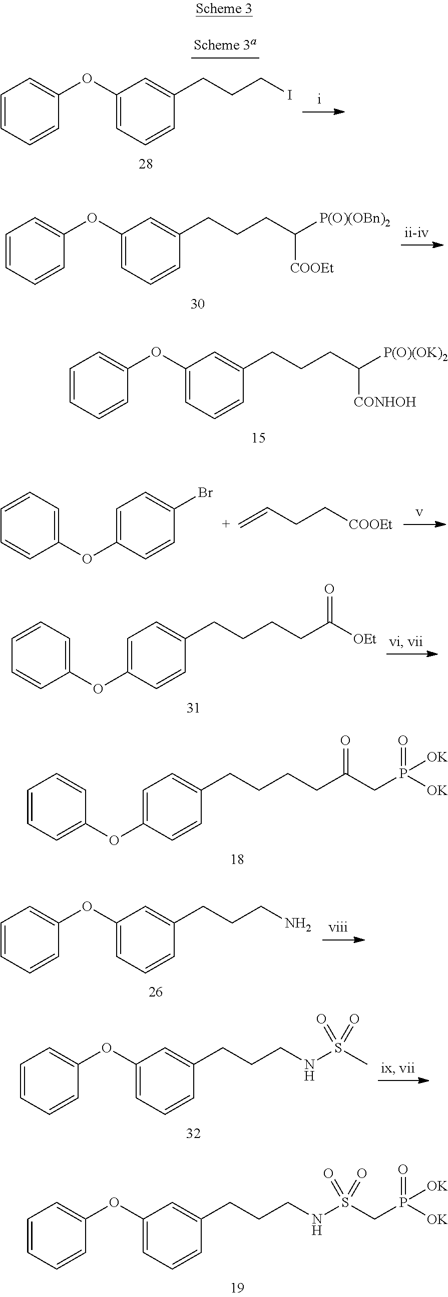

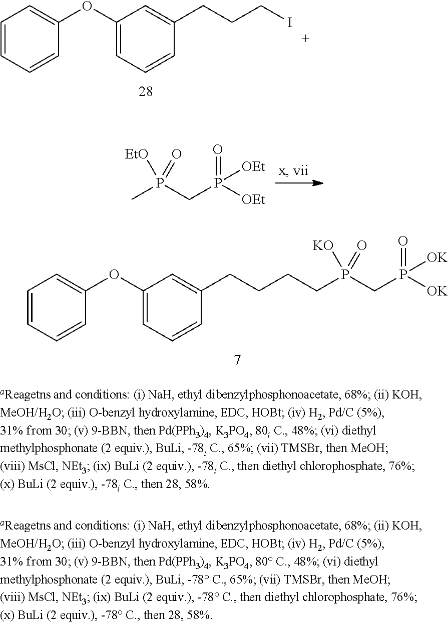

General method A. Triethyl phosphonoacetate, or diethyl cyanomethylphosphonate (3.3 mmol) was added dropwise to NaH (145 mg, 60% in oil, 3.6 mmol) suspended in dry THF (7 mL) at 0° C. To the resulting clear solution was added a benzaldehyde (3 mmol) and, after stirring at room temperature for 0.5 h, the reaction mixture was partitioned between diethyl ether (50 mL) and water (50 mL). The organic layer was dried and evaporated. The oily residue was then hydrogenated in MeOH (15 mL) in the presence of 5% Pd/C (50 mg). The catalyst was filtered and the filtrate concentrated and dried in vacuo.

General method B. The nitrile (or ester) obtained using general method A was added slowly to 2 equiv. of LiAlH4/AlCl3, or LiAlH4, in dry THF at 0° C. After stirring at room temperature for 2 h, the reaction was carefully quenched by adding a few drops of water, and the reaction mixture filtered and evaporated.

General method C. To a solution of a carboxylic acid (1 mmol) and an amine (1 mmol) in CH₂Cl₂ (5 mL) were added N-ethyl-N′-(3-dimethylaminopropyl)-carbodiimide (EDC) (1.5 mmol) and 1-hydroxybenzotriazole (1 mmol). After stirring for 2 h at room temperature, 50 mL of ethyl acetate was added and the reaction mixture washed successively with 1N HCl (5 mL), water (5 mL) and saturated NaHCO3 (5 mL), dried, and evaporated. The amide was purified using flash chromatography (silica gel; ethyl acetate).

General method D. A DMF solution (3 mL) containing a halide (3 mmol), O-benzyl hydroxylamine (6 mmol) and diisopropylethylamine (3 mmol) was heated at 90° C. for 24 h. After cooling, diethyl ether (50 mL) was added and the mixture was washed with H2O (20 mL), dried, and evaporated. The alkylated hydroxylamine, such as 29, was purified by using column chromatography (silica gel; hexane/ethyl acetate 6/1).

General method E. To a diethyl phosphonate (1 mmol) in dry CH™₃CN (3 mL) was added TMSBr (2 mmol) at room temperature. After 6 h, the solution was evaporated and methanol (5 mL) added. Neutralization with 1 N KOH to pH=8, followed by evaporation to dryness and triturating with acetone, gave a white powder.

Diethyl phosphonoacetyl chloride (23a). Compound 23a was prepared by mixing diethyl phosphonoacetic acid (1.5 mmol) with oxalyl chloride (3 mmol) in benzene (5 mL) in the presence of one drop of DMF for 1 h, followed by evaporation. The oily residue was used immediately for the next reaction.

3-(3-phenoxyphenyl)-propyl iodide (28). Alcohol 27, obtained from 3-phenoxybenzaldehyde (3 mmol) following general methods A and B, in CH2Cl2 (10 mL) containing NEt3 (0.5 mL, 3.6 mmol) was reacted with methane sulfonyl chloride (230 μL, 3 mmol) at 0° C. After 1 h, diethyl ether (50 mL) and water (50 mL) were added and the organic layer collected, washed with 1 N HCl and saturated NaHCO3, dried, and evaporated to dryness. The oily residue was treated with NaI (1.35 g, 9 mmol) in acetone (7 mL) at 60° C. for 1 h. The reaction mixture was then partitioned between diethyl ether (50 mL) and water (50 mL) and the organic layer washed with 5% Na2S2O3, dried, and evaporated to dryness to give iodide 28. The iodide thus obtained is quite pure, according to 1H and ¹³C NMR spectra, and may be used in the next step without further purification.

N-[3-(3-(3,4-dichlorophenoxy)phenyl)-propyl]-phosphonoacetamide dipotassium salt (4). 3-(3-(3,4-Dichlorophenoxy)phenyl)-propylamine was prepared from 3-(3,4-dichlorophenoxy)benzaldehyde (1 mmol), using general method A, and was then coupled with dibenzylphosphonoacetic acid according to general method C to give the dibenzyl ester of 4. The benzyl groups were removed by catalytic hydrogenation (5% Pd/C in methanol for 1 h) followed by neutralization with KOH to give compound 4 as a white powder (245 mg, 48% overall yield). Anal. (C17H16Cl2K2NO5P.0.5CH3OH) C, H, N; 1H NMR (400 MHz, D2O): δ 1.60-1.70 (m, 2H, CH2); 2.35 (d, J=20 Hz, 2H, CH2P); 2.46 (t, J=7.6 Hz, 2H, PhCH2); 2.98 (t, J=7.2 Hz, 2H, CH2N); 6.70-7.30 (m, 7H, aromatic); 31P NMR (D2O): δ 13.6.

N-[3-(3-phenoxyphenyl)-propyl]-phosphonoacetamide dipotassium salt (5). Amine 26 was prepared from 3-phenoxybenzaldehyde (1 mmol) using general method A, and was then coupled with dibenzyl phosphonoacetic acid according to general method C, to give the dibenzyl ester of 5. The benzyl groups were removed by hydrogenation for 1 hr, catalyzed with 5% Pd/C in methanol, followed by neutralization with KOH to give compound 5 as a white powder (307 mg, 62% overall yield). Anal. (C17H18K2NO5P.1.5H2O) C, H, N; 1H NMR (400 MHz, D2O): δ 1.60-1.70 (m, 2H, CH2); 2.35 (d, J=20 Hz, 2H, CH2P); 2.46 (t, J=7.6 Hz, 2H, PhCH2); 2.98 (t, J=7.2 Hz, 2H, CH2N); 6.70-7.30 (m, 9H, aromatic); 31P NMR (D2O): δ 13.7.

N-[3-(3-(4-chlorophenoxy)phenyl)-propyl]-phosphonoacetamide dipotassium salt (6). 6 was prepared in the same way as 5, but using 3-(4-chlorophenoxy)benzaldehyde (1 mmol) as starting material, as a white powder (267 mg, 58% overall yield). Anal. (C17H17ClK2NO5P.0.3KCl.1.5H2O) C, H, N; 1H NMR (400 MHz, D2O): δ 1.60-1.70 (m, 2H, CH2); 2.37 (d, J=20 Hz, 2H, CH2P); 2.49 (t, J=7.6 Hz, 2H, PhCH2); 3.01 (t, J=7.2 Hz, 2H, CH2N); 6.70-7.30 (m, 8H, aromatic); 31P NMR (D2O): δ 13.5.

3-(3-Phenoxyphenyl)-propylphosphinyl-methylphosphonic acid tripotassium salt (7). Triethyl methylphosphinylmethylphosphonate (1 mmol) was treated with BuLi (2.2 mmol) in THF at −78° C. for 1 h, followed by addition of iodide 28 (1.1 mmol). The reaction mixture was allowed to warm to room temperature over 3 h and was then quenched with saturated NH4Cl. The product was purified with column chromatography (silica gel; ethyl acetate/methanol:20/1) and deprotected using general method E to give 7 as a white powder (320 mg, 62% overall yield). Anal. (C17H19K3O6P2.H2O) C, H; 1H NMR (400 MHz, D2O): δ 1.30-1.60 (m, 6H, 3CH2); 1.75-1.85 (m, 2H, CH2P); 2.43 (t, J=7.6 Hz, 2H, PhCH2); 6.70-7.30 (m, 9H, aromatic); 31P NMR (D2O): δ 16.3 (s, 1P); 39.9 (s, 1P).

N-Hydroxy-N-[3-(3-(3,4-dichlorophenoxy)phenyl)-propyl]-phosphonoacetamide dipotassium salt (8). 8 was prepared in the same way as 9, but using 3-(3,4-dichlorophenoxy)benzaldehyde (3 mmol) as starting material, as a white powder (428 mg, 28% overall yield). Anal. (C17H16Cl2K2NO6P.KBr) C, H, N; 1H NMR (400 MHz, D2O): δ 1.75-1.85 (m, 2H, CH2); 2.48 (t, J=7.6 Hz, 2H, PhCH2); 2.63 (d, J=20 Hz, 2H, CH2P); 3.46 (t, J=7.2 Hz, 2H, CH2N); 6.70-7.35 (m, 7H, aromatic); 31P NMR (D2O): δ 15.6.

N-Hydroxy-N-[3-(3-phenoxyphenyl)-propyl]-phosphonoacetamide dipotassium salt (9). General method D with iodide 28 gave substituted hydroxylamine 29 (1 mmol), which was reacted with the acid chloride in the presence of NEt3 (2 mmol) in CH₂Cl₂ (5 mL) at 0° C. After stirring for 1 h, the coupling product was purified by using column chromatography (silica gel; ethyl acetate), and was then deprotected following general method E. Hydrogenation (5% Pd/C, MeOH) gave compound 9 as a white powder (312 mg, 56% overall yield). ¹H NMR (400 MHz, D2O): δ 1.65-1.75 (m, 2H, CH2); 2.42 (t, J=7.6 Hz, 2H, PhCH2); 2.66 (d, J=20 Hz, 2H, CH2P); 3.39 (t, J=7.2 Hz, 2H, CH2N); 6.70-7.20 (m, 9H, aromatic); 31P NMR (D2O): δ 15.8.

N-[3-(4-biphenyl)-propyl]-phosphonoacetamide (10). 3-(4-biphenyl)-propylamine was prepared from 4-phenylbenzaldehyde (1 mmol), using general method A, and was then coupled with dibenzyl phosphonoacetic acid according to general method C to give the dibenzyl ester of 10. The benzyl groups were removed by hydrogenation (catalyzed with 5% Pd/C in methanol) for 1 h, followed by neutralization with KOH, to give compound 10 as a white powder (222 mg, 65% overall yield). Anal. (C₁₇H2OK2NO4P.0.25 CH₃OH) C, H, N; 1H NMR (400 MHz, D2O): δ 1.65-1.75 (m, 2H, CH2); 2.31 (d, J=20 Hz, 2H, CH2P); 2.55 (t, J=7.6 Hz, 2H, PhCH2); 3.03 (t, J=7.2 Hz, 2H, CH2N); 7.20-7.55 (m, 9H, aromatic); 31P NMR (D2O): δ 12.8.

N-[3-(3-phenoxyphenyl)-propyl]-sulfoacetamide (11). Amine 26 (1 mmol) was coupled with sulfoacetic acid (1 mmol) according to general method C (without addition of 1-hydroxybenzotriazole) to give 11. The product was purified by using column chromatography (DOWEX ion exchange resin, H+ form, methanol as eluent) as an off-white powder (315 mg, 85% overall yield). Anal. (C17H19NO5S) C, H, N; 1H NMR (400 MHz, D2O): δ 1.60-1.70 (m, 2H, CH2); 2.44 (m, 2H, PhCH2); 3.02 (m, 2H, CH2N); 3.59 (s, 2H, CH2S); 6.70-7.30 (m, 9H, aromatic).

N-methyl-N-[3-(3-phenoxyphenyl)-propyl]-phosphonoacetamide dipotassium salt (12). Amine 26 (1 mmol) was reacted with benzyl chloroformate (ZCl, 1 mmol) in the presence of NEt3 to give Z-protected amine 26 which was then methylated in THF with MeI (1.5 equiv.) and NaH (1.2 equiv.) overnight. After hydrogenation (5% Pd/C in MeOH) to remove the Z-protecting group, the N-methylated amine 5 was coupled with dibenzyl phosphonoacetic acid, according to general method B, to give the dibenzyl ester of 12. The benzyl groups were removed by hydrogenation (5% Pd/C in methanol) for 1 h, followed by neutralization with KOH to give compound 12 as a white powder (220 mg, 50% overall yield). Anal. (C18H2OK2NO5P) C, H, N. The NMR spectrum of 12 showed that two rotamers (with respect to the amide bond) exist with ratio of ˜45:55. 1H NMR (400 MHz, D2O): δ 1.60-1.80 (m, 2H, CH2); 2.35-2.45 (m, 2H, CH2P); 2.45-2.95 (m, 5H, Me and PhCH2); 3.10-3.40 (m, 2H, CH2N); 6.80-7.30 (m, 9H, aromatic); 31P NMR (D2O): δ 13.6.

N-[3-(3-phenoxyphenyl)-propyl]-phosphonodimethylacetamide dipotassium salt (13). Diethyl phosphonodimethylacetate (3 mmol) was treated with 3 N KOH (5 mL) in ethanol (8 mL) for 24 h, followed by acidification with HCl to give the corresponding carboxylic acid. As with compound 23a, the acid was then converted to the acid chloride 23b which was reacted with 1 equiv. of amine 26 in the presence of NEt3 in CH₂Cl₂ (5 mL) at 0° C. After stirring for 1 h, the coupling product was purified by using column chromatography (silica gel; ethyl acetate), and was then deprotected following general method E to give 13 as a white powder (335 mg, 21% overall yield). Anal. (C19H22K2NO5P.0.5KBr.H2O) C, H, N; 1H NMR (400 MHz, D2O): δ 1.09 (d, J=13.6 Hz, 6H, 2CH3); 1.60-1.70 (m, 2H, CH2); 2.46 (m, 2H, PhCH2); 2.99 (m, 2H, CH2N); 6.85-7.25 (m, 9H, aromatic); 31P NMR (D2O): δ 22.9.

N-[2-(3-phenoxyphenyl)-ethyl]-phosphonoacetamide dipotassium salt (14). 3-Phenoxybenzyl chloride (2 mmol) and NaCN (2.2 mmol) were stirred in DMF (2 mL) overnight at 60° C. After cooling, diethyl ether (50 mL) was added and the mixture was washed with water and the organic layer dried and evaporated. 2-(3-phenoxyphenyl)-ethylamine was prepared from the nitrile so obtained, using general method B, and which was then coupled with dibenzyl phosphonoacetic acid according to general method C to give the dibenzyl ester of 14. The benzyl groups were removed by catalytic hydrogenation (5% Pd/C in methanol for 1 h) followed by neutralization with KOH to give compound 14 as a white powder (387 mg, 45% overall yield). Anal. (C16H16K2NO5P.H2O) C, H, N; 1H NMR (400 MHz, D2O): δ 2.39 (d, J=20 Hz, 2H, CH2P); 2.58 (t, J=7.6 Hz, 2H, PhCH2); 3.05 (t, J=7.2 Hz, 2H, CH2N); 6.70-7.30 (m, 9H, aromatic); ³¹P NMR (D2O): δ 13.8.

N-Hydroxy-2-phosphono-5-(3-phenoxyphenyl)-pentamide dipotassium salt (15). Iodide 28 was added to a cold DMF solution containing ethyl dibenzylphosphonoacetate (1 equiv.) and NaH (1.1 equiv.). After stirring for 3 h at room temperature, the product 30 was purified by using column chromatography (silica gel; hexane/ethyl acetate 1/1). 30 was then treated with 3 N KOH in EtOH/H₂O (3:1) for 24 h and the resulting solution was reduced in volume then acidified with 3 N HCl, to give the corresponding carboxylic acid. The acid so obtained was reacted with O-benzyl hydroxylamine, according to general method C, to give protected 15, which was then hydrogenated in the presence of 5% Pd/C in MeOH for 1 h to afford, after neutralization with KOH, 15, as a white powder (293 mg, 21% overall yield). Anal. (C17H18K2NO6P.0.5C2H50H) C, H, N; 1H NMR (400 MHz, D2O): δ 1.25-1.75 (m, 4H, CH2); 2.20-2.50 (m, 3H, CH+PhCH2); 6.70-7.25 (m, 9H, aromatic); 31P NMR (D2O): δ 17.5.

N-[4-(3-phenoxyphenyl)-butyl]-phosphonoacetamide dipotassium salt (16). Iodide 28 (2 mmol) and NaCN (2.2 mmol) were stirred in DMF (2 mL) overnight at 60° C., and after cooling diethyl ether (50 mL) was added and the mixture washed with water and the organic layer evaporated. 4-(3-phenoxyphenyl)-butylamine was prepared from the nitrile so obtained, using general method B, and was then coupled with dibenzyl phosphonoacetic acid, according to general method C to give the dibenzyl ester of 16. The benzyl groups were removed by catalytic hydrogenation (5% Pd/C in methanol for 1 h) followed by neutralization with KOH to give compound 16 as a white powder (475 mg, 51% overall yield). Anal. (C18H2OK2NO5P.1.5H2O) C, H, N; 1H NMR (400 MHz, D2O): δ 1.55-1.70 (m, 4H, CH2); 2.39 (d, J=20 Hz, 2H, CH2P); 2.48 (t, J=7.6 Hz, 2H, PhCH2); 3.01 (t, J=7.2 Hz, 2H, CH2N); 6.70-7.30 (m, 9H, aromatic); 31P NMR (D2O): δ 13.5.

3-(3-phenoxyphenyl)-propyl phosphonoacetate dipotassium salt (17). Alcohol 27 was coupled with dibenzylphosphonoacetic acid according to general method C to give dibenzyl ester of 17. The benzyl groups were removed by catalytic hydrogenation (5% Pd/C in methanol for 1 h) followed by neutralization with KOH to give compound 17 as a white powder (180 mg, 42% overall yield). Anal. (C17H17K2O6P) C, H; 1H NMR (400 MHz, D2O): δ 1.60-1.70 (m, 2H, CH2); 2.38 (d, J=20 Hz, 2H, CH2P); 2.44 (t, J=7.6 Hz, 2H, PhCH2); 3.47 (t, J=7.2 Hz, 2H, CH2O); 6.70-7.30 (m, 9H, aromatic); 31P NMR (D2O): δ 13.9.

2-Oxo-6-(4-phenoxyphenyl)-hexylphosphonic acid dipotassium salt (18). 9-BBN (0.5M in THF, 9 mL) was added to ethyl 4-pentenoate (3 mmol) at 0° C. and the reaction mixture stirred at room temperature for 2 h. 4-Bromodiphenylether (3 mmol), Pd(PPh3)4 (0.15 mmol), K3PO4 (6 mmol) and H2O (2 mL) were then added and the reaction mixture refluxed overnight. The organic layer was evaporated and purified by using column chromatography (silica gel; hexane/ether 6/1) to afford ester 31, which was then reacted with 2 equiv. of the lithium salt of diethyl methylphosphonate at −78° C., for 2 h. The reaction was quenched with saturated NH4Cl, diethyl ether added to extract the product, and the organic solvent removed. The oily residue was purified by using column chromatography (silica gel; ethyl acetate) and deprotected according to general method E, to give compound 18 as a white powder (312 mg, 20% overall yield). Anal. (C18H19K2O5P.0.5KBr.2H2O) C, H; 1H NMR (400 MHz, D2O): δ 1.30-1.50 (m, 4H, CH2); 2.44 (t, J=7.6 Hz, 2H, PhCH2), 2.54 (t, J=7.6 Hz, 2H, CH2CO); 2.70 (d, J=20 Hz, 2H, CH2P); 6.80-7.25 (m, 9H, aromatic); 31P NMR (D2O): δ 11.0.

N-[3-(3-phenoxyphenyl)-propyl]-phosphonomethylsufamide dipotassium salt (19). Amine 26 prepared from 3-phenoxybenzaldehyde (3 mmol) using general method A was reacted with 1 equiv. of methane sulfonyl chloride in CH₂Cl₂ in the presence of 1.2 equiv. of NEt3 at 0° C. After 1 h, 50 mL of ethyl acetate was added and the reaction mixture was washed successively with 1 N HCl, water, NaHCO3, then dried and evaporated. The oily residue was treated with 2.2 equiv. of BuLi at −78° C. for 1 h followed by addition of 0.6 equiv. of diethyl chlorophosphate. The reaction mixture was warmed to 0° C. over 1 h then quenched with saturated NH4Cl. Column chromatography (silica gel; ethyl acetate), followed by hydrolysis using general method E gave 19 as a white powder (366 mg, 42% overall yield). Anal. (C16H18K2NO6P.KBr) C, H, N; 1H NMR (400 MHz, D2O): δ 1.60-1.75 (m, 2H, CH2); 2.50 (t, J=7.6 Hz, 2H, PhCH2); 2.87 (t, J=7.2 Hz, 2H, CH2N); 1.18 (d, J=20 Hz, 2H, CH2P); 6.70-7.30 (m, 9H, aromatic); 31P NMR (D2O): δ 4.4.

N-Hydroxy-N-[3-(4-methylbiphenyl)-propyl]-phosphonoacetamide (20). Compound 20 was prepared in the same manner as 9, using 4-methylphenylbenzaldehyde as starting material, as a white powder (175 mg, 48% overall yield). Anal. (C18H22NO5P.0.2 HBr) C, H, N; 1H NMR (400 MHz, D2O): δ 1.70-1.80 (m, 2H, CH2); 2.21 (s, 3H, Me), 2.46 (t, J=7.6 Hz, 2H, PhCH2); 2.66 (d, J=20 Hz, 2H, CH2P); 3.42 (t, J=7.2 Hz, 2H, CH2N); 7.0-7.30 (m, 8H, aromatic); 31P NMR (D2O): δ 15.9.

N-[3-(3-phenoxyphenyl)-propyl]-phosphonomalonamide potassium salt (21). Amine 26 (1 mmol) was coupled with malonic acid monoethyl ester according to general method C to give the ethyl ester of 21, which was then hydrolyzed with 3 equiv. of KOH in MeOH/H2O for 1 h. The reaction mixture was acidified, extracted with ethyl acetate, and the organic layer evaporated. The oily residue was dissolved in methanol, neutralized with KOH and evaporated to give 21 as a white powder (250 mg, 66% overall yield). Anal. (C18H18KNO4.0.25KCl.0.5H2O) C, H, N; 1H NMR (400 MHz, D2O): δ 1.80-1.90 (m, 2H, CH2); 2.62 (t, J=7.6 Hz, 2H, PhCH2); 3.32 (s, 2H, CH2COO); 3.33 (m, 2H, CH2N); 6.70-7.40 (m, 9H, aromatic).

A general synthetic route to the diphenylether phosphonosulfonate compounds is shown in Scheme 4.

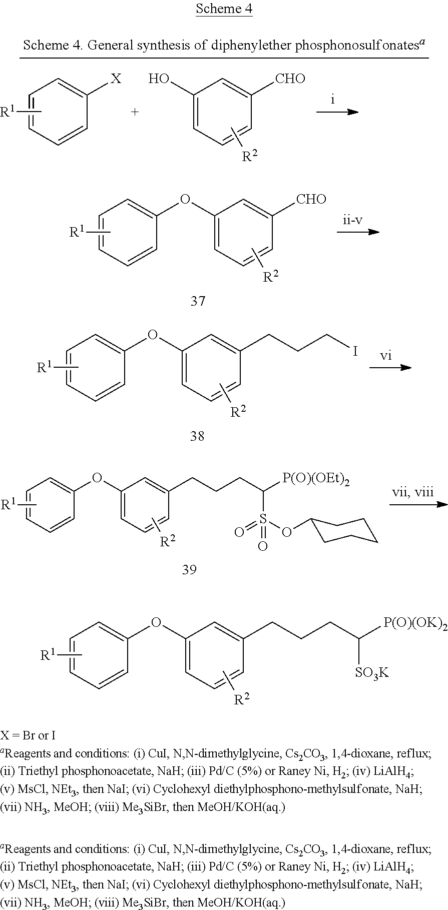

Referring to Scheme 4, if not commercially available, a 3-phenoxybenzaldehyde 37 can be prepared with a copper(I) iodide mediated coupling reaction (18) from a substituted halobenzene and a substituted hydroxybenzaldehyde, in a yield of 70-90%. The aldehyde 37 was reacted with sodium triethyl phosphonoacetate in THF to give an α,β-unsaturated carboxylate, which was hydrogenated, reduced to the alcohol by treatment with LiAlH4, mesylated, then treated with NaI to afford the iodide 38. This was then reacted with the sodium salt of cyclohexyl diethylphosphonomethylsulfonate to give a triester 39 (10), typically in an overall yield of 40% from the aldehyde 37. The triester 39 was deprotected by successive treatments with ammonia in methanol, then bromotrimethylsilane, followed by alkaline hydrolysis, affording the phosphonosulfonates as a tripotassium salt in ˜70% yield.

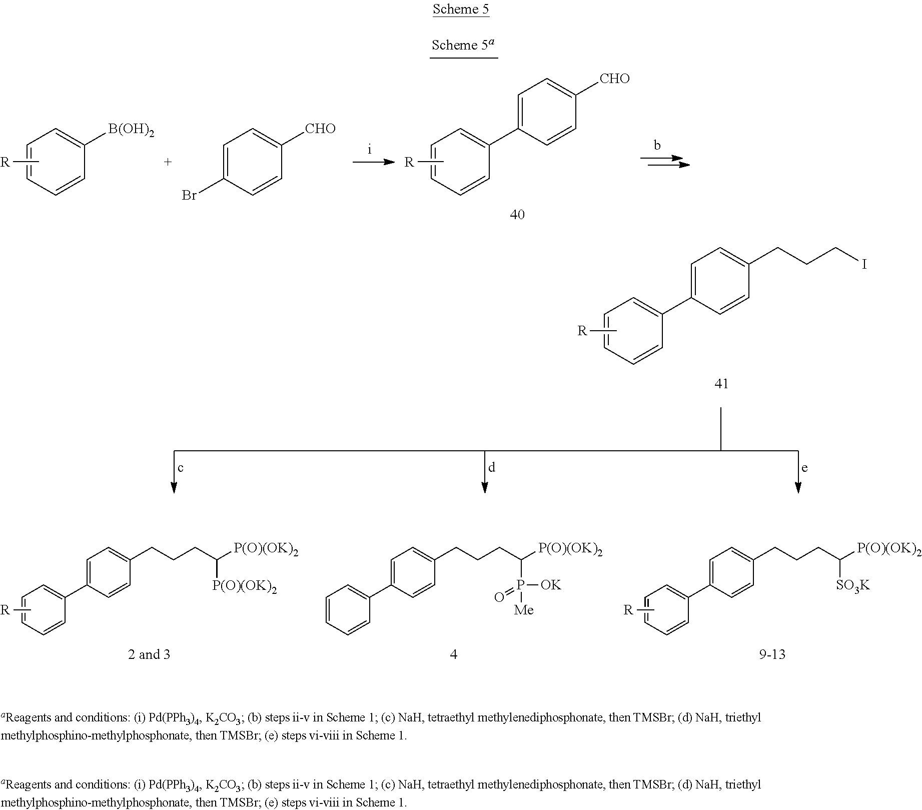

Referring to Scheme 5, biphenyl bisphosphonates and phosphonosulfonates were made similarly, as shown in Scheme 5. Iodide 41 was made from a biphenyl aldehyde 40, which is either commercially available or prepared using a Suzuki coupling reaction from 4-bromobenzaldehyde and a substituted phenylboronic acid. Compound 41 was reacted with the sodium salt of tetraethyl methylenediphosphonate or triethyl methylphosphinomethylphosphonate (19), followed by treatment with bromotrimethylsilane, to give bisphosphonates (2 and 3) or phosphinophosphonate 4, respectively. Following steps vi-viii in Scheme 4, biphenyl phosphonosulfonates (9-13) can be obtained from the iodide 41.

Compounds (S)-1 and (R)-1 were synthesized according to a published method (11), using trans-(1R,2R)—N,N′-bismethyl-cyclohexanediamine as a chiral auxiliary. The syntheses of other compounds are illustrated in Scheme 6.

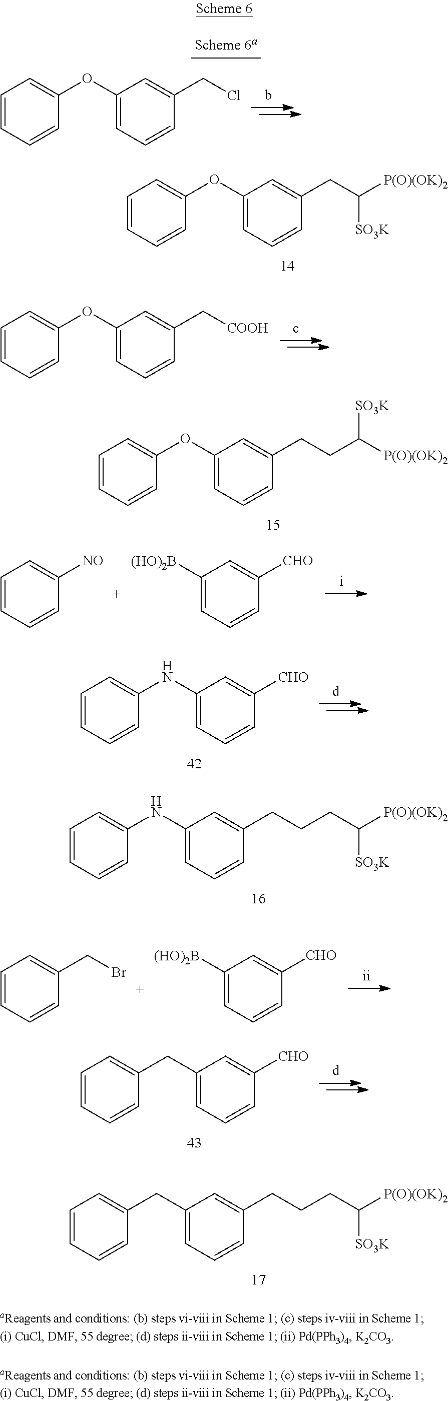

Referring to Scheme 6, compounds 14 and 15 were readily prepared from 3-phenoxybenzyl chloride and 3-phenoxyphenylacetic acid, respectively, following the general method in Scheme 4. Compound 16 was prepared from aldehyde 42, which was obtained by the coupling of nitrosobenzene and 3-formylphenylboronic acid, in the presence of copper(I) chloride (20). Suzuki coupling of benzyl bromide and 3-formylphenylboronic acid afforded 3-phenylaminobenzaldehyde 43. Compound 17 was then made from aldehyde 43, following steps ii-viii in Scheme 4. Similarly, Compounds 18 and 19 were made following steps ii-viii in Scheme 4, starting from available 4-phenoxybenzaldehyde and 9-ethyl-3-carbazole-carboxaldehyde, respectively.

### Experimental Section

All reagents used were purchased from Aldrich (Milwaukee, Wis.). The purities of all compounds were routinely monitored by using 1H and 31P NMR spectroscopy at 400 or 500 MHz on Varian (Palo Alto, Calif.) Unity spectrometers. Elemental analysis results are provided in Table S2.

TABLE S2
Elemental analysis results of compounds.

| Compound | | required C | required H | required N | found C | found H | found N |
|---|---|---|---|---|---|---|---|
| 1 | (FX24B-04-652) | 30.57 | 2.73 | | 30.5 | 2.53 | |
| (R)-1 | (FX42-R1) | 22.63 | 1.9 | | 22.49 | 2.16 | |
| (S)-1 | (FX24A-02) | 26.08 | 2.6 | | 26.07 | 2.56 | |
| 3 | (FX43-3) | 38.35 | 3.6 | | 38.357 | 3.55 | |
| 4 | (FX44-4) | 27.31 | 2.97 | | 27.14 | 3.26 | |
| 5 | (FX23-01) | 35.22 | 3.33 | | 35.45 | 3.39 | |
| 6 | (FX29-08-701) | 27.55 | 2.68 | | 27.79 | 2.42 | |
| 7 | (FX34-13) | 34.28 | 3.05 | | 34.28 | 3.21 | |
| 8 | (FX45-8) | 41.68 | 4.56 | | 41.7 | 4.23 | |
| 9 | (FX46-9) | 24.57 | 2.06 | | 24.8 | 1.98 | |
| 10 | (FX47-10) | 35.44 | 3.33 | | 35.4 | 3.49 | |
| 11 | (FX48-11) | 22.31 | 1.98 | | 22.07 | 1.92 | |
| 12 | (FX49-12) | 27.49 | 2.77 | | 27.23 | 2.71 | |
| 13 | (FX50-13) | 38.07 | 4.2 | | 38.2 | 4.14 | |
| 14 | (FX51-14) | 37.05 | 3.5 | | 36.9 | 3.5 | |
| 15 | (FX52-15) | 36.71 | 3.28 | | 36.96 | 3.52 | |
| 16 | (FX53-16) | 29.76 | 3.12 | 2.17 | 29.99 | 3.1 | 2.24 |
| 17 | (FX54-17) | 37.37 | 3.69 | | 37.16 | 3.82 | |
| 18 | (FX55-18) | 38.39 | 3.22 | | 38.12 | 3.56 | |
| 19 | (FX56-19) | 40.92 | 4.16 | 2.51 | 41.17 | 4.36 | 2.85 |
| 20 | (FX35-14) | 28.15 | 2.95 | | 28.05 | 2.63 | |
| 21 | (FX28-07) | 34.09 | 3.58 | | 34.2 | 3.36 | |
| 22 | (FX38-17) | 35.91 | 2.66 | | 35.87 | 2.95 | |
| 24 | (FX33-12) | 37.2 | 3.64 | | 37.281 | 3.669 | |
| 25 | (FX40-19-760) | 45.67 | 5.14 | | 45.63 | 4.93 | |
| 26 | (FX26-05-769) | 45.38 | 3.97 | | 45.55 | 4.06 | |
| 27 | (FX57-27) | 18.36 | 2.12 | | 18.3 | 1.91 | |
| 28 | (FX58-28) | 38.9 | 3.41 | | 38.82 | 3.38 | |
| 29 | (FX59-29) | 28.88 | 2.54 | | 29.08 | 2.74 | |
| 30 | (FX25-03) | 33.77 | 3.67 | | 33.96 | 3.71 | |
| 31 | (FX60-31) | 31.58 | 3.59 | | 31.686 | 3.415 | |
| 32 | (FX61-32) | 37.49 | 3.5 | | 37.5 | 3.27 | |
| 33 | (FX32-11) | 34.65 | 2.91 | | 34.79 | 2.89 | |
| 34 | (FX62-34) | 29.45 | 2.74 | | 29.85 | 2.91 | |
| 35 | (FX27-06) | 32.88 | 2.76 | | 32.89 | 2.85 | |
| 36 | (FX63-36) | 32.29 | 3.35 | | 32.38 | 3.51 | |

General Method F (steps ii-viii in Scheme 4). Step ii: Triethyl phosphonoacetate (3.3 mmol) was added dropwise to NaH (145 mg, 60% in oil, 3.6 mmol) suspended in dry THF (7 mL) at 0° C. To the resulting clear solution was added a benzaldehyde (3 mmol) and, after stirring at room temperature for 0.5 h, the reaction mixture was partitioned between diethyl ether (50 mL) and water (50 mL). The organic layer was dried and evaporated. Step iii: The residue oil was then hydrogenated in MeOH (15 mL), in the presence of 5% Pd/C (50 mg), or Raney Ni (500 mg) when the aldehyde 37 was prepared using the CuI mediated reaction (Scheme 4). The catalyst was filtered and the filtrate was concentrated and dried in vacuo. Step iv: The resulting oil was dissolved in anhydrous THF (8 mL) and LiAlH₄ (114 mg) slowly added to the solution at 0° C. After 1 h, the reaction was carefully quenched by adding a few drops of water, and the reaction mixture filtered. Step v: The filtrate was evaporated to dryness and the alcohol thus obtained redissolved in CH₂Cl₂ (10 mL) containing NEt3 (0.5 mL, 3.6 mmol). Methanesulfonyl chloride (230 μL, 3 mmol) was added slowly at 0° C. After 1 h stirring at room temperature, diethyl ether (50 mL) and water (50 mL) was added and the organic layer was collected, washed with 1 N HCl and saturated NaHCO3, dried, and evaporated to dryness. The oily residue was treated with NaI (1.35 g, 9 mmol) in acetone (7 mL) at 60° C. for 1 h. The reaction mixture was then partitioned between diethyl ether (50 mL) and water (50 mL) and the organic layer washed with 5% Na₂S₂O₃, dried and evaporated to dryness to give an iodide, such as 38. The iodide thus obtained was quite pure, according to ¹H and ¹³C NMR spectra, and may be used in the next step without purification. Step vi: Cyclohexyl diethylphosphonomethylsulfonate (470 mg, 1.5 mmol) was added to NaH (60 mg, 60% in oil, 1.5 mmol) suspended in dry DMF (2 mL) at 0° C. To the resulting clear solution was added an iodide (1 mmol) and, after stirring at room temperature for 3 h, the reaction mixture was partitioned between diethyl ether (50 mL) and water (50 mL). The organic layer was dried and evaporated and the residue subjected to a column chromatography (silica gel, ethyl acetate:hexane/1:1) to give a phosphonosulfonate triester, such as 39, as a colorless oil. Step vii: The triester was treated with ammonium hydroxide (12M, 1 mL) in MeOH (6 mL) at 60° C. for 3 h. The solvents were evaporated and the residue subjected to ion exchange chromatography (DOWEX® 50WX8-200, H+ form, 3 mL) using MeOH as eluent. Step viii: The eluent was evaporated to dryness and the resulting diethylphosphonosulfonic acid dissolved in anhydrous CH₃CN (3 mL) and treated with Me₃SiBr (400 μL, 3 mmol) at 40° C., overnight. The solution was evaporated to dryness and MeOH (5 mL) added to the residue. The solvent was removed in vacuo again, and the residue redissolved in MeOH (5 mL). Neutralization with 2 M KOH to pH=8 gave a tripotassium salt of a phosphonosulfonate as a white powder.

General Method G (step i in Scheme 4) (18). A mixture containing a halobenzene (3 mmol), a 3-hydroxybenzaldehyde (4.5 mmol), CuI (58 mg, 0.3 mmol), N,Ndimethylglycine (93 mg, 0.9 mmol) and Cs₂CO₃ (2 g, 6 mmol) in 1,4-dioxane (8 mL) was vigorously stirred at 90° C. for 18 h. The solvent was evaporated and the residue partitioned between diethyl ether (50 mL) and water (50 mL). The organic layer was successively washed with 5% NaOH (2×20 mL), water (20 mL) and saturated NaCl (20 mL). It was then dried and evaporated to give aldehyde 37 as a pale yellow oil, which is quite pure and may be used in the next step directly. It may also be purified via a column chromatography.

1-Phosphono-4-(3-phenoxyphenyl)butylsulfonic acid tripotassium salt, 1. Compound 1 was prepared from 3-phenoxybenzaldehyde (3 mmol), following general method F as a white powder (680 mg, 36% overall yield). Anal. (C₁₆H₁₆K₃O₇PS.KBr.0.5H₂O) C, H; ¹H NMR (400 MHz, D2O): δ 1.50-1.90 (m, 4H, —CH₂CH₂—); 2.40-2.50 (m, 2H, PhCH₂), 2.70-2.80 (m, 1H, CHSO₃K), 6.70-7.30 (m, 9H, aromatic); ³¹P NMR (D₂O): δ 12.4.

4-(4-Biphenyl)butyldiphosphonic acid tetrapotassium salt, 2. Compound 2 was prepared from 4-phenylbenzaldehyde (3 mmol), following steps ii-vi, then step viii of the general method F as a white powder (721 mg, 46% overall yield). Purity was determined to be 87.3% by quantitative NMR spectroscopy. ¹H NMR (400 MHz, D₂O): δ 1.60-1.80 (m, 5H, —CH₂CH₂— and CH); 2.56 (t, J=5.6 Hz, 2H, PhCH₂), 7.20-7.60 (m, 9H, aromatic); ₃₁P NMR (D₂O): δ 21.2.

4-[4-(4-Trifluoromethylphenyl)phenyl)]butyldiphosphonic acid dipotassium salt, 3. Compound 3 was prepared from 4-trifluoromethylphenylbenzaldehyde (3 mmol), following steps ii-vi, then step viii of general method F, as a white powder (671 mg, 42% overall yield). Anal. (C₁₇H₁₇F₃K₂O₆P₂) C, H; ¹H NMR (400 MHz, D₂O): δ 1.60-1.90 (m, 5H, —CH₂CH₂— and CH); 2.58 (t, J=5.6 Hz, 2H, PhCH₂), 7.20-7.60 (m, 9H, aromatic); ³¹P NMR (D₂O): δ 21.5.

4-(4-Biphenyl)butyldiphosphonic acid dipotassium salt, 4. Compound 4 was prepared from 4-phenylbenzaldehyde (3 mmol), following steps ii-vi, then step viii of general method F, as a white powder (470 mg, 21% overall yield). Anal. (C₁₇H₂₀K₂O₅P₂) C, H; ^{1H} NMR (400 MHz, D₂O): δ 1.19 (d, J=11.2 Hz, 3H, Me), 1.60-1.80 (m, 5H, —CH₂CH₂— and CH); 2.56 (t, J=5.6 Hz, 2H, PhCH₂), 7.20-7.60 (m, 9H, aromatic); ³¹P NMR (D₂O): δ 18.3 (s, 1P), 49.6 (s, 1P).

1-Phosphono-4-[3-(4-fluorophenoxy)phenyl]butylsulfonic acid tripotassium salt, 5. Compound 5 was prepared from 4-fluoro-iodobenzene (3 mmol) and 3-hydroxybenzaldehyde (4.5 mmol), following general methods G and F, as a white powder (425 mg, 26% overall yield). Anal. (C₁₆H₁₅FK₃O₇PS.1.5H₂O) C, H; ¹H NMR (400 MHz, D₂O): δ 1.60-1.90 (m, 4H, —CH₂CH₂—); 2.40-2.50 (m, 2H, PhCH₂), 2.80-2.90 (m, 1H, CHSO₃K), 6.60-7.10 (m, 8H, aromatic); ³¹P NMR (D2O): δ 13.6.

1-Phosphono-4-[3-(4-propylphenoxy)phenyl]butylsulfonic acid tripotassium salt, 6. Compound 6 was prepared from 4-propyl-bromobenzene (3 mmol) and 3-hydroxybenzaldehyde (4.5 mmol), following general methods B and A, as a white powder (670 mg, 27% overall yield). Anal. (C₁₉H₂₂K₃O₇PS.2.4KBr) C, H; ¹H NMR (400 MHz, D₂O): δ 0.73 (t, J=7.2 Hz, 3H, CH₃), 1.40-1.50 (m, 2H, CH₃CH₂), 1.60-1.90 (m, 4H, —CH₂CH₂—); 2.40-2.50 (m, 4H, PhCH₂ and PhCH₂), 2.80-2.90 (m, 1H, CHSO₃K), 6.70-7.20 (m, 8H, aromatic); ³¹P NMR (D₂O): δ 14.4.

1-Phosphono-4-[3-(4-trifluoromethylphenoxy)phenyl]butylsulfonic acid tripotassium salt, 7. Compound 7 was prepared from 4-trifluoromethyl-iodobenzene (3 mmol) and 3-hydroxybenzaldehyde (4.5 mmol), following general methods G and F, as a white powder (570 mg, 32% overall yield). Anal. (C₁₇H₁₅F₃K₃O₇PS.1.5H₂O) C, H; ¹H NMR (400 MHz, D₂O): δ 1.80-2.00 (m, 4H, —CH₂CH₂—); 2.40-2.60 (m, 2H, PhCH₂), 2.80-2.90 (m, 1H, CHSO₃K), 6.80-7.40 (m, 8H, aromatic); ³¹P NMR (D₂O): δ 13.9.

1-Phosphono-4-[3-(2-benzylphenoxy)phenyl]butylsulfonic acid tripotassium salt, 8. Compound 8 was prepared from 2-benzyl-iodobenzene (3 mmol) and 3-hydroxybenzaldehyde (4.5 mmol), following general methods G and then F, as a white powder (570 mg, 29% overall yield). Anal. (C₂₃H₂₂K₃O₇PS.4H₂O) C, H; ¹H NMR (400 MHz, D₂O): δ 1.60-1.90 (m, 4H, —CH₂CH₂—); 2.40-2.50 (m, 2H, PhCH₂), 2.80-2.90 (m, 1H, CHSO₃K), 3.80 (s, 2H, PhCH₂Ph), 6.50-7.30 (m, 13H, aromatic); ³¹P NMR (D₂O): δ 12.7.

1-Phosphono-4-(4-biphenyl)butylsulfonic acid tripotassium salt, 9. Compound 9 was prepared from 4-phenylbenzaldehyde (3 mmol), following general method F, as a white powder (730 mg, 31% overall yield). Anal. (C₁₆H₁₆K₃O₆PS.2.5KBr) C, H; ^{1H} NMR (400 MHz, D₂O): δ 1.70-1.90 (m, 4H, —CH₂CH₂—); 2.50-2.60 (m, 2H, PhCH₂), 2.90-3.00 (m, 1H, CHSO₃K), 7.20-7.60 (m, 9H, aromatic); ³¹P NMR (D₂O): δ 14.4.

1-Phosphono-4-[4-(2,4-difluorophenyl)phenyl]butylsulfonic acid tripotassium salt, 10. 4-(2,4-difluorophenyl)benzaldehyde was made by a Suzuki coupling reaction from 2,4-difluorophenylboronic acid (3.6 mmol) and 4-bromobenzaldehyde (3 mmol), as described previously (21). Compound 10 was prepared from the aldehyde thus obtained, following general method F, as a white powder (470 mg, 28% overall yield). Anal. (C₁₆H₁₄F₂K₃O₆PS) C, H; ¹H NMR (400 MHz, D₂O): δ 1.65-2.00 (m, 4H, —CH₂CH₂—); 2.45-2.60 (m, 2H, PhCH₂), 2.80-2.85 (m, 1H, CHSO₃K), 6.80-7.40 (m, 7H, aromatic); ³¹P NMR (D₂O): δ 12.5.

1-Phosphono-4-[4-(4-methylphenyl)phenyl]butylsulfonic acid tripotassium salt (11). Compound 11 was prepared from 4-(4-methylphenyl)benzaldehyde (3 mmol), following general method F, as a white powder (770 mg, 28% overall yield). Anal. (C₁₇H₁₈K₃O₆PS.3.5KBr) C, H; ¹H NMR (400 MHz, D₂O): δ 1.70-1.90 (m, 4H, —CH₂CH₂—); 2.21 (s, 3H, Me), 2.40-2.50 (m, 2H, PhCH₂), 2.90-3.00 (m, 1H, CHSO₃K), 7.20-7.50 (m, 8H, aromatic); ³¹P NMR (D₂O): δ 14.5.

1-Phosphono-4-[4-(4-butylphenyl)phenyl]butylsulfonic acid tripotassium salt 12. 4-(4-Butylphenyl)benzaldehyde was made by a Suzuki coupling reaction from 4-butylphenylboronic acid (3.6 mmol) and 4-bromobenzaldehyde (3 mmol) (21). Compound 12 was prepared from the aldehyde thus obtained, following general method F, as a white powder (650 mg, 25% overall yield). Anal. (C₂₀H₂₄K₃O₆PS.2.8 KBr) C, H; ¹H NMR (400 MHz, D₂O): δ 0.74 (t, J=7.2 Hz, 3H, CH₃), 1.10-1.20 (m, 2H, CH₃CH₂), 1.40-1.50 (m, 2H, CH₃CH₂CH₂), 1.650-2.00 (m, 4H, —CH₂CH₂—); 2.45-2.60 (m, 4H, PhCH₂ and PhCH₂), 2.70-2.80 (m, 1H, CHSO₃K), 7.20-7.50 (m, 8H, aromatic); ³¹P NMR (D₂O): δ 12.4.

1-Phosphono-4-[4-(3-propoxyphenyl)phenyl]butylsulfonic acid tripotassium salt 13. 4-(3-propoxyphenyl)-benzaldehyde was made by a Suzuki coupling reaction from 3-propoxyphenylboronic acid (3.6 mmol) and 4-bromobenzaldehyde (3 mmol)²¹. Compound 13 was prepared from the aldehyde thus obtained, following general method F, as a white powder (539 mg, 30% overall yield). Anal. (C₁₉H₂₂K₃O₆PS) C, H; ¹H NMR (400 MHz, D₂O): δ 0.74 (t, J=7.2 Hz, 3H, CH₃), 1.40-1.50 (m, 2H, CH₃CH₂), 1.65-2.00 (m, 4H, —CH₂CH₂), 2.45-2.60 (m, 2H, PhCH₂), 2.80-2.85 (m, 1H, CHSO₃K), 3.75 (t, J=7.2 Hz, 2H, OCH₂), 6.80-7.40 (m, 7H, aromatic); ³¹P NMR (D₂O): δ 12.8.

(1S)-1-Phosphono-4-(3-phenoxyphenyl)butylsulfonic acid tripotassium salt (S)-1. Compound (S)-1 was prepared from methylphosphonic dichloride (3 mmol) and trans-(1R,2R)—N,N′-bismethyl-cyclohexanediamine (3 mmol), following a published method (11), as a white powder (640 mg, 29% overall yield). Anal. (C₁₆H₁₆K₃O₇PS. 1.2K₂SO₄.1.5.H₂O) C, H; Identical NMR spectra as 1.

(1R)-1-Phosphono-4-(3-phenoxyphenyl)butylsulfonic acid tripotassium salt (R)-1. Compound (R)-1 was obtained as a minor product during the synthesis of (S)-1, as described immediately above (405 mg, 16% overall yield). Anal. (C₁₆H₁₆K₃O₇PS.2K₂SO₄) C, H; Identical NMR spectra as 1.

1-Phosphono-2-(3-phenoxyphenyl)ethylsulfonic acid tripotassium salt 14. Compound 14 was prepared from 3-phenoxybenzyl chloride (1 mmol), following steps vi-viii of general method F, as a white powder (285 mg, 55% overall yield). Anal. (C₁₄H₁₂K₃O₇PS.C₂H₅OH) C, H; ¹H NMR (400 MHz, D₂O): δ 2.95-3.05 (m, 1H, CH₂); 3.10-3.30 (m, 2H, CH and CH₂), 6.70-7.30 (m, 9H, aromatic); ³¹P NMR (D2O): δ 13.8.

1-Phosphono-3-(3-phenoxyphenyl)propylsulfonic acid tripotassium salt 15. Compound 15 was prepared from 3-phenoxyphenylacetic acid (3 mmol), following steps iv-viii of general method F, as a white powder (380 mg, 25% overall yield). Anal. (C₁₅H₁₄K₃O₇PS.0.25C₂H₅OH.0.5H₂O) C, H; ¹H NMR (400 MHz, D₂O): δ δ 2.00-2.10 (m, 2H, CH₂); 2.65-2.80 (m, 2H, PhCH₂), 2.90-3.00 (m, 1H, CHSO₃K), 6.70-7.30 (m, 9H, aromatic); ³¹P NMR (D₂O): δ 14.1.

1-Phosphono-4-(3-phenylaminophenyl)butylsulfonic acid tripotassium salt 16. A mixture of nitrosobenzene (3 mmol) and CuCl (3 mmol) in anhydrous DMF (8 mL) was heated to 55° C. for 0.5 h. Then, 3-formylphenylboronic acid (3.3 mmol) was added to the reaction mixture, which was then stirred for another 16 h²⁰. The product was then partitioned between diethyl ether (50 mL) and water (50 mL) and the organic layer washed with saturated NaHCO₃, dried, then evaporated to dryness, giving 3-phenylaminobenzaldehyde as a pale yellow oil, which may be used directly without purification. Compound 16 was prepared from the aldehyde thus obtained, following general method F, as a white powder (425 mg, 22% overall yield). Anal. (C₁₆H₁₇NK₃O₇PS.KBr.1.5H₂O) C, H, N; ¹H NMR (400 MHz, D₂O): δ 1.60-1.90 (m, 4H, —CH₂CH₂—); 2.40-2.50 (m, 2H, PhCH₂), 2.80-2.90 (m, 1H, CHSO₃K), 6.70-7.20 (m, 9H, aromatic); ³¹P NMR (D₂O): δ 14.4.

1-Phosphono-4-(3-benzylphenyl)butylsulfonic acid tripotassium salt 17. 3-Benzylbenzaldehyde was prepared from benzyl bromide (3 mmol) and 3-formylphenylboronic acid (3.3 mmol) by a Suzuki coupling reaction²¹. Compound 17 was prepared from the aldehyde thus obtained, following general method F, as a white powder (540 mg, 33% overall yield). Anal. (C₁₇H₂₀K₃O₇PS.0.25KBr.H₂O) C, H; ¹H NMR (400 MHz, D₂O): δ 1.50-1.90 (m, 4H, —CH₂CH₂—); 2.40-2.50 (m, 2H, PhCH₂), 2.80-2.90 (m, 1H, CHSO₃K), 3.88 (s, 2H, PhCH₂Ph), 6.90-7.20 (m, 9H, aromatic); ³¹P NMR (D₂O): δ 13.8.

1-Phosphono-4-(4-phenoxyphenyl)butylsulfonic acid tripotassium salt 18. Compound 18 was prepared from 4-phenoxybenzaldehyde (3 mmol), following general method F, as a white powder (680 mg, 36% overall yield). Anal. (C₁₆H₁₆K₃O₇PS) C, H; ¹H NMR (400 MHz, D₂O): δ 1.60-1.90 (m, 4H, —CH₂CH₂); 2.40-2.50 (m, 2H, PhCH₂), 2.80-2.90 (m, 1H, CHSO₃K), 6.80-7.30 (m, 9H, aromatic); ³¹P NMR (D₂O): δ 13.7.

1-Phosphono-4-(9-ethylcarbazole-3-yl)butylsulfonic acid tripotassium salt 19. Compound 19 was prepared from 9-ethyl-3-carbazole-carboxaldehyde (3 mmol), following general method F, as a white powder (600 mg, 36% overall yield). Anal. (C₁₈H₁₉NK₃O₆PS.0.5C₂H₅OH.0.5H₂O) C, H, N; ¹H NMR (400 MHz, D₂O): δ 1.14 (t, J=7.2 Hz, 3H, CH₃), 1.70-1.90 (m, 4H, —CH₂CH₂—); 2.60-2.70 (m, 2H, PhCH₂), 2.80-2.90 (m, 1H, CHSO₃K), 4.22 (q, J=7.2 Hz, 3H, NCH₂), 7.05-7.40 (m, 5H, aromatic), 7.92 (s, 1H, aromatic), 8.00 (d, J=8.0 Hz, 1H, aromatic); ³¹P NMR (D₂O): δ 14.1

1-Phosphono-4-[3-(3-fluorophenoxy)phenyl]butylsulfonic acid tripotassium salt 20. Compound 20 was prepared from 3-fluoro-iodobenzene (3 mmol) and 3-hydroxybenzaldehyde (4.5 mmol), following general methods G and F, as a white powder (570 mg, 28% overall yield). Anal. (C₁₆H₁₅FK₃O₇PS.KBr.2.5H₂O) C, H; ¹H NMR (400 MHz, D₂O): δ 1.60-1.90 (m, 4H, —CH₂CH₂—); 2.50-2.70 (m, 2H, PhCH₂), 2.80-2.90 (m, 1H, CHSO₃K), 6.70-7.20 (m, 8H, aromatic); ³¹P NMR (D₂O): δ 13.8.

1-Phosphono-4-[3-(2-fluorophenoxy)phenyl]butylsulfonic acid tripotassium salt 21. Compound 21 was prepared from 2-fluoro-iodobenzene (3 mmol) and 3-hydroxybenzaldehyde (4.5 mmol), following general methods G and F, as a white powder (340 mg, 20% overall yield). Anal. (C₁₆H₁₅FK₃O₇PS.2.5H₂O) C, H; ¹H NMR (400 MHz, D₂O): δ 1.70-1.90 (m, 4H, —CH₂CH₂—); 2.60-2.80 (m, 2H, PhCH₂), 2.90-3.00 (m, 1H, CHSO₃K), 6.60-7.20 (m, 8H, aromatic); ³¹P NMR (D₂O): δ 13.2.

1-Phosphono-4-[3-(3-trifluoromethylphenoxy)phenyl]butylsulfonic acid tripotassium salt 22. Compound 22 was prepared from 3-(3-trifluoromethylphenoxy)benzaldehyde (3 mmol), following general method F, as a white powder (600 mg, 30% overall yield). Anal. (C₁₇H₁₅F₃K₃O₇PS) C, H; ¹H NMR (400 MHz, D₂O): δ 1.80-2.00 (m, 4H, —CH₂CH₂—); 2.50-2.70 (m, 2H, PhCH₂), 2.85-2.95 (m, 1H, CHSO₃K), 6.80-7.40 (m, 8H, aromatic); ³¹P NMR (D₂O): δ 13.3.

1-Phosphono-4-[3-(2-trifluoromethylphenoxy)phenyl]butylsulfonic acid tripotassium salt 23. Compound 23 was prepared from 2-trifluoromethyliodobenzene (3 mmol) and 3-hydroxybenzaldehyde (4.5 mmol), following general methods G and F, as a white powder (205 mg, 12% overall yield). Purity was determined to be 86.5% by quantitative NMR spectroscopy. ¹H NMR (400 MHz, D₂O): δ 1.70-1.90 (m, 4H, —CH₂CH₂—); 2.50-2.65 (m, 2H, PhCH₂), 2.80-2.90 (m, 1H, CHSO₃K), 6.70-7.30 (m, 8H, aromatic); ³¹P NMR (D₂O): δ 13.7.

1-Phosphono-4-[3-(4-chlorophenoxy)phenyl]butylsulfonic acid tripotassium salt 24. Compound 24 was prepared from 3-(4-chlorophenoxy)benzaldehyde (3 mmol), following general method F, as a white powder (525 mg, 30% overall yield). Anal. (C₁₆H₁₅ClK₃O₇PS.C₂H₅OH) C, H; ¹H NMR (400 MHz, D₂O): δ 1.60-1.90 (m, 4H, —CH₂CH₂—); 2.45-2.55 (m, 2H, PhCH₂), 2.80-2.90 (m, 1H, CHSO₃K), 6.70-7.10 (m, 8H, aromatic); ³¹P NMR (D₂O): δ 14.0.

1-Phosphono-4-[3-(4-tert-butylphenoxy)phenyl]butylsulfonic acid tripotassium salt 25. Compound 25 was prepared from 3-(4-tert-butylphenoxy)benzaldehyde (3 mmol), following general method F, as a white powder (610 mg, 35% overall yield). Anal. (C₂₀H₂₄K₃O₇PS.1.5H₂O) C, H; ¹H NMR (400 MHz, D₂O): δ 1.10 (s, 9H, CMe₃), 1.60-1.85 (m, 4H, —CH₂CH₂—); 2.40-2.50 (m, 2H, PhCH₂), 2.80-2.90 (m, 1H, CHSO₃K), 6.60-7.40 (m, 8H, aromatic); ³¹P NMR (D₂O): δ 14.3.

1-Phosphono-4-[3-(4-benzylphenoxy)phenyl]butylsulfonic acid tripotassium salt 26. Compound 26 was prepared from 4-benzyl-iodobenzene (3 mmol) and 3-hydroxybenzaldehyde (4.5 mmol), following general methods G and F, as a white powder (510 mg, 28% overall yield). Anal. (C₂₃H₂₂K₃O₇PS.H₂O) C, H; ¹H NMR (400 MHz, D₂O): δ 1.60-1.90 (m, 4H, —CH₂CH₂—); 2.40-2.50 (m, 2H, PhCH₂), 2.75-2.85 (m, 1H, CHSO₃K), 3.79 (s, 2H, PhCH₂), 6.60-7.20 (m, 13H, aromatic); ³¹P NMR (D₂O): δ 13.9.

1-Phosphono-4-[3-(4-hydroxyphenoxy)phenyl]butylsulfonic acid tripotassium salt 27. Compound 27 was prepared from 4-tert-butoxy-iodobenzene (3 mmol) and 3-hydroxybenzaldehyde (4.5 mmol), following general methods G and F, as a white powder (690 mg, 22% overall yield). Anal. (C₁₆H₁₆K₃O₈PS.4KBr.3H₂O) C, H; ¹H NMR (400 MHz, D₂O): δ 1.60-1.90 (m, 4H, —CH₂CH₂—); 2.40-2.50 (m, 2H, PhCH₂), 2.90-3.00 (m, 1H, CHSO₃K), 6.60-7.20 (m, 8H, aromatic); ³¹P NMR (D₂O): δ 14.5.

1-Phosphono-4-[3-(4-phenoxyphenoxy)phenyl]butylsulfonic acid tripotassium salt 28. Compound 28 was prepared from 4-phenoxy-iodobenzene (3 mmol) and 3-hydroxybenzaldehyde (4.5 mmol), following general methods G and F, as a white powder (610 mg, 30% overall yield). Anal. (C₂₂H₂₀K₃O₈PS.0.5KBr.1.5H₂O) C, H; ¹H NMR (400 MHz, D₂O): δ 1.60-1.90 (m, 4H, —CH₂CH₂—); 2.40-2.50 (m, 2H, PhCH₂), 2.80-2.90 (m, 1H, CHSO₃K), 6.60-7.25 (m, 13H, aromatic); ³¹P NMR (D₂O): δ 13.7.

1-Phosphono-4-[3-[4-(furan-2-yl)phenoxy]phenyl]butylsulfonic acid tripotassium salt 29. Compound 29 was prepared from 4-(furan-2-yl)-iodobenzene (3 mmol) and 3-hydroxybenzaldehyde (4.5 mmol), following general methods G and F, as a white powder (690 mg, 22% overall yield). Anal. (C₂₀H₁₈K₃O₈PS.2KBr.1.5H₂O) C, H; ¹H NMR (400 MHz, D₂O): δ 1.60-1.90 (m, 4H, —CH₂CH₂—); 2.40-2.50 (m, 2H, PhCH₂), 2.70-2.80 (m, 1H, CHSO₃K), 6.40-7.60 (m, 11H, aromatic); ³¹P NMR (D₂O): δ 12.4.

1-Phosphono-4-[3-(3,4-difluorophenoxy)phenyl]butylsulfonic acid tripotassium salt 30. Compound 30 was prepared from 3,4-difluoro-iodobenzene (3 mmol) and 3-hydroxybenzaldehyde (4.5 mmol), following general methods G and F, as a white powder (525 mg, 29% overall yield). Anal. (C₁₆H₁₄F₂K₃O₇PS.0.5C₂H₅OH.2.5H₂O) C, H; ¹H NMR (400 MHz, D₂O): δ 1.60-2.00 (m, 4H, —CH₂CH₂—); 2.40-2.50 (m, 2H, PhCH₂), 2.80-2.90 (m, 1H, CHSO₃K), 6.50-7.30 (m, 7H, aromatic); ³¹P NMR (D₂O): δ 12.8.

1-Phosphono-4-[3-(3,4-dichlorophenoxy)phenyl]butylsulfonic acid tripotassium salt 31. Compound 31 was prepared from 3-(3,4-dichlorophenoxy)benzaldehyde (3 mmol), following general method F, as a white powder (540 mg, 28% overall yield). Anal. (C₁₆H₁₄Cl₂K₃O₇PS.0.5C₂H₅OH.3H₂O) C, H; ¹H NMR (400 MHz, D₂O): δ 1.60-2.00 (m, 4H, —CH₂CH₂—); 2.40-2.50 (m, 2H, PhCH₂), 2.80-2.90 (m, 1H, CHSO₃K), 6.80-7.40 (m, 7H, aromatic); ³¹P NMR (D₂O): δ 12.4.

1-Phosphono-4-[3-(benzofuran-5-yloxy)phenyl]butylsulfonic acid tripotassium salt 32. Compound 32 was prepared from 5-bromobenzofuran (3 mmol) and 3-hydroxybenzaldehyde (4.5 mmol), following general methods G and F, as a white powder (380 mg, 22% overall yield). Anal. (C₁₈H₁₆K₃O₈PS.2H₂O) C, H; ¹H NMR (400 MHz, D₂O): δ 1.60-1.90 (m, 4H, —CH₂CH₂—); 2.40-2.50 (m, 2H, PhCH₂), 2.80-2.90 (m, 1H, CHSO₃K), 6.60-7.60 (m, 9H, aromatic); ³¹P NMR (D₂O): δ 14.1.

1-Phosphono-4-[3-(3,5-difluorophenoxy)phenyl]butylsulfonic acid tripotassium salt 33. Compound 33 was prepared from 3,5-difluoro-iodobenzene (3 mmol) and 3-hydroxybenzaldehyde (4.5 mmol), following general methods G and F, as a white powder (415 mg, 25% overall yield). Anal. (C₁₆H₁₄F₂K₃O₇PS.H₂O) C, H; ¹H NMR (400 MHz, D₂O): δ 1.60-1.90 (m, 4H, —CH₂CH₂—); 2.40-2.50 (m, 2H, PhCH₂), 2.80-2.90 (m, 1H, CHSO₃K), 6.40-7.20 (m, 7H, aromatic); ³¹P NMR (D₂O): δ 14.2.

1-Phosphono-4-[3-(3,5-dichlorophenoxy)phenyl]butylsulfonic acid tripotassium salt 34. Compound 34 was prepared from 3-(3,5-dichlorophenoxy)benzaldehyde (3 mmol), following general method F, as a white powder (570 mg, 26% overall yield). Anal. (C₁₆H₁₄Cl₂K₃O₇PS.C₂H₅OH.KBr) C, H; ¹H NMR (400 MHz, D₂O): δ 1.60-1.90 (m, 4H, —CH₂CH₂—); 2.45-2.55 (m, 2H, PhCH₂), 2.85-2.95 (m, 1H, CHSO₃K), 6.80-7.40 (m, 7H, aromatic); ³¹P NMR (D₂O): δ 12.8.

1-Phosphono-4-[3-(4-fluorophenoxy)-6-fluoro-phenyl]butylsulfonic acid tripotassium salt 35. Compound 35 was prepared from 4-fluoro-phenol (4.5 mmol) and 3-bromo-6-fluorobenzaldehyde (3 mmol), following general methods G and F, as a white powder (525 mg, 30% overall yield). Anal. (C₁₆H₁₄F₂K₃O₇PS.0.25KBr.H₂O) C, H; ¹H NMR (400 MHz, D₂O): δ 1.60-1.90 (m, 4H, —CH₂CH₂—); 2.50-2.70 (m, 2H, PhCH₂), 2.75-2.85 (m, 1H, CHSO₃K), 6.60-7.00 (m, 7H, aromatic); ³¹P NMR (D₂O): δ 13.6.

1-Phosphono-4-[3-(4-fluorophenoxy)-6-methoxy-phenyl]butylsulfonic acid tripotassium salt 36. Compound 36 was prepared from 4-fluorophenol (4.5 mmol) and 3-bromo-6-methoxybenzaldehyde (3 mmol), following general methods G and F, as a white powder (470 mg, 25% overall yield). Anal. (C₁₇H₁₇FK₃O₈PS.0.4KBr.2H₂O) C, H; ¹H NMR (400 MHz, D₂O): δ 1.50-1.90 (m, 4H, —CH₂CH₂—); 2.30-2.50 (m, 2H, PhCH₂), 2.60-2.70 (m, 1H, CHSO₃K), 3.62 (s, 3H, OMe), 6.65-7.00 (m, 7H, aromatic); ³¹P NMR (D₂O): δ 13.6.

Methods for making compounds disclosed herewith are known to a person of skill in the art and/or can be obtained through the references incorporated by reference.

### Computational Methods

CoMSIA analysis was performed with default settings in Sybyl (16) (version 7.3). All compounds were geometrically optimized, using the MMFF94x forcefield, then aligned in the program MOE (13), utilizing the flexible alignment module (14). The alignment was carried out by performing up to 1,000 flexible refinement iterations using a gradient test of 0.01 to 1.0 with hydrophobe, log P, and partial charge similarity features, as well as the default options (H-bond donor, acceptor, aromaticity, polar hydrogens and volume). The alignments were exported into the Sybyl program, where atomic charges were determined by using the Gasteiger-Marsili method (22). CoMSIA indices were calculated on a rectangular grid containing each of the sets of aligned molecules using steric, electrostatic, hydrophobic, H-donor and acceptor fields, using default grid spacing and probe atoms. We then used a partial least-squares (PLS) method to correlate the 3D structural features with activity. The optimal number of components was determined with the SAMPLS cross validation method 23. Each of the QSAR models was then validated by performing five training/test sets.

For cell activity predictions, a set of 117 molecular descriptors, including S log P (17), were computed within MOE and exported to MATLAB (24) for a combinatorial descriptor search. The entire descriptor space was searched exhaustively to find the combinations of descriptors, which gave the best regression coefficient (highest R²) for the equation:

pIC₅₀(pigment)=a·pIC₅₀(Enzyme)+b·B+c·C+d

where B and C are MOE descriptors and a-d are coefficients. A leave-two-out cross-validation was performed to test the predictivity of the model, where all combinations of 2 compounds were excluded from the data set and the descriptor combinations reevaluated for the remaining (training set) compounds. The regression equation obtained from each run was then used to calculate the cell activity of the left out compounds (the test set) and the R² from all such leave-two-out predictions is reported in the text. Finally, a scrambling analysis was performed in which the cell activity values for all the compounds were scrambled, and the leave-two-out cross-validation performed using the scrambled data set.

### CrtM Enzyme Inhibition Assay

The expression and purification of the S. aureus CrtM as well as the inhibition assays were carried out by using our previous methods (6). In brief, CrtM with a histidine tag was overexpressed in E. coli BL21 (DE3) cells. After an overnight growth, an initial 50 mL culture was transferred into 1 L LB medium supplemented with 100 μg/mL ampicillin. Induction was carried out with 1 mM IPTG for four hours, when the cell culture reached an OD of 0.6 at 600 nm. Protein was purified with a Ni-NTA column, using a 100 mL linear gradient of 0-0.5 M imidazole in 50 mM Tris-HCl buffer at pH 7.4. Enzyme inhibition assays were carried out, in duplicate, in 96 well plates, with a total of 200 μL reaction mixture in each well. The reaction was monitored by using a continuous spectrophotometric assay for phosphate releasing enzymes (25). The reaction buffer contained 50 mM Tris-HCl, 1 mM MgCl₂, 450 μM FPP, pH 7.4. The compounds investigated were pre-incubated with 2 μg CrtM for 30 minutes at 20° C. The IC₅₀ values were obtained by fitting the inhibition data to a normal dose response curve, using Graph Pad PRISM® version 4.0 software for windows (Graph Pad Software Inc., San Diego, Calif., www.graphpad.com). Kᵢ was calculated based on the IC₅₀ value and the reported Kₘ of CrtM (26).

### Staphyloxanthin Biosynthesis Inhibition Assay

The S. aureus strain used was the WT clinical isolate (Pig1) (4). S. aureus was cultured in THB (1 mL) in the presence of inhibitor compounds for 72 h, in duplicate. Cells were then centrifuged and washed twice with PBS. STX was extracted with MeOH and the O.D. was determined at 450 nm using a Perkin Elmer MBA 2000 (Norwalk, Conn.) spectrophotometer. The IC₅₀ values were obtained by fitting the O.D. data to a normal dose-response curve, using Graph Pad PRISM®.

### Human SQS Enzyme Expression, Purification and Inhibition Assay

A DNA sequence encoding a double truncated protein lacking residues 31 at the N-terminus and 46 at the C-terminus was amplified using the following primers: 5′ CATATGGACCAGGACTCGCTCAGCAGC (SEQ ID NO:1) and 3′ GGATCCTCAATTCTGCGTCCGGATGGT (SEQ ID NO:2). The corresponding amplified insert was initially cloned in the vector pGEMT® (Promega). Plasmid was digested with the endonucleases NdeI and BamHI, and the resulting fragment was cloned into the bacterial expression vector pET-28a to give pET28a-HsSQS which was used to transform E. coli BL21 (DE3) RP strain (Novagen) for overexpression. This cloning procedure resulted in the addition of a six-histidine tag to the N-terminus of double truncated HsSQS.

Bacteria expressing the constructs were cultured in Luria-Bertani medium supplemented with kanamycin (30 μg/ml) and chloramphenicol (34 μg/ml) at 37° C., until the cells reached an OD of 0.4 at 600 nm, and were then induced at 37° C. for 4 h by incubation with 1 mM isopropyl-1-thio-β-D-galactopyranoside. Cells were harvested by centrifugation (10 min, 4000 rpm) and resuspended in 10 ml of lysis/elution buffer (20 mM NaH₂PO₃, pH 7.4, 10 mM CHAPS, 2 mM MgCl₂, 10% glycerol, 10 mM—mercaptoethanol, 500 mM NaCl, 10 mM imidazole, and a protease inhibitor cocktail (Roche), disrupted by sonication, and centrifuged at 16,000 rpm for 30 min. The supernatant (40 ml) was then applied to a HiTrap Nickel-Chelating HP column (Amersham Biosciences). Enzyme purification was performed according to the manufacturer's instructions using a Pharmacia FPLC system. Unbound protein was washed off with 50 mM imidazole, then the His6-HsSQS was eluted with 1M imidazole. Purity was confirmed by SDS-PAGE electrophoresis. Fractions containing the pure enzyme were pooled and dialyzed against buffer A (25 mM sodium phosphate pH 7.4, 20 mM NaCl, 2 mM dithiothreitol, 1 mM EDTA, 10% glycerol, 10% methanol), concentrated, then stored at −80° C.

SQS activity was based on measuring the conversion of [3H]FPP to [3H]squalene. Final assay concentrations were 50 mM MOPS (pH 7.4), 20 mM MgCl2, 5 mM CHAPS, 1% Tween 80, 10 mM DTT, 0.025 mg/mL BSA, 0.25 mM NADPH, and 7.5 ng of purified recombinant human SQS. The reaction was started with the addition of substrate (3HFPP, 0.1 nmol, 2.22×106 dpm) and the final volume of the reaction was 200 μL. After incubation at 37° C. for 5 min, 40 μL of 10 M NaOH were added to stop the reaction, followed by 10 μL of a (100:1) mixture of 98% EtOH and squalene. The resulting mixtures were mixed vigorously by vortexing, then 10 μL aliquots were applied to 2.5×10 cm channels of a silica gel thin layer chromatogram, and newly formed squalene was separated from unreacted substrate by chromatography in toluene-EtOAc (9:1). The region of the squalene band was scraped and immersed in Hydrofluor liquid scintillation fluid, and assayed for radioactivity. IC₅₀ values were calculated from the hyperbolic plot of percent of inhibition versus inhibitor concentration, using GraphPad PRISM®.

### Example 4

### Chemical Structures of Certain Compounds

TABLE 9
Chemical Structures of Certain Compounds

| Compound Indentifier | Chemical Structure |
|---|---|
| BPH-759 | 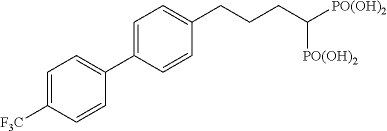 |
| BPH-674 | 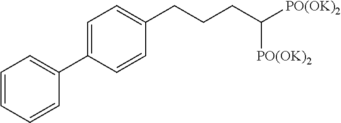 |

### REFERENCES

1. Bancroft, E. A. Antimicrobial resistance: It's not just for hospitals. JAMA 2007, 298, 1803-1804.
2. Klevens, R. M.; Morrison, M. A.; Nadle, J.; Petit, S.; Gershman, K.; Ray, S.; Harrison, L. H.; Lynfield, R.; Dumyati, G.; Townes, J. M.; Craig, A. S.; Zell, E. R.; Fosheim, G. E.; McDougal, L. K.; Carey, R. B.; Fridkin, S. K. Invasive methicillinresistant Staphylococcus aureus infections in the United States. JAMA 2007, 298, 1763-1771.
3. National Research Council, Treating Infectious Diseases in a Microbial World: Report of Two Workshops on Novel Antimicrobial Therapeutics. National Academies Press: Washington, D.C., 2006; p 21.
4. Liu, G. Y.; Essex, A.; Buchanan, J. T.; Datta, V.; Hoffman, H. M.; Bastian, J. F.; Fierer, J.; Nizet, V. Staphylococcus aureus golden pigment impairs neutrophil killing and promotes virulence through its antioxidant activity. J. Exp. Med. 2005, 202, 209-215.
5. Clauditz, A.; Resch, A.; Wieland, K. P.; Peschel, A.; Gotz, F. Staphyloxanthin plays a role in the fitness of Staphylococcus aureus and its ability to cope with oxidative stress. Infect. Immun. 2006, 74, 4950-4953.
6. Liu, C. I.; Liu, G. Y.; Song, Y.; Yin, F.; Hensler, M. E.; Jeng, W. Y.; Nizet, V.; Wang, A. H.; Oldfield, E. A cholesterol biosynthesis inhibitor blocks Staphylococcus aureus virulence. Science 2008, 319, 1391-1394.
7. Sharma, A.; Slugg, P. H.; Hammett, J. L.; Jusko, W. J. Clinical pharmacokinetics and pharmacodynamics of a new squalene synthase inhibitor, BMS-188494, in healthy volunteers. J. Clin. Pharmacol. 1998, 38, 1116-1121.
8. Sharma, A.; Slugg, P. H.; Hammett, J. L.; Jusko, W. J. Estimation of oral bioavailability of a long half-life drug in healthy subjects. Pharm. Res. 1998, 15, 1782-1786.
9. Ciosek, C. P., Jr.; Magnin, D. R.; Harrity, T. W.; Logan, J. V.; Dickson, J. K., Jr.; Gordon, E. M.; Hamilton, K. A.; Jolibois, K. G.; Kunselman, L. K.; Lawrence, R. M.; et al. Lipophilic 1,1-bisphosphonates are potent squalene synthase inhibitors and orally active cholesterol lowering agents in vivo. J. Biol. Chem. 1993, 268, 24832-24837.
10. Magnin, D. R.; Biller, S. A.; Chen, Y.; Dickson, J. K., Jr.; Fryszman, O. M.; Lawrence, R. M.; Logan, J. V.; Sieber-McMaster, E. S.; Sulsky, R. B.; Traeger, S.C.; Hsieh, D.C.; Lan, S. J.; Rinehart, J. K.; Harrity, T. W.; Jolibois, K. G.; Kunselman, L. K.; Rich, L. C.; Slusarchyk, D. A.; Ciosek, C. P., Jr. α-Phosphonosulfonic acids: potent and selective inhibitors of squalene synthase. J. Med. Chem. 1996, 39, 657-660.
11. Lawrence, R. M.; Biller, S. A.; Dickson, J. K.; Logan, J. V. H.; Magnin, D. R.; Sulsky, R. B.; DiMarco, J. D.; Gougoutas, J. Z.; Beyer, B. D.; Taylor, S. C.; Lan, S. J.; Ciosek, C. P.; Harrity, T. W.; Jolibois, K. G.; Kunselman, L. K.; Slusarchyk, D. A. Enantioselective synthesis of α-phosphono sulfonate squalene synthase inhibitors: Chiral recognition in the interactions of an α-phosphono sulfonate inhibitor with squalene synthase. J. Am. Chem. Soc. 1996, 118, 11668-11669.
12. Klebe, G.; Abraham, U.; Mietzner, T. Molecular similarity indices in a comparative analysis (CoMSIA) of drug molecules to correlate and predict their biological activity. J. Med. Chem. 1994, 37, 4130-4146.
13. Molecular Operating Environment (MOE), Chemical Computing Group, Inc.: Montreal, Quebec, 2006.
14. Labute, P.; Williams, C.; Feher, M.; Sourial, E.; Schmidt, J. M. Flexible alignment of small molecules. J. Med. Chem. 2001, 44, 1483-1490.
15. Mao, J.; Mukherjee, S.; Zhang, Y.; Cao, R.; Sanders, J. M.; Song, Y.; Zhang, Y.; Meints, G. A.; Gao, Y. G.; Mukkamala, D.; Hudock, M. P.; Oldfield, E. Solid-state NMR, crystallographic, and computational investigation of bisphosphonates and farnesyl diphosphate synthase-bisphosphonate complexes. J. Am. Chem. Soc. 2006, 128, 14485-14497.
16. Sybyl, Tripos: St. Louis, Mo., 2007.
17. Wildman, S. A.; Crippen, G. M. Prediction of physicochemical parameters by atomic contributions. J. Chem. Inf. Comp. Sci. 1999, 39, 868-873.
18. Ma, D.; Cai, Q. N,N-dimethyl glycine-promoted Ullmann coupling reaction of phenols and aryl halides. Org. Lett. 2003, 5, 3799-3802.
19. Teulade, M.-P.; Savignac, P.; Aboujaoude, E. E.; Collignon, N. Carbanions phosphonates [alpha]-lithiium: synthèse, basicité comparée et stabilité à l'autocondensation. J. Organomet. Chem. 1986, 312, 283-295.
20. Yu, Y.; Srogl, J.; Liebeskind, L. S. Cu(I)-mediated reductive amination of boronic acids with nitroso aromatics. Org. Lett. 2004, 6, 2631-2634.
21. Sanders, J. M.; Song, Y.; Chan, J. M.; Zhang, Y.; Jennings, S.; Kosztowski, T.; Odeh, S.; Flessner, R.; Schwerdtfeger, C.; Kotsikorou, E.; Meints, G. A.; Gomez, A. O.; Gonzalez-Pacanowska, D.; Raker, A. M.; Wang, H.; van Beek, E. R.; Papapoulos, S. E.; Morita, C. T.; Oldfield, E. Pyridinium-1-yl bisphosphonates are potent inhibitors of farnesyl diphosphate synthase and bone resorption. J. Med. Chem. 2005, 48, 2957-2963.
22. Gasteiger, J.; Marsili, M. Iterative partial equalization of orbital electronegativity—a rapid access to atomic charges. Tetrahedron 1980, 36, 3219-3228.
23. Bush, B. L.; Nachbar, R. B., Jr. Sample-distance partial least squares: PLS optimized for many variables, with application to CoMFA. J. Comput. Aided Mol. Des. 1993, 7, 587-619.
24. MATLAB Version 7.4, The Mathworks, Inc.: Natick, Mass., 2007.
25. Rieger, C. E.; Lee, J.; Turnbull, J. L. A continuous spectrophotometric assay for aspartate transcarbamylase and ATPases. Anal. Biochem. 1997, 246, 86-95.
26. Ku, B.; Jeong, J. C.; Mijts, B. N.; Schmidt-Dannert, C.; Dordick, J. S. Preparation, characterization, and optimization of an in vitro C30 carotenoid pathway. Appl. Environ. Microbiol. 2005, 71, 6578-6583.

Any sequence listing information is part of the specification and disclosure herewith.

### STATEMENTS REGARDING INCORPORATION BY REFERENCES AND VARIATIONS

The reference, Liu C I, Liu G Y, Song Y, Yin F, Hensler M E, Jeng W Y, Nizet V, Wang A H, Oldfield E, 2008, A Cholesterol Biosynthesis Inhibitor Blocks Staphylococcus Aureus Virulence, Science, volume 319, pages 1391-1394, is incorporated herein by reference in its entirety.

U.S. Provisional Application Ser. No. 60/672,359 filed Apr. 18, 2005 entitled Novel Antimicrobial Therapy for Staphylococcus Aureus Infections by Nizet et al. and related International Application number PCT/US2006/014486 entitled Antimicrobial Therapy for Bacterial Infections filed Apr. 17, 2006 and published as International Application Publication number WO/2007/075186 on May 7, 2007. U.S. Provisional Application Ser. No. 60/800,654 filed May 12, 2006 entitled Antimicrobial Therapy for Bacterial Infections by Nizet et al. and related International Application number PCT/US2007/011466 filed May 11, 2007 entitled Antimicrobial Therapy for Bacterial Infections published as International Application Publication number WO/2007/133712 on Nov. 22, 2007. U.S. patent application Ser. No. 11/918,584 filed Oct. 15, 2007 by Nizet et al. for Antimicrobial therapy for bacterial infections; published as US Patent Application Pub. No. 20090042976 on Feb. 12, 2009. U.S. patent application Ser. No. 12/299,934 filed Feb. 10, 2009 by Nizet et al. for Antimicrobial therapy for bacterial infections; published as US Patent Application Pub. No. 20090306021 on Dec. 10, 2009.

EP0710665A1 to Pendri et al., Bristol-Myers Squibb, 8 May 1996; U.S. Pat. No. 5,965,553 to Bell, et al. Oct. 12, 1999.

When a group of chemical species is disclosed herein, it is understood that all individual members of that group and all subgroups, including any structural isomers, enantiomers, and diastereomers of the group members, are disclosed separately. When a Markush group or other grouping is used herein, all individual members of the group and all combinations and subcombinations possible of the group are intended to be individually included in the disclosure.

Compounds described herein may exist in one or more isomeric forms, e.g., structural or optical isomers. When a compound is described herein such that a particular isomer, enantiomer or diastereomer of the compound is not specified, for example, in a formula or in a chemical name, that description is intended to include each isomers and enantiomer (e.g., cis/trans isomers, R/S enantiomers) of the compound described individual or in any combination.

The compounds of this invention may contain one or more chiral centers. Accordingly, this invention is intended to include racemic mixtures and non-racemic mixtures enriched in one or more steroisomer. The invention is intended to include individual enantiomers and diastereomers substantially free (less than 95% and preferably less than 99% by weight) of other enantiomers and/or diastereomers.

Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Isotopic variants, including those carrying radioisotopes, may also be useful in diagnostic assays and in therapeutics. Methods for making such isotopic variants are known in the art.

Specific names of compounds are intended to be exemplary, as it is known that one of ordinary skill in the art can name the same compounds differently.

Molecules disclosed herein may contain one or more ionizable groups [groups from which a proton can be removed (e.g., —COON) or added (e.g., amines) or which can be quaternized (e.g., amines)]. All possible ionic forms of such molecules and salts thereof are intended to be included individually in the disclosure herein. Additionally certain compounds of the invention may be cationic or anionic, e.g., contain cationic sulfonium or phosphonium groups. It is understood that such compounds can be in the form of salts with appropriate counterions. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt. Exemplary anions for such salts include halides (e.g., Cl⁻, Br⁻), carboxylates (e.g., R—CO₂⁻, where R is optionally substituted alkyl or aryl).

Exemplary cations for such salts include alkali metal cations (e.g., Na⁺, K⁺, etc.), alkaline earth cations (e.g., Mg²⁺, Ca²⁺, etc.), ammonium cations N(R)₄⁺, where each R is H, optionally substituted alkyl or aryl (e.g., NH₄⁺, N(CH₃)₄⁺.

Every formulation or combination of components described or exemplified herein can be used to practice the invention, unless otherwise stated.

Whenever a range is given in the specification, for example, a temperature range, pressure range, a time range, a range of values for a given variable, or a composition or concentration range, all intermediate ranges and subranges, as well as all individual values included in the ranges given are intended to be included in the disclosure. Unless otherwise noted all ranges noted herein are inclusive of the lower and upper range value listed. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, for example if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. The broad term comprising is intended to encompass the narrower consisting essentially of and the even narrower consisting of: Thus, in any recitation herein of a phrase “comprising one or more claim element” (e.g., “comprising A and B), the phrase is intended to encompass the narrower, for example, “consisting essentially of A and B” and “consisting of A and B.” Thus, the broader word “comprising” is intended to provide specific support in each use herein for either “consisting essentially of” or “consisting of:” The invention illustratively described herein suitably may be practiced in the absence of any element or elements, limitation or limitations which is not specifically disclosed herein.

As used herein, “administering a therapeutically effective amount” is intended to include methods of giving or applying a pharmaceutical composition of the disclosure to a subject that allow the composition to perform its intended therapeutic function. The therapeutically effective amounts will vary according to factors, such as the degree of infection in a subject, the age, sex, and weight of the individual. Dosage regima can be adjusted to provide the optimum therapeutic response. For example, several divided doses can be administered daily or the dose can be proportionally reduced as indicated by the exigencies of the therapeutic situation.

A therapeutically effective amount can be measured as the amount sufficient to decrease a subject's symptoms (e.g., dermatitis or rash by measuring the frequency of severity of skin sores). Typically, the subject is treated with an amount of a therapeutic composition of the invention sufficient to reduce a symptom of a disease or disorder by at least 50%, 90% or 100%. Generally, the optimal dosage will depend upon the disorder and factors such as the weight of the subject, the type of bacteria, virus or fungal infection, the weight, sex, and degree of symptoms. Nonetheless, suitable dosages can readily be determined by one skilled in the art. Typically, a suitable dosage is 0.5 to 40 mg/kg body weight, e.g., 1 to 8 mg/kg body weight. As mentioned previously, the compositions and methods of the invention can include the use of additional (e.g., in addition to a carotenoid biosynthesis inhibitor) therapeutic agents (e.g., an inhibitor of TNF, an antibiotic, and the like). The carotenoid biosynthesis inhibitor, other therapeutic agent (s), and/or antibiotic (s) can be administered, simultaneously, but may also be administered sequentially. Suitable antibiotics include aminoglycosides (e.g., gentamicin) beta-lactams (e.g., penicillins and cephalosporins), quinolones (e.g., ciprofloxacin), and novobiocin. Generally, the antibiotic is administered in a bactericidal, antiviral and/or antifungal amount. Their effects can also be augmented by co-administration with an inhibitor of flavohemoglobin, (Helmick et al., Imidazole antibiotics inhibit the nitric oxide dioxygenase function of microbial flavohemoglobin. Antimicrob Agents Chemother, 2005, 49 (5): 1837-43, and Sud et al., Action of antifungal imidazoles on Staphylococcus aureus, Antimicrob Agents Chemother, 1982, 22 (3): 470-4), increasing the efficacy of NO— based S. aureus killing by macrophages, and optionally triple combination therapies comprising one squalene synthase inhibitor, one flavohemoglobin (nitric oxide dioxygenase) inhibitor such as an azole (miconazole, econazole, clortrimazole, and ketoconazole) and one antibiotic as described above, may be applied to a patient in need of therapy.

One of ordinary skill in the art will appreciate that starting materials, reagents, synthetic methods, purification methods, analytical methods, assay methods, substrates, and solids other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by examples, preferred embodiments and optional features, modification and variation of the concepts herein disclosed may be resorted to by those skilled in the art, and that such modifications and variations are considered to be within the scope of this invention as defined by the appended claims.

All references cited herein are hereby incorporated by reference herein in their entirety. In case of any discrepancy between disclosure in a reference cited and that of this specification, the present disclosure and specification takes precedence. Some references provided herein are incorporated by reference to provide details concerning sources of starting material, methods of synthesis, methods of purification, methods of analysis; as well as additional uses of the invention.

The invention has been described with reference to various specific and preferred embodiments and techniques. However, it should be understood that many variations and modifications may be made while remaining within the spirit and scope of the invention. It will be apparent to one of ordinary skill in the art that compositions, methods, devices, device elements, materials, procedures and techniques other than those specifically described herein can be applied to the practice of the invention as broadly disclosed herein without resort to undue experimentation. All art-known functional equivalents of compositions, methods, devices, device elements, materials, procedures and techniques described herein are intended to be encompassed by this invention. Whenever a range is disclosed, all subranges and individual values are intended to be encompassed as if separately set forth. This invention is not to be limited by the embodiments disclosed, including any shown in the drawings or exemplified in the specification, which are given by way of example or illustration and not of limitation. The scope of the invention shall be limited only by the claims.

1. A compound of the formula 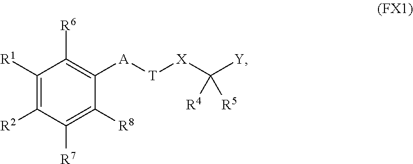
wherein:
Y is selected from the group consisting of: —P(O)(O⁻M¹)(O⁻M²), —P(O)(OH)₂, —P(O)(OH)(O⁻M³), —SO₃H, —SO₃⁻M⁴, —C(O)O⁻M⁵ and —COOH;
X is selected from the group consisting of: —C(O)—, —S(O₂)—, —P(O)(O⁻M⁶)-, and —CH₂—;
T is selected from the group consisting of: —O—, —CH₂—, and —NR³—;
A is a bridging diradical selected from the group consisting of: —(CH₂)ₙ—, —(C₆H₄)ₙ—, —(CF₂)ₙ—, —(CCl₂)ₙ—, —(CBr₂)ₙ—, alkylene, substituted alkylene, arylene, substituted arylene, alkylenearylene, substituted alkylenearylene, arylenealkylene, substituted arylenealkylene, alkylenearylenealkylene, substituted alkylenearylenealkylene, oxyalkylene, substituted oxyalkylene, oxyalkylenearylene, substituted oxyalkylenearylene, oxyarylene, substituted oxyarylene, oxyarylenealkylene, and substituted oxyarylenealkylene;
n is 0, 1, 2, 3, 4, 5, 6, 7, 8, 9, or 10;
M¹, M², M³, M⁴, M⁵, M⁶, M⁷ are each independently a pharmaceutically acceptable cation;
R³ is selected from the group consisting of: —H, —OH, —O⁻M⁷, aryl, substituted aryl, alkyl, substituted alkyl, —COOH, —COO⁻, —CO—NH₂, —(CH₂)ₙ—O—CO—, and halo;
R⁴ and R⁵ are each independently selected from the group consisting of —H, —OH, —O⁻M⁷, aryl, substituted aryl, alkyl, substituted alkyl, —COOH, —COO⁻, —CO—NH₂, —(CH₂)ₙ—COOH, —(CH₂)ₙ—COO⁻, and halo;
R¹, R², R⁶, R⁷, R⁸ are each independently selected from the group consisting of: aryl, substituted aryl, alkyl, substituted alkyl, —COOH, —COO⁻, —CO—NH₂, —(CH₂)ₙ—O—CO—, halo, and the following substituents:

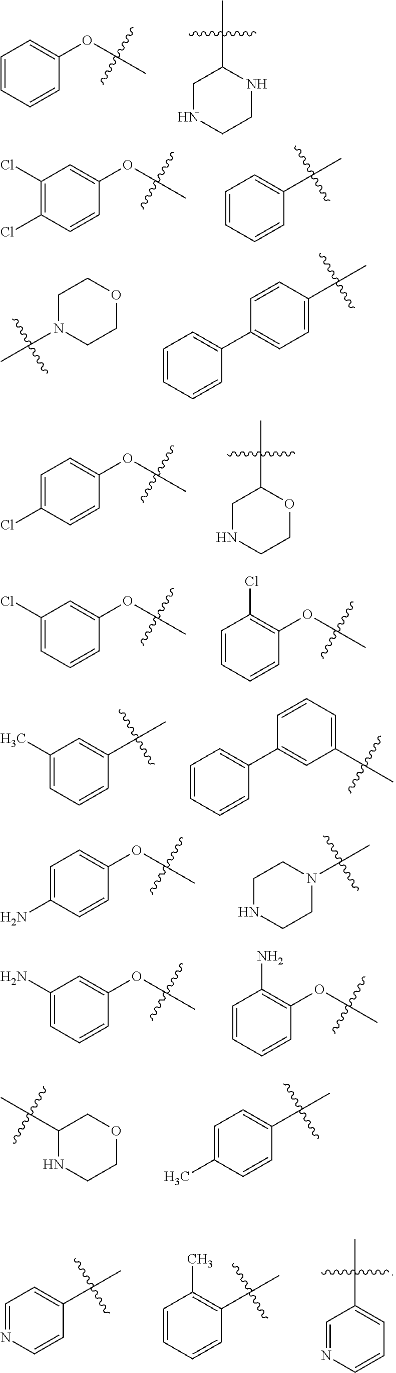

2. The compound of claim 1 having the formula selected from the group consisting of:

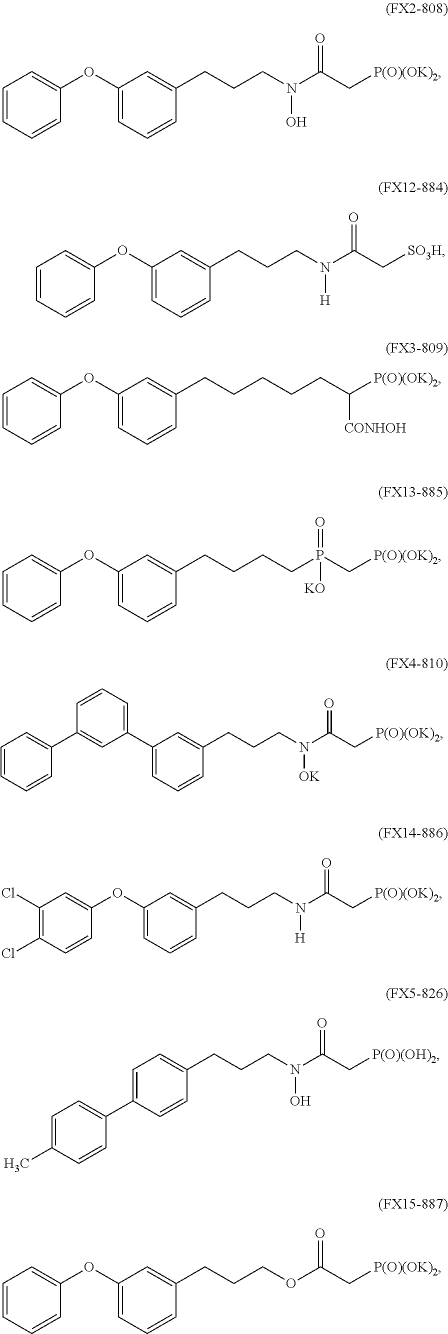

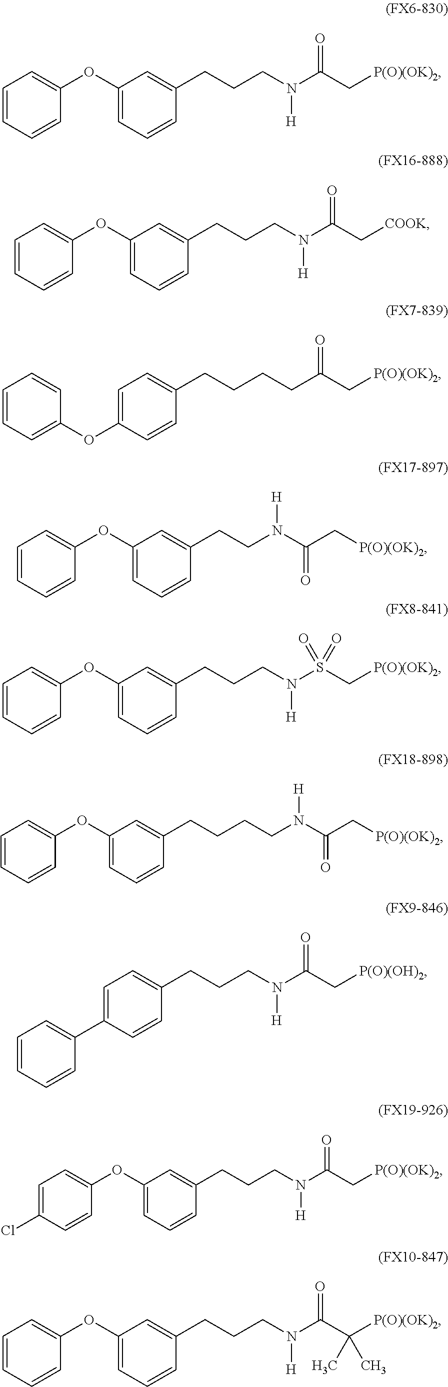

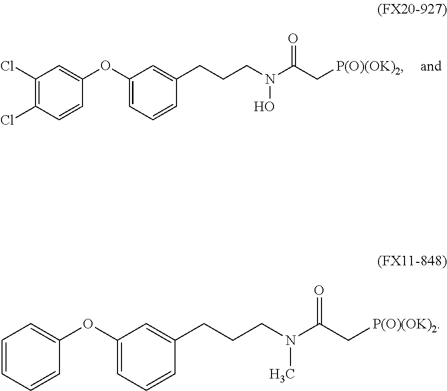

3. A method of inhibiting an infection comprising contacting an infectious agent with the compound of claim 1.

4. The method of claim 3 wherein the infectious agent is contacted with the compound in combination with at least one antibiotic.

5. The method of claim 4, wherein the antibiotic is or belongs to a class selected from the group consisting of aminoglycosides, penicillins, cephalosporins, carbapenems, monobactams, quinolones, tetracyclines, glycopeptides, chloramphenicol, clindamycin, trimethoprim, sulfamethoxazole, nitrofurantoin, rifampin and mupirocin.

6. The method of claim 4, wherein the antibiotic is selected from the group consisting of amikacin, gentamicin, kanamycin, netilmicin, tobramycin, streptomycin, azithromycin, clarithromycin, erythromycin, erythromycin estolate, erythromycin ethylsuccinate, erythromycin gluceptatellactobionate, erythromycin stearate, penicillin G, penicillin V, methicillin, nafcillin, oxacillin, cloxacillin, dicloxacillin, ampicillin, amoxicillin, ticarcillin, carbenicillin, mezlocillin, azlocillin, piperacillin, cephalothin, cefazolin, cefaclor, cefamandole, cefoxitin, cefuroxime, cefonicid, cefmetazole, cefotetan, cefprozil, loracarbef, cefetamet, cefoperazone, cefotaxime, ceftizoxime, ceftriaxone, ceftazidime, cefepime, cefixime, cefpodoxime, cefsulodin, imipenem, aztreonam, fleroxacin, nalidixic acid, norfloxacin, ciprofloxacin, ofloxacin, enoxacin, lomefloxacin, cinoxacin, doxycycline, minocycline, tetracycline, vancomycin, and teicoplanin.

7. The method of claim 3 wherein the infection is a microbial infection.

8. The method of claim 3 wherein the infectious agent is a Staphylococcus species.

9. The method of claim 3 wherein the infectious agent is Staphylococcus aureus.

10. (canceled)

11. A method of inhibiting growth of a microbe comprising contacting the microbe with the compound of claim 1.

12. The method of claim 11 wherein the microbe is contacted with the compound in combination with at least one antibiotic.

13-14. (canceled)

15. The method of claim 11 wherein the microbe is a Staphylococcus species.

16. A method of selectively inhibiting microbial activity comprising contacting a microbe with a compound wherein the compound is capable of inhibiting a CrtM activity or STX biosynthesis and has a limited capability for inhibiting or substantially inhibiting human cholesterol biosynthesis or human squalene synthase (hSQS).

17-19. (canceled)

20. The method of claim 16 wherein the compound is capable of inhibiting STX biosynthesis with an IC₅₀ level of less than or equal to 50 μM or is capable of inhibiting CrtM activity with an IC₅₀ level of less than or equal to 500 μM.

21. The method of claim 16 wherein the IC₅₀ level for STX is less than or equal to 10 μM.

22. The method of claim 16 wherein the IC₅₀ level for STX is less than or equal to 1 μM.

23. (canceled)

24. (canceled)

25. The method of claim 16 wherein the IC₅₀ level for CrtM is less than or equal to 100 μM.

26. The method of claim 16 wherein the limited capability for inhibiting or substantially inhibiting human cholesterol biosynthesis or human squalene synthase is capable of being reflected by a relative selectivity of the compound for inhibiting CrtM activity or inhibiting STX biosynthesis in comparison to inhibiting human squalene synthase (hSQS), wherein the compound is capable of demonstrating said relative selectivity in the form of a selectivity ratio of [IC₅₀(hSQS)/IC₅₀(CrtM)] for the compound with respect to that of a reference compound BPH-652 (FX24B-04-652), and wherein said relative selectivity value is greater than 1; or wherein said limited capability is reflected by the compound being capable of demonstrating an absolute ratio of [IC₅₀(hSQS)/IC₅₀(CrtM)] wherein such absolute ratio is greater than 0.005.

27-31. (canceled)

32. The method of claim 16
wherein the compound is of the formula FX21-I or FX22-II: 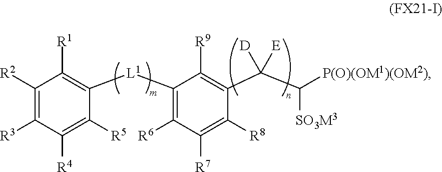
wherein:
m is 0, 1, 2 or 3;
n is 1, 2, 3, 4, 5, 6, 7, 8, 9, or 10;
D and E are each independently selected from the group consisting of: H, aryl, substituted aryl, alkyl, substituted alkyl, carboxyl, aminocarbonyl, alkylsulfonylaminocarboxyl, alkoxycarbonyl, and halo;
M¹, M², and M³ are each independently a pharmaceutically acceptable cation;
R¹ is selected from the group consisting of: H, aryl, substituted aryl, alkyl, substituted alkyl, carboxyl, aminocarbonyl, alkylsulfonylaminocarboxyl, alkoxycarbonyl, and halo, or R¹ and R², together with the carbons to which they are bound, can be joined to form a 4 to 7 membered ring or a substituted 4 to 7 membered ring;
R² is selected from the group consisting of: H, aryl, substituted aryl, alkyl, substituted alkyl, carboxyl, aminocarbonyl, alkylsulfonylaminocarboxyl, alkoxycarbonyl, and halo, or R² and R¹, together with the carbons to which they are bound, can be joined to form a 4 to 7 membered ring or a substituted 4 to 7 membered ring, or R² and R³, together with the carbons to which they are bound, can be joined to form a 4 to 7 membered ring or a substituted 4 to 7 membered ring;
R³ is selected from the group consisting of: H, aryl, substituted aryl, alkyl, substituted alkyl, carboxyl, aminocarbonyl, alkylsulfonylaminocarboxyl, alkoxycarbonyl, and halo, or R³ and R², together with the carbons to which they are bound, can be joined to form a 4 to 7 membered ring or a substituted 4 to 7 membered ring, or R³ and R⁴, together with the carbons to which they are bound, can be joined to form a 4 to 7 membered ring or a substituted 4 to 7 membered ring;
R⁴ is selected from the group consisting of: H, aryl, substituted aryl, alkyl, substituted alkyl, carboxyl, aminocarbonyl, alkylsulfonylaminocarboxyl, alkoxycarbonyl, and halo, or R⁴ and R³, together with the carbons to which they are bound, can be joined to form a 4 to 7 membered ring or a substituted 4 to 7 membered ring;
R⁵, R⁶, R⁷, R⁸, and R⁹ are each independently selected from the group consisting of: H, aryl, substituted aryl, alkyl, substituted alkyl, carboxyl, aminocarbonyl, alkylsulfonylaminocarboxyl, alkoxycarbonyl, and halo;
L¹ is —S—, —SO—, —SO₂—, —O—, —N(R¹⁹)—, or —C(R²⁰)(R²¹)—; wherein R¹⁹, R²⁰ and R²¹ are each independently selected from the group consisting of: H, aryl, substituted aryl, alkyl, substituted alkyl, carboxyl, aminocarbonyl, alkylsulfonylaminocarboxyl, alkoxycarbonyl, and halo; 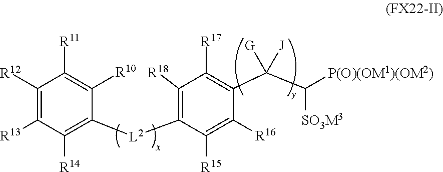
wherein:
x is 0, 1, 2, or 3;
y is 1, 2, 3, 4, 5, 6, 7, 8, 9, or 10;
G and J are independently selected from the group consisting of: H, aryl, substituted aryl, alkyl, substituted alkyl, carboxyl, aminocarbonyl, alkylsulfonylaminocarboxyl, alkoxycarbonyl, and halo;
M¹, M², and M³ are each independently a pharmaceutically acceptable cation;
R¹⁰ is selected from the group consisting of: H, aryl, substituted aryl, alkyl, substituted alkyl, carboxyl, aminocarbonyl, alkylsulfonylaminocarboxyl, alkoxycarbonyl, and halo, or R¹⁰ and R¹¹, together with the carbons to which they are bound, can be joined to form a 4 to 7 membered ring or a substituted 4 to 7 membered ring;
R¹¹ is selected from the group consisting of: H, aryl, substituted aryl, alkyl, substituted alkyl, carboxyl, aminocarbonyl, alkylsulfonylaminocarboxyl, alkoxycarbonyl, and halo, or R¹¹ and R¹⁰, together with the carbons to which they are bound, can be joined to form a 4 to 7 membered ring or a substituted 4 to 7 membered ring, or R¹¹ and R¹², together with the carbons to which they are bound, can be joined to form a 4 to 7 membered ring or a substituted 4 to 7 membered ring;
R¹² is selected from the group consisting of: H, aryl, substituted aryl, alkyl, substituted alkyl, carboxyl, aminocarbonyl, alkylsulfonylaminocarboxyl, alkoxycarbonyl, and halo, or R¹² and R¹¹, together with the carbons to which they are bound, can be joined to form a 4 to 7 membered ring or a substituted 4 to 7 membered ring, or R¹² and R¹³, together with the carbons to which they are bound, can be joined to form a 4 to 7 membered ring or a substituted 4 to 7 membered ring;
R¹³ is selected from the group consisting of: H, aryl, substituted aryl, alkyl, substituted alkyl, carboxyl, aminocarbonyl, alkylsulfonylaminocarboxyl, alkoxycarbonyl, and halo, or R¹³ and R¹², together with the carbons to which they are bound, can be joined to form a 4 to 7 membered ring or a substituted 4 to 7 membered ring;
R¹⁴, R¹⁵, R¹⁶, R¹⁷, and R¹⁸ are each independently selected from the group consisting of: H, aryl, substituted aryl, alkyl, substituted alkyl, carboxyl, aminocarbonyl, alkylsulfonylaminocarboxyl, alkoxycarbonyl, and halo;
L² is —S—, —SO—, —SO₂—, —O—, —N(R²²)—, or —C(R²³)(R²⁴)—; wherein R²², R²³ and R²⁴ are each independently selected from the group consisting of: H, aryl, substituted aryl, alkyl, substituted alkyl, carboxyl, aminocarbonyl, alkylsulfonylaminocarboxyl, alkoxycarbonyl, and halo.

33. The method of claim 32 wherein the compound is not a compound of the formula selected from the group consisting of:

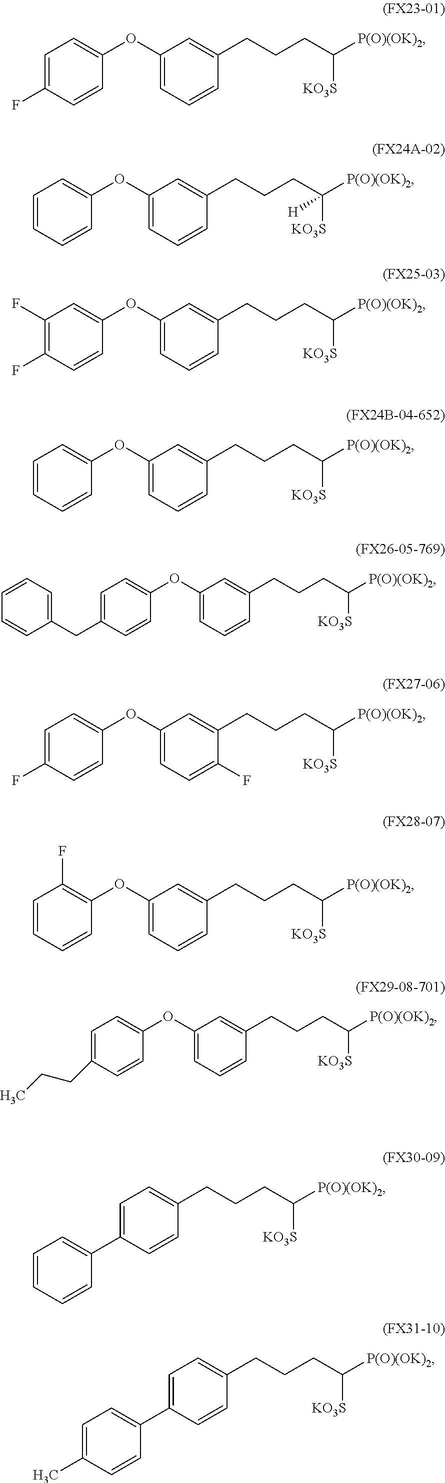

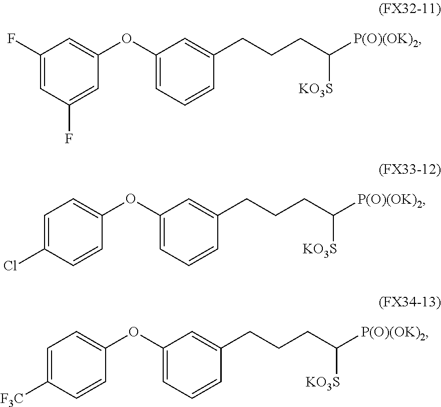

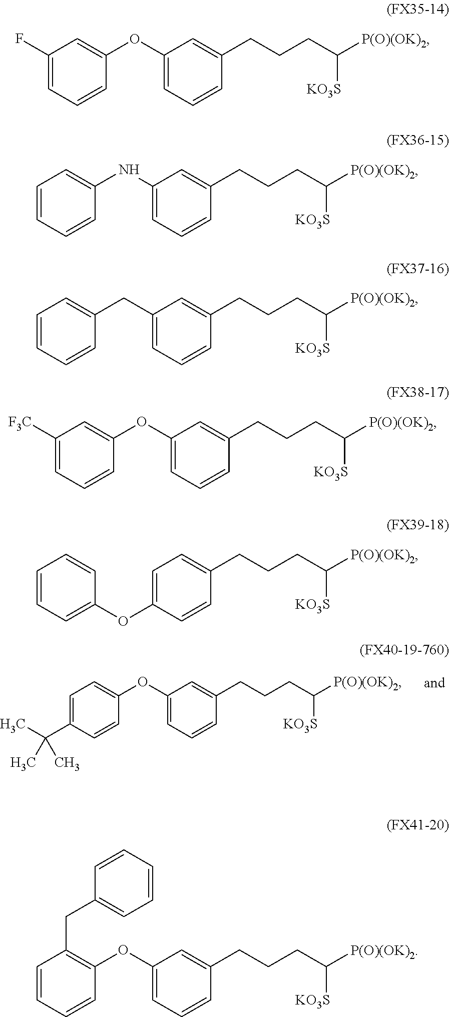

34-38. (canceled)
